SEQUENCE LISTING (212 sequences)

SEQ ID NO: 1 - protein, 158 aa, human
HHLLASDEEIQDVSGTWYLKAMTVDREFPEMNLESVTPMTLTTLEGGNLEAKVTMLISGRCQEVKAVLEKTDEPGKYTADGGKHVAYIIRSHVKDHYIFYCEGELHGKPVRGVKLVGRDPKNNLEALEDFEKAAGARGLSTESILIPRQSETCSPGSD

SEQ ID NO: 2 - protein, 178 aa, human
QDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 3 - protein, 178 aa, artificial sequence
  Lipocalin mutein
QDSTSDLIPAPPLSKVPLQQNFQDNQFQGKWYVVGLAGNAILREDKDPQKMYATIYELKEDKSYDVTSVLFRKKKCDYWIETFVPGSQPGEFTLGNIKSYPGLTSYLVRVVSTNYNQHAMVFFKEVSQNREYFEITLYGRTKELTSELKENFIRFSKSLGLPENHIVFPVPIDQCIDG

SEQ ID NO: 4 - protein, 154 aa, artificial sequence
  Lipocalin mutein
ASDEEIQDVSGTWYLKAMTVDRECPEMNLESVTPMTLTTLEGGNLEAKVTMLISGRSQEVKAVLEKTDEPGKYTADGGKHVAYIIRSHVKDHYIFYSEGECHGKPVPGVWLVGRDPKNNLEALEDFEKAAGARGLSTESILIPRQSETSSPGSD

SEQ ID NO: 5 - protein, 4 aa, human
HHLL

SEQ ID NO: 6 - protein, 178 aa, artificial sequence
  Lipocalin mutein
QDSTSDLIPAPPLSKVPLQQNFQDNQFHGKWYVVGEAGNWGLREDKDPKKMAATIYELKEDKSYNVTDVQFIEKKCDYWIGTFVPGSQPGEFTLGAIKSEPGQTSNLVRVVSTNYNQHAMVFFKEVWQNRELFWITLYGRTKELTSELKENFIRFSKSLGLPENHIVFPVPIDQCIDG

SEQ ID NO: 7 - protein, 178 aa, artificial sequence
  Lipocalin mutein
QDSTSDLIPAPPLSKVPLQQNFQDNQFHGKWYVVGFAGNMWLREDKDPFKMGATIYELKEDKSYNVTWVWFEAKKCDYGINTFVPGSQPGEFTLGGIKSPPGMTSHLVRVVSTNYNQHAMVFFKEVFQNREWFWITLYGRTKELTSELKENFIRFSKSLGLPENHIVFPVPIDQCIDG

SEQ ID NO: 8 - protein, 178 aa, artificial sequence
  Lipocalin mutein
QDSTSDLIPAPPLSKVPLQQNFQDNQFHGKWYVVGWAGNTLLREDKDPTKMQATIYELKEDKSYNVTWVYFSEKKCNYHIETFVPGSQPGEFTLGTIKSYPGETSILVRVVSTNYNQHAMVFFKGVSQNREIFEITLYGRTKELTSELKENFIRFSKSLGLPENHIVFPVPIDQCIDG

SEQ ID NO: 9 - protein, 178 aa, artificial sequence
  Lipocalin mutein
QDSTSDLIPAPPLSKVPLQQNFQDNQFHGKWYVVGRAGNAELREDKDPEKMEATIYELKEDKSYNVTDVGFWQKKCIYVIHTFVPGSQPGEFTLGNIKSYPGTTSALVRVVSTNYNQHAMVFFKVVRQNREWFEITLYGRTKELTSELKENFIRFSKSLGLPENHIVFPVPIDQCIDG

SEQ ID NO: 10 - protein, 178 aa, artificial sequence
  Lipocalin mutein
QDSTSDLIPAPPLSKVPLQQNFQDNQFHGKWYVVGIAGNWWLREDKDPLKMYATIYELKEDKSYNVTHVMFMTKKCDYTIRTFVPGSQPGEFTLGNIKSIPGMTSLLVRVVSTNYNQHAMVFFKFVWQNREWFHITLYGRTKELTSELKENFIRFSKSLGLPENHIVFPVPIDQCIDG

SEQ ID NO: 11 - protein, 178 aa, artificial sequence
  Lipocalin mutein
QDSTSDLIPAPPLSKVPLQQNFQDNQFHGKWYVVGWAGNTLLREDKDPIKMQATIYELKEDKSYNVTWVYFSEKRCNYHIETSVPGSQPGEFTLGTIKSYPGETSILVRVVSTNYNQHAMVFFKGVSQNREIFEITLYGRTKELTSELKENFIRFSKSLGLPENHIVFPVPIDQCIDG

SEQ ID NO: 12 - protein, 178 aa, artificial sequence
  Lipocalin mutein
QDSTSDLIPAPPLSKVPLQQNFQDNQFHGKWYVVGWAGNTLRREDKNPTKMQATIYELKEDKSYNVTWVYFSEKKCNYHIETSVPGSQPGEFTLGTIKSYPGETSILVRVVSTNYNQHAMVFFKGVSQNREIFEITLYGRTKELTSELKENFIRFSKSLGLPENHIVFPVPIDQCIDG

SEQ ID NO: 13 - protein, 178 aa, artificial sequence
  Lipocalin mutein
QDSTSDLIPAPPLSKVPLQQNFQDNQFHGKWYVVGWAGNTLLREDKDPIKMQATIYELKEDKSYDVTWVYLSEKKCNYHIETSVPGSQPGEFTLGTIKSYPGETSILVRVVSTNYNQHAMVFFKGMSQNREIFEITLYGRTKELASELKENFIRFSKSLGLPENHIVFPVPIDQCIDG

SEQ ID NO: 14 - protein, 178 aa, artificial sequence
  Lipocalin mutein
QDSTSDLIPAPPLSKVPLQQNFQDNQFHGKWYVVGWAGNTLLREDKNPTKMQATIYELKEDKSYNATWVYLSEKKCNYHIETSVPGSQPGEFTLGTTKSYPGETPILVRVVSTNYNQHAMVFFKGVSQNREIFEITLYGRTKELTSELKENFIRFSKSLGLPENHIVFPVPIDQCIDG

SEQ ID NO: 15 - protein, 178 aa, artificial sequence
  Lipocalin mutein
QDSTSDLIPAPPLSKVPLQQNFQDNQFHGKWYVVGWAGNTLLREDKDPIKMQAIIYELKEDRSYNVTWVYFSEKKCNYHIETFVPGSQPGEFTLGTIKSYPGETPILVRVVSTNYNQHAMVFFKGVNQNREIFEIILYGRTKELTNELKENFIRFSKSLGLPENHIVFPVPIDQCIDG

SEQ ID NO: 16 - protein, 178 aa, artificial sequence
  Lipocalin mutein
QDSTSDLIPAPPLSKVPLQQNFQDNQFHGKWYVVGWAGNTLLREDKDPPKMQATIYELKEDRSYNVTWVYFSEKKCNYHIETSVPGSQPGEFTLGTIQSHPGETPILVRVVSTNYNQHAMVFFKGMGQNREIFEITLYGRTKELTSELKENFIRFSKSLGLPENHIVFPVPIDQCIDG

SEQ ID NO: 17 - protein, 178 aa, artificial sequence
  Lipocalin mutein
QDSTSDLIPAPPLSKVPLQQNFQDNQFHGKWYVVGRAGNEELREDKDPQKMEATIYELKEDKSYNVTDVGFSEKKCIYVIHTFVPGSQPGEFTLGNIKSYPGVTSALVRVVSTNYNQHAMVFFKVVKQNRELFEITLYGRTKELTSELKENFIRFSKSLGLPENHIVFPVPIDQCIDG

SEQ ID NO: 18 - protein, 178 aa, artificial sequence
  Lipocalin mutein
QDSTSDLIPAPPLSKVPLQQNFQDNQFHGKWYVVGRAGNDALREDKDPEKMEATIYELKEDKSYNVTDVGFVQKKCMYVIHTFVPGSQPGEFTLGNIKSYPGVTSALVRVVSTNYNQHAMVFFKLVKQNRELFEITLYGRTKELTSELKENFIRFSKSLGLPENHIVFPVPIDQCIDG

SEQ ID NO: 19 - protein, 178 aa, artificial sequence
  Lipocalin mutein
QDSTSDLIPAPPLSKVPLQQNFQDNQFHGKWYVVGRAANDRLREDKDPEKMEATIYELKEDKSYNVTDVGFSRKKCIYVIHTFVPGSQPGEFTLGNIKSYPGTTSGLVRVVSTNYNQHAMVFFKVVGQNRESFEITLYGRTKELTSELKENFIRFSKSLGLPENHIVFPVPIDQCIDG

SEQ ID NO: 20 - protein, 178 aa, artificial sequence
  Lipocalin mutein
QDSTSDLIPAPPLSKVPLQQNFQDNQFHGKWYVVGRAGNDELREDKDPEKMEATIYELKEDKSYNVTDVGFVQKKCMYVIHTFVPGSQPGEFTLGNIKSYPGVTSGLVRVVSTNYNQHAMVFFKVVRQNRELFEITLYGRTKELTSELKENFIRFSKSLGLPENHIVFPVPIDQCIDG

SEQ ID NO: 21 - protein, 178 aa, artificial sequence
  Lipocalin mutein
QDSTSDLIPAPPLSKVPLQQNFQDNQFHGKWYVVGRAGNDVLREDKDPEKMEATIYELKEDKSYNVTDVGFVQKRCMYVTHTFVPGSQPGEFTLGNIESYPGVTSALVRVVSTDYNQHAMVFVKLVKQNRELFEITLYGRTKELTSELKENFIRFSKSLGLPENHIVFPVPIDQCIDG

SEQ ID NO: 22 - protein, 178 aa, artificial sequence
  Lipocalin mutein
QDSTSDLIPAPPLSKVPLQQNFQDNQFHGKWYVVGRAANDVLRDDNDPEKMEATIYELKEDKSYNVTDVGFVQKKCMYVVHTFVPGSQPGEFTLGNIKSYPGVTSALVRVVSTNYNQHAMVFFKLVKQNRELFEVTLYGRTKELTSELKENFIRFSKSLGLPENHIVFPVPIDQCIDG

SEQ ID NO: 23 - protein, 178 aa, artificial sequence
  Lipocalin mutein
QDSTSDLIPAPPLSKVPLQQNFQDNQFHGKWYVVGRAGNDTLREDKDPEKMEATIYELKEDKSYNVTDVGLVQKKCMYVIHTFVPGSQPGEFTLGNIKSYPGVTSALVRVVSTNYNQHAMVFVKLVKQSRELFEITLYGRTKELTSELKENFIRFSKSLGLPENHIVFPVPIDQCIDG

SEQ ID NO: 24 - protein, 178 aa, artificial sequence
  Lipocalin mutein
QDSTSDLIPAPPLSKVPLQQNFQDNQFHGKWYVVGRAGNDTLRKDKDPEKMEATIYELKEDKSYNVTDVGFVQKKCMYVIHTFVPGSQPGEFTLGNIKSYPGVTSALVRVVSTNYNQHAMVFVKLVKQNRELFEITLYGRTKELTSELKENFIRFSKSLGLPENHIVFPVPIDQCIDG

SEQ ID NO: 25 - protein, 178 aa, artificial sequence
  Lipocalin mutein
QDSTSDLIPAPPLSKVPLQQNFQDNQFHGKWYVVGRAGNDALREDKDPEKMEATIYELKEDKSYNVTDVGFVQKKCMYVIHTFVPGSQPGEFTLGNIKSYPGVTSALIRVVNTNYNQHAMVFVKLVKQNRELFEITLYGRTKELTSELKENFIRFSKSLGLPENHIVFPVPIDQCIDG

SEQ ID NO: 26 - protein, 178 aa, artificial sequence
  Lipocalin mutein
QDSTSDLIPAPPLSKVPLQQNFQDNQFHGKWYVVGRAGNDALREDKDPEKMEATIYELKEDKSYNVTDVGLVQKKCMYVIHTFVPGGQPGEFTLGNIKSYPGVTSALVRVVSTNYNQHAMVFVKLVKQNRELFEITLYGRTKELTSELKENFIRFSKSLGLPENHIVFPVPIDQCIDG

SEQ ID NO: 27 - protein, 178 aa, artificial sequence
  Lipocalin mutein
QDSTSDLIPAPPLSKVPLQQNFQDNQFHGKWYVVGWAGNTLLREDKDPIKMQAKIYELKEDRSYNVTWVYFADKKCNYHIETFVPGSQPGEFTLGLIKSYPGETPILVRVVSTNYNQHAMVFFKGVNQNREIFEIILYGRTKELTNELKENFIRFSKSLGLPENHIVFPVPIDQCIDG

SEQ ID NO: 28 - protein, 178 aa, artificial sequence
  Lipocalin mutein
QDSTSDLIPAPPLSKVPLQQNFQDNQFHGKWYVVGWAGNTLLREDKDPIKMQAKIYELKEDRSYNVTWVYFAEKKCNYHIETFVPGSQPGEFTLGTIKSYPGETPILVRVVSTNYNQHAMVFFKGVNQNREIFEIILYGRTKELTNELKENFIRFSKSLGLPENHIVFPVPIDQCIDG

SEQ ID NO: 29 - protein, 178 aa, artificial sequence
  Lipocalin mutein
QDSTSDLIPAPPLSKVPLQQNFQDNQFHGKWYVVGWAGNTLLREDKDPIKMQAKIYELKEDRSYNVTWVYFADKKCNYHIETFVPGSQPGEFTLGTIKSYPGETPILVRVVSTNYNQHAMVFFKGVNQNREIFEIILYGRTKELTNELKENFIRFSKSLGLPENHIVFPVPIDQCIDG

SEQ ID NO: 30 - protein, 178 aa, artificial sequence
  Lipocalin mutein
QDSTSDLIPAPPLSKVPLQQNFQDNQFHGKWYVVGWAGNTLLREDKDPIKMQAKIYELKEDRSYNVTWVYFADKKCRYHIETFVPGSQPGEFTLGTIKSHPGETPILVRVVSTNYNQHAMVFFKGVNQNREIFEIILYGRTKELTNELKENFIRFSKSLGLPENHIVFPVPIDQCIDG

SEQ ID NO: 31 - protein, 178 aa, artificial sequence
  Lipocalin mutein
QDSTSDLIPAPPLSKVPLQQNFQDNQFHGKWYVVGWAGNTLLREDKDPIKMQAMIYELKEDKSYNVTWVYFADKKCNYHIETFVPGSQPGEFTLGTIKSYPGETPILVRVVSTNYNQHAMVFFKGVNQNREIFEIVLYGRTKELTNELKENFIRFSKSLGLPENHIVFPVPIDQCIDG

SEQ ID NO: 32 - protein, 178 aa, artificial sequence
  Lipocalin mutein
QDSTSDLIPAPPLSKVPLQQNFQDNQFHGKWYVVGWAGNTLLREDKDPIKMQAIIYELKEDRSYNVTWVYFSEKKCNYHIETFVPGSQPGEFTLGTIKSYPGETPILVRVMSTNYNQHAMVFFKGVNQNREIFEIILYGRTKELTNELKENFIRFSKSLGLPENHIVFPVPIDQCIDG

SEQ ID NO: 33 - protein, 177 aa, artificial sequence
  Lipocalin mutein
QDSTSDLIPAPPLSKVPLQQNFQDNQFQGKWYVVGRAGNDVLREDKDPEKMEATIYELKEDKSYNVTDVGFVQKRLMYVTHTFVPGSQPGEFTLGNIESYPGVTSALVRVVSTDYNQHAMVFVKLVKQNRELFEITLYGRTKELTSELKENFIRFSKSLGLPENHIVFPVPIDQIDG

SEQ ID NO: 34 - protein, 177 aa, artificial sequence
  Lipocalin mutein
QDSTSDLIPAPPLSKVPLQQNFQDNQFQGKWYVVGRAGNDVLREDKDPEKMEATIYELKEDKSYNVTDVGFVQKRYMYVTHTFVPGSQPGEFTLGNIESYPGVTSALVRVVSTDYNQHAMVFVKLVKQNRELFEITLYGRTKELTSELKENFIRFSKSLGLPENHIVFPVPIDQIDG

SEQ ID NO: 35 - protein, 178 aa, artificial sequence
  Lipocalin mutein
QDSTSDLIPAPPLSKVPLQQNFQDNQFQGKWYVVGWAGNTLLREDKDPIKMQAMIYELKEDKSYNVTWVYFADKKRNYHIETFVPGSQPGEFTLGTIKSYPGETPILVRVVSTNYNQHAMVFFKGVNQNREIFEIVLYGRTKELTNELKENFIRFSKSLGLPENHIVFPVPIDQFIDG

SEQ ID NO: 36 - protein, 178 aa, artificial sequence
  Lipocalin mutein
QDSTSDLIPAPPLSKVPLQQNFQDNQFQGKWYVVGWAGNTLLREDKDPIKMQAMIYELKEDKSYNVTWVYFADKKMNYHIETFVPGSQPGEFTLGTIKSYPGETPILVRVVSTNYNQHAMVFFKGVNQNREIFEIVLYGRTKELTNELKENFIRFSKSLGLPENHIVFPVPIDQYIDG

SEQ ID NO: 37 - protein, 178 aa, artificial sequence
  Lipocalin mutein
QDSTSDLIPAPPLSKVPLQQNFQDNQFQGKWYVVGWAGNTLLREDKDPIKMQAMIYELKEDKSYNVTWVYFADKKLNYHIETFVPGSQPGEFTLGTIKSYPGETPILVRVVSTNYNQHAMVFFKGVNQNREIFEIVLYGRTKELTNELKENFIRFSKSLGLPENHIVFPVPIDQWIDG

SEQ ID NO: 38 - protein, 178 aa, artificial sequence
  Lipocalin mutein
QDSTSDLIPAPPLSKVPLQQNFQDNQFQGKWYVVGWAGNTLLREDKDPIKMQAMIYELKEDKSYNVTWVYFADKKINYHIETFVPGSQPGEFTLGTIKSYPGETPILVRVVSTNYNQHAMVFFKGVNQNREIFEIVLYGRTKELTNELKENFIRFSKSLGLPENHIVFPVPIDQEIDG

SEQ ID NO: 39 - protein, 178 aa, artificial sequence
  Lipocalin mutein
QDSTSDLIPAPPLSKVPLQQNFQDNQFQGKWYVVGWAGNTLLREDKDPIKMQAMIYELKEDKSYNVTWVYFADKKVNYHIETFVPGSQPGEFTLGTIKSYPGETPILVRVVSTNYNQHAMVFFKGVNQNREIFEIVLYGRTKELTNELKENFIRFSKSLGLPENHIVFPVPIDQYIDG

SEQ ID NO: 40 - protein, 178 aa, artificial sequence
  Lipocalin mutein
QDSTSDLIPAPPLSKVPLQQNFQDNQFQGKWYVVGWAGNTLLREDKDPIKMQAMIYELKEDKSYNVTWVYFADKKRNYHIETFVPGSQPGEFTLGTIKSYPGETPILVRVVSTNYNQHAMVFFKGVNQNREIFEIVLYGRTKELTNELKENFIRFSKSLGLPENHIVFPVPIDQWIDG

SEQ ID NO: 41 - protein, 178 aa, artificial sequence
  Lipocalin mutein
QDSTSDLIPAPPLSKVPLQQNFQDNQFQGKWYVVGWAGNTLLREDKDPIKMQAMIYELKEDKSYNVTWVYFADKKNNYHIETFVPGSQPGEFTLGTIKSYPGETPILVRVVSTNYNQHAMVFFKGVNQNREIFEIVLYGRTKELTNELKENFIRFSKSLGLPENHIVFPVPIDQLIDG

SEQ ID NO: 42 - protein, 178 aa, artificial sequence
  Lipocalin mutein
QDSTSDLIPAPPLSKVPLQQNFQDNQFQGKWYVVGWAGNTLLREDKDPIKMQAMIYELKEDKSYNVTWVYFADKKRNYHIETFVPGSQPGEFTLGTIKSYPGETPILVRVVSTNYNQHAMVFFKGVNQNREIFEIVLYGRTKELTNELKENFIRFSKSLGLPENHIVFPVPIDQVIDG

SEQ ID NO: 43 - protein, 178 aa, artificial sequence
  Lipocalin mutein
QDSTSDLIPAPPLSKVPLQQNFQDNQFQGKWYVVGWAGNTLLREDKDPIKMQAMIYELKEDKSYNVTWVYFADKKKNYHIETFVPGSQPGEFTLGTIKSYPGETPILVRVVSTNYNQHAMVFFKGVNQNREIFEIVLYGRTKELTNELKENFIRFSKSLGLPENHIVFPVPIDQDIDG

SEQ ID NO: 44 - protein, 178 aa, artificial sequence
  Lipocalin mutein
QDSTSDLIPAPPLSKVPLQQNFQDNQFQGKWYVVGWAGNTLLREDKDPIKMQAMIYELKEDKSYNVTWVYFADKKFNYHIETFVPGSQPGEFTLGTIKSYPGETPILVRVVSTNYNQHAMVFFKGVNQNREIFEIVLYGRTKELTNELKENFIRFSKSLGLPENHIVFPVPIDQDIDG

SEQ ID NO: 45 - protein, 179 aa, artificial sequence
  Lipocalin mutein
GQDSTSDLIPAPPLSKVPLQQNFQDNQFQGKWYVVGWAGNTLLREDKDPIKMQAMIYELKEDKSYNVTWVYFADKKINYHIETFVPGSQPGEFTLGTIKSYPGETPILVRVVSTNYNQHAMVFFKGVNQNREIFEIVLYGRTKELTNELKENFIRFSKSLGLPENHIVFPVPIDQEIDG

SEQ ID NO: 46 - protein, 179 aa, artificial sequence
  Lipocalin mutein
GQDSTSDLIPAPPLSKVPLQQNFQDNQFHGKWYVVGRAGNDVLREDKDPEKMEATIYELKEDKSYNVTDVGFVQKRCMYVTHTFVPGSQPGEFTLGNIESYPGVTSALVRVVSTDYNQHAMVFVKLVKQNRELFEITLYGRTKELTSELKENFIRFSKSLGLPENHIVFPVPIDQCIDG

SEQ ID NO: 47 - protein, 179 aa, artificial sequence
  Lipocalin mutein
GQDSTSDLIPAPPLSKVPLQQNFQDNQFHGKWYVVGRAGNDVLREDKDPEKMEATIYELKEDKSYQVTDVGFVQKRCMYVTHTFVPGSQPGEFTLGNIESYPGVTSALVRVVSTDYNQHAMVFVKLVKQNRELFEITLYGRTKELTSELKENFIRFSKSLGLPENHIVFPVPIDQCIDD

SEQ ID NO: 48 - protein, 179 aa, artificial sequence
  Lipocalin mutein
GQDSTSDLKPAPPLSKVPLQQNFQDNQFHGKWYVVGRAGNDVLREDKDPEKMEATIYELKEDKSYNVTDVGFVQKRCMYVTHTFVPGSQPGEFTLGNIESYPGVTSALVRVVSTDYNQHAMVFVKLVKQNRELFEITLYGRTKELTSELKENFIRFSKSLGLPENHIVFPVPIDQCIDG

SEQ ID NO: 49 - protein, 179 aa, artificial sequence
  Lipocalin mutein
GQDSTSDLIHAPPLSKVPLQQNFQDNQFHGKWYVVGRAGNDVLREDKDPEKMEATIYELKEDKSYNVTDVGFVQKRCMYVTHTFVPGSQPGEFTLGNIESYPGVTSALVRVVSTDYNQHAMVFVKLVKQNRELFEITLYGRTKELTSELKENFIRFSKSLGLPENHIVFPVPIDQCIDG

SEQ ID NO: 50 - protein, 179 aa, artificial sequence
  Lipocalin mutein
GQDSTSDLKPAPPLSKVPLQQNFQDNQFHGKWYVVGRAGNDVLREDKDPEKMEATIYELKEDKSYQVTDVGFVQKRCMYVTHTFVPGSQPGEFTLGNIESYPGVTSALVRVVSTDYNQHAMVFVKLVKQNRELFEITLYGRTKELTSELKENFIRFSKSLGLPENHIVFPVPIDQCIDD

SEQ ID NO: 51 - protein, 179 aa, artificial sequence
  Lipocalin mutein
GQDSTSDLIHAPPLSKVPLQQNFQDNQFHGKWYVVGRAGNDVLREDKDPEKMEATIYELKEDKSYQVTDVGFVQKRCMYVTHTFVPGSQPGEFTLGNIESYPGVTSALVRVVSTDYNQHAMVFVKLVKQNRELFEITLYGRTKELTSELKENFIRFSKSLGLPENHIVFPVPIDQCIDD

SEQ ID NO: 52 - protein, 178 aa, artificial sequence
  Lipocalin mutein
QDSTSDLIPAPPLSKVPLQQNFQDNQFHGKWYVVGLAGNEVLREDKDPMKMWATIYELEEDKSYNVTIVMFLAKKCEYLFQTFVPGSQPGEFTLGDIKSSPGRTSGLVRVVSTNYNQHAMVFFKTVWQNREVFWITLYGRTKELTSELKENFIRFSKSLGLPENHIVFPVPIDQCIDG

SEQ ID NO: 53 - protein, 178 aa, artificial sequence
  Lipocalin mutein
QDSTSDLIPAPPLSKVPLQQNFQDNQFHGKWYVVGTAGNSILREDKDPQKMWATIYELKEDKSYNVTRVFFEGKKCRYVIETFVPGSQPGEFTLGKIKSAPGGTSILVRVVSTNYNQHAMVFFKVVWQNRELFWITLYGRTKELTSELKENFIRFSKSLGLPENHIVFPVPIDQCIDG

SEQ ID NO: 54 - protein, 178 aa, artificial sequence
  Lipocalin mutein
QDSTSDLIPAPPLSKVPLQQNFQDNQFHGKWYVVGVAGNGLLREDKDPLKMHATIYELKEDKSYNVTRVLFVRKKCRYYISTFVPGSQPGEFTLGRIKSEPGRTSFLVRVVSTNYNQHAMVFFKMVWQNREVFWITLYGRTKELTSELKENFIRFSKSLGLPENHIVFPVPIDQCIDG

SEQ ID NO: 55 - protein, 178 aa, artificial sequence
  Lipocalin mutein
QDSTSDLIPAPPLSKVPLQQNFQDNQFHGKWYVVGVAGNEMLREDKDPLKMLATIYELKEDKSYNVTRVMFEYKKCVYLIETFVPGSQPGEFTLGTIKSVPGLTSGLVRVVSTNYNQHAMVFFKRVWQNREVFWITLYGRTKELTSELKENFIRFSKSLGLPENHIVFPVPIDQCIDG

SEQ ID NO: 56 - protein, 178 aa, artificial sequence
  Lipocalin mutein
QDSTSDLIPAPPLSKVPLQQNFQDNQFHGKWYVVGAAGNSLLREDKDPMKMWATIYELKEDKSYNVTRVNFGGKKCSYLIETFVPGSQPGEFTLGSIKSRPGATSVLVRVVSTNYNQHAMVFFKLVTQNREVFWITLYGRTKELTSELKENFIRFSKSLGLPENHIVFPVPIDQCIDG

SEQ ID NO: 57 - protein, 178 aa, artificial sequence
  Lipocalin mutein
QDSTSDLIPAPPLSKVPLQQNFQDNQFHGKWYVVGLAGNEILREDKDPLKMWATIYELKEDKSYNVTRVQFGEKKCGYGIETFVPGSQPGEFTLGSIKSVPGGTSRLVRVVSTNYNQHAMVFFKFVWQNREVFWITLYGRTKELTSELKENFIRFSKSLGLPENHIVFPVPIDQCIDG

SEQ ID NO: 58 - protein, 178 aa, artificial sequence
  Lipocalin mutein
QDSTSDLIPAPPLSKVPLQQNFQDNQFHGKWYVVGLAGNRVLREDKDPQKMFATIYELKEDKSYNVTGVDFRTKKCLYSIGTFVPGSQPGEFTLGVIKSQPGWTSYLVRVVSTNYNQHAMVFFKTVWQNREVFWITLYGRTKELTSELKENFIRFSKSLGLPENHIVFPVPIDQCIDG

SEQ ID NO: 59 - protein, 178 aa, artificial sequence
  Lipocalin mutein
QDSTSDLIPAPPLSKVPLQQNFQDNQFHGKWYVVGLAGNEVLREDKDPMKMWATIYELEEDKSYNVTIVMPLAEKCEYLFQTFVPGSQPGEFTLGGIKSGPGRTSGLVRVVSTNYNQHAMVFFKVVWQNREVFWVTLYGRTKELTSELKENFIRFSKSLGLPENHIVFPVPIDQCIDG

SEQ ID NO: 60 - protein, 178 aa, artificial sequence
  Lipocalin mutein
QDSTSDLIPAPPLSKVPLQQNFQDNQFHGKWYVVGLAGNEVLREDKDPMKMWATIYELEEDKSYNVTIVMSLAKKCEYLFQTFVPGSQPGEFTLGDIKSSPGRTSGLVRVVSTNYNQHAMVFFKVVWQNREVFWITLYGRTKELTSGLKENFIRFSKSLGLPENHIVFPVPIDQCIDG

SEQ ID NO: 61 - protein, 178 aa, artificial sequence
  Lipocalin mutein
QDSTSDLIPAPPLSKVPLQQNFQDNQFHGKWYVVGLAGNEILREDKDPMKMWATIYELEEDRSYNVTIVMFLAKKCEYLFQTFVPGSQPGEFTLGDIKSSPGRTSGLVRVVSTNYNQHAMVFFKVVWQNREVFWITLYGRTKELTPELKENFIRFSKSLGLPENHIVFPVPIDQCIDG

SEQ ID NO: 62 - protein, 178 aa, artificial sequence
  Lipocalin mutein
QDSTSDLIPAPPLSKVPLQQNFQDNQFHGKWYVVGLAGNEILREDKDPMKMWATIYELEEDKSYNVTIVMFLAKKCEYLFQTFVPGSQPGEFTLGDIKSSPGRTSGLVRVVSTNYNQHAMVFFKVVWQNREVFWITLYGRTKELTSGLKENFIRFSKSLGLPENHIVFPVPIDQCIDG

SEQ ID NO: 63 - protein, 178 aa, artificial sequence
  Lipocalin mutein
QDSTSDLIPAPPLSKVPLQQNFQDNQFHGKWYVVGLAGNEVLREDKDPMKMWATIYELEEDKSYNVTIVMFLAEECEYLFQTFVPGSQPGEFTLGDIKSSPGRTSGLVRVVSTNYNQHAMVFFKVVWQNREVFWITLYGRTKELTSELKKNFIRFSKSLGLPENHIVFPVPIDQCIDG

SEQ ID NO: 64 - protein, 178 aa, artificial sequence
  Lipocalin mutein
QDSTSDLIPAPPLSKVPLQQNFQDNQFHGKWYVVGLAGNEVLREDKDPMKMWATIYELEEDKSYNVTIVMPLAEKCEYLFQTFVPGCQPGEFTLGGIKSGPGRTSGLVRVVSTNYNQHAMVFFKVVWQNREVFWVTLYGRTKELTSELKENFIRFSKSLGLPENHIVFPVPIDQCIDG

SEQ ID NO: 65 - protein, 178 aa, artificial sequence
  Lipocalin mutein
QDSTSDLIPAPPLSKVPLQQNFQDNQFHGKWYVVGLAGNEVLREDKDPMKMWATIYELEEDKSYNVTIVMFLAKKCEYLFQTFVPGCQPGEFTLGDIKSSPGRTSGLVRVVSTNYNQHAMVFFKTVWQNREVFWITLYGRTKELTSELKENFIRFSKSLGLPENHIVFPVPIDQCIDG

SEQ ID NO: 66 - protein, 152 aa, artificial sequence
  Lipocalin mutein
ASDEEIQDVSGTWYLKAMTVDRFKIASWPRSVTPMTLTTLEGGNLEAKVTMNWWGRSQEVKAVLERTDEPGKYTAQGDRHVAYIIRSHVKDHYIFYSEGNLQGETVPGVWLVGRDPKNNLEALEDFEKAAGARGLSTESILIPRQSETSSPG

SEQ ID NO: 67 - protein, 152 aa, artificial sequence
  Lipocalin mutein
ASDEEIQDVSGTWYLKAMTVDRFKIASWPRSVTPMTLTTLEGGNLEAKVTMNWWGRSQEVKAVLERTDEPGKYTAQGDRHVAYIIRSPVKDHYIFYSEGNLQGETVPGVWLVGRDPKNNLEALEDFEKAAGARGLSTESILIPRQSETSSPG

SEQ ID NO: 68 - protein, 152 aa, artificial sequence
  Lipocalin mutein
ASDEEIQDVSGTWYLKAMTVDFSRGDAIWTSVTPMTLTTLEGGNLEAKVTMMYAGRSQEVKAVLERTDEPGKYTTMGGSHVAYIIRSPVKDHYIFYSEGPHQGAPVPGVWLVGRDPKNNLEALEDFEKAAGARGLSTESILIPRQSETSSPG

SEQ ID NO: 69 - protein, 152 aa, artificial sequence
  Lipocalin mutein
ASDEEIQDVSGTWYLKAMTVDFSRGDAIWTSVTPMTLTTLEGGNLEAKVTMMYAGRSQEVKAVLEKTDEPGKYTAMGGSHVAYITRSHVKDHYIFYSEGPHQGAPVPGVWLVGRDPKNNLEALEDFEKAAGARGLSTESILIPRQSETSSPG

SEQ ID NO: 70 - protein, 152 aa, artificial sequence
  Lipocalin mutein
ASDEEIQDVSGTWYLKAMTVDFSRGDAIWTSVTPMTLTILEGGNLEAKVTMMYAGRSQGVKVVLEKTDEPGKYTAMGGSQVAYIIRSHVKDHYIFYPEGPYQGAPVPGVWLVGRDPKNNLEALEDFEKAAGARGLSTESILIPRQSETSSPG

SEQ ID NO: 71 - protein, 152 aa, artificial sequence
  Lipocalin mutein
ASDEEIQDVSGTWYLKAMTVDFSRGDAIWTSVTPMTLTTLEGGNLEAKVTMMYAGRSQEVKAVLEKTDEPGKYTTMGGSHVAYIIRSHVKDHYIFYSEGPYQGAPVPGVWLVGRDPKNNLEALEDFEKAAGARGLSTESILIPRQSETSSPG

SEQ ID NO: 72 - protein, 152 aa, artificial sequence
  Lipocalin mutein
ASDEEIQDVSGTWYLKAMTVDFSRGDAIWTSVTPMTLTTLEGGNLEAKVTMMYAGRSQEVKAVLEKTDEPGKYTASGGSHVAYIIRSHVKDHYIFYSEGPHQGAPVPGVWLVGRDPKNNLEALEDFEKAAGARGLSTESILIPRQSETSSPG

SEQ ID NO: 73 - protein, 152 aa, artificial sequence
  Lipocalin mutein
ASDEEIQDVSGTWYLKAMTVDFSRGDAIWTSVTPMTLTALEGGGLEAKVTMMYAGRSQEVKVVLEKTDEPGKYTAMGGSHGAYITRSHVKDHYIFYSEGPYRGAPVPGVWLVGRDPKNNLEALEDFEKAAGARGLSTESILIPRQSETSSPG

SEQ ID NO: 74 - protein, 152 aa, artificial sequence
  Lipocalin mutein
ASDEEIQDVSGTWYLKAMTVDFSRGDAIWTSVTPMTLTTLGGGNLEAKVTMMYAGRSQEVKAVLVKTDEPGKYTAMGSSHVAYIIRSHVKDHYIFYSEGPYQGAPVPGVWLVGRDPKNNLEALEDFEKAAGARGLSTESILIPRQSETSSPG

SEQ ID NO: 75 - protein, 152 aa, artificial sequence
  Lipocalin mutein
ASDEEIQDVSGTWYLKAMTVDFSRGDAIWTSVTPMTLTTLEGGNLEAKVTMMYAGRSQEVKAVLEKTDEPGKYTVMGGSHVASIIRSHVKDHYIFYPEGPYQGAPVPGVWLVGRDPKNNLEALEDFEKAAGARGLSTESILIPRQSETSSPG

SEQ ID NO: 76 - protein, 152 aa, artificial sequence
  Lipocalin mutein
ASDEEIQDVSGTWYLKAMTVDFSRGDAIWTSVTPMTLTTLEGGNLEAKVTMMYAGRSQEVKAVLEKTDEPGKYTAMGGSHVAYILRSHVKDHYIFYSEGPHQGAPVPGVWLVGRDPKNNLEALEDFEKAAGARGLSTESILIPRQSETSSPG

SEQ ID NO: 77 - protein, 152 aa, artificial sequence
  Lipocalin mutein
ASDEEIQDVSGTWYLKAMTVDRFKIASWPRSVTPMTLTTLGGGNLEAKVTMNWWGRSQEVKAVLEKTDEPGKYTAQGGSHVEHIIRSHVKDHYIFYSKGNLQGEPVPGVWLVGRDPKNNLEALEDFEKAAGARGLSTESILIPRQSETSSPG

SEQ ID NO: 78 - protein, 152 aa, artificial sequence
  Lipocalin mutein
ASDEEIQDVSGTWYLKAMTVDRFKIASWPRSVTPMTLTTLEGGNLEAKVTMNWWGRSQEVKAVLEKTDEPGKYTAQGGSHVEYIIRSHVKDHYIFYSEGNLQGEPVPGVWLVGRDPKNNLEALEDFEKAAGARGLSTESILIPRQSETSSPG

SEQ ID NO: 79 - protein, 152 aa, artificial sequence
  Lipocalin mutein
ASDEEIQDVSGTWYLKAMTVDRFKIASWPRSVTPMTLTTLEGGNLEAKVTMNWWGRSQDVKAVLGKTDEPGKYTAQGDSHVSYIIRSHVKDHYILYSVGNLQGETVPGVWLVGRDPKNNLEALEDFEKAAGARGLSTESILIPRQSETSSPG

SEQ ID NO: 80 - protein, 152 aa, artificial sequence
  Lipocalin mutein
ASDEEIQDVSGTWYLKAMTVDRFKIASWPRSVTPMTLTTLEGGNLEAKVTMNWWGRSQEVKAVLEKTDEPGKYTAVGGSHVAYIIRSHVKDHYIFYSEGKLQGEPVPGVWLVGRDPKNNLEALEDFEKAAGARGLSTESILIPRQSETSSPG

SEQ ID NO: 81 - protein, 152 aa, artificial sequence
  Lipocalin mutein
ASDEEIQDVSGTWYLKAMTVDRFKIGSWPRSVTPMTLTTLEGGNLEAKVTMNWRGRSQEVKAVLEKTDEPGKYTAQGGSHVAYIIRSHVKDHYIFYSEGKLQGEPVPGVWLVGRDPKNNLEALEDFEKAAGARGLSTESILIPRQSETSSPG

SEQ ID NO: 82 - protein, 152 aa, artificial sequence
  Lipocalin mutein
ASDEEIQDVSGTWYLKAMTVDRFKIGAWPRSVTPMTLTTLEGGNLEAKVTMNWWGRSQEVKAVLEKTDEPGKYTATGGSHVAYIIRSHVKDHYIFYSEGKLQGEPVPGVWLVGRDPKNNLEALEDFEKAAGARGLSTESILIPRQSETSSPG

SEQ ID NO: 83 - protein, 152 aa, artificial sequence
  Lipocalin mutein
ASDEEIQDVSGTWYLKAMTVDFSRGDAIWTSVTPMTLTTLEGGNLEAKVTMMYAGRSQEVKAVLERTDEPGKYTVMGGSHVAYIIRSPVKDHYIFYSEGPGQGAPVPGVWLVGRDPKNNLEALEDFEKAAGARGLSTESILIPRQSETSSPG

SEQ ID NO: 84 - protein, 152 aa, artificial sequence
  Lipocalin mutein
ASDEEIQDVSGTWYLKAMTVDFSRGDAIWTSVTPMTLTTLEGGNLEAKVTMMYAGRSQEVKAVLERTDEPGKYTVMGGSHVAYIIRSPVKDHYIFYSEGPRQGAPVPGVWLVGRDPKNNLEALEDFEKAAGARGLSTESILIPRQSETSSPG

SEQ ID NO: 85 - protein, 152 aa, artificial sequence
  Lipocalin mutein
ASDEEIQDVSGTWYLKAMTVDFSRGDAIWTSVTPMTLTTLEGGNLEAKVTMMYAGRSQEVKAVLERTDEPGKYTTMGGSHVAYIIRSPVKDHYIFYSEGPAQGAPVPGVWLVGRDPKNNLEALEDFEKAAGARGLSTESILIPRQSETSSPG

SEQ ID NO: 86 - protein, 152 aa, artificial sequence
  Lipocalin mutein
ASDEEIQDVSGTWYLKAMTVDFSRGDAIWTSVTPMTLTTLEGGNLEAKVTMMYAGRSQEVKAVLERTDEPGKYTVMGGSHVAYIIRSPVKDHYIFYSEGPDQGAPVPGVWLVGRDPKNNLEALEDFEKAAGARGLSTESILIPRQSETSSPG

SEQ ID NO: 87 - protein, 152 aa, artificial sequence
  Lipocalin mutein
ASDEEIQDVSGTWYLKAMTVDFSRGDAIWTSVTPMTLTTLEGGNLEAKVTMMYAGRSQEVKAVLERTDEPGKYTMMGGSHVAYIIRSPVKDHYIFYSEGPVQGAPVPGVWLVGRDPKNNLEALEDFEKAAGARGLSTESILIPRQSETSSPG

SEQ ID NO: 88 - protein, 152 aa, artificial sequence
  Lipocalin mutein
ASDEEIQDVSGTWYLKAMTVDFSRGDAIWTSVTPMTLTTLEGGNLEAKVTMMYAGRSQEVKAVLERTDEPGKYTVMGGSHVAYIIRSPVKDHYIFYSEGPPQGAPVPGVWLVGRDPKNNLEALEDFEKAAGARGLSTESILIPRQSETSSPG

SEQ ID NO: 89 - protein, 152 aa, artificial sequence
  Lipocalin mutein
ASDEEIQDVSGTWYLKAMTVDFSRGDAIWTSVTPMTLTTLEGGNLEAKVTMMYAGRSQEVKAVLERTDEPGKYTVMGGSHVAYIIRSPVKDHYIFYSEGPKQGAPVPGVWLVGRDPKNNLEALEDFEKAAGARGLSTESILIPRQSETSSPG

SEQ ID NO: 90 - protein, 152 aa, artificial sequence
  Lipocalin mutein
ASDEEIQDVSGTWYLKAMTVDFSRGDAIWTSVTPMTLTTLEGGNLEAKVTMMYAGRSQEVKAVLERTDEPGKYTTMGGSHVAYIIRSPVKDHYIFYSEGPRQGAPVPGVWLVGRDPKNNLEALEDFEKAAGARGLSTESILIPRQSETSSPG

SEQ ID NO: 91 - protein, 152 aa, artificial sequence
  Lipocalin mutein
ASDEEIQDVSGTWYLKAMTVDFSRGDAIWTSVTPMTLTTLEGGNLEAKVTMMYAGRSQEVKAVLERTDEPGKYTVMGGSHVAYIIRSPVKDHYIFYSEGPLQGAPVPGVWLVGRDPKNNLEALEDFEKAAGARGLSTESILIPRQSETSSPG

SEQ ID NO: 92 - protein, 178 aa, artificial sequence
  Lipocalin mutein
QDSTSDLIPAPPLSKVPLQQNFQDNQFHGKWYVVGLAGNRLLRDDQHPMDMYATIYELKGDKSYNVTSVISSHKKCLYPIATFVPGSQPGEFTLGNIKSYGDKWSYLVRVVSTDYNQYAVVFFKHADTNYESFSITIYGRTKELASELKENFIRFSKSLGLPENHIVFPVPIDQCIDG

SEQ ID NO: 93 - protein, 178 aa, artificial sequence
  Lipocalin mutein
QDSTSDLIPAPPLSKVPLQQNFQDNQFHGKWYVVGLAGNRILRQDQHPMLMYATIYELKGDKSYNVTSVISSHKKCLYPIATFVPGSQPGEFTLGNIKSYGDKVSYLVRVVSTDYNQYAVVFFKHADTNYESFSITIYGRTKELASELKENFIRFSKSLGLPENHIVFPVPIDQCIDG

SEQ ID NO: 94 - protein, 178 aa, artificial sequence
  Lipocalin mutein
QDSTSDLIPAPPLSKVPLQQNFQDNQFHGKWYVVGLAGNRILRDDQHPMPMYATIYELKGDKSYNVTSVISSHKKCLYPIATFVPGSQPGEFTLGNIKSYGDKWSYLVRVVSTDYNQYAVVFFKHADSNYESFSITIYGRTKELASELKENFIRFSKSLGLPENHIVFPVPIDQCIDG

SEQ ID NO: 95 - protein, 178 aa, artificial sequence
  Lipocalin mutein
QDSTSDLIPAPPLSKVPLQQNFQDNQFHGKWYVVGLAGNRILRSDQHPMRMYATIYELKGDKSYNVTSVISSHKKCLYTIATFVPGSQPGEFTLGNIKSYGDKWSYLVRVVSTDYNQYAVVFFKHADTNYESFSITIYGRTKELASELKENFIRFSKSLGLPENHIVFPVPIDQCIDG

SEQ ID NO: 96 - protein, 178 aa, artificial sequence
  Lipocalin mutein
QDSTSDLIPAPPLSKVPLQQNFQDNQFHGKWYVVGLAGNRILRSDQHPMPMYATIYELKGDKSYNVTSVISSHKKCLYSIATFVPGSQPGEFTLGNIKSYGDKESYLVRVVSTDYNQYAVVFFKYASDNDESFSITIYGRTKELASELKENFIRFSKSLGLPENHIVFPVPIDQCIDG

SEQ ID NO: 97 - protein, 178 aa, artificial sequence
  Lipocalin mutein
QDSTSDLIPAPPLSKVPLQQNFQDNQFHGKWYVVGLAGNRILRLDQHPMPMYATIYELKGDKSYNVTSVISSHKKCLYPIATFVPGSQPGEFTLGNIRSYGDKTSYLVRVVSTDYNQYAVVFFKHADTNYESFSITIYGRTKELASELKENFIRFSKSLGLPENHIVFPVPIDQCIDG

SEQ ID NO: 98 - protein, 178 aa, artificial sequence
  Lipocalin mutein
QDSTSDLIPAPPLSKVPLQQNFQDNQFHGKWYVVGLAGNRILRDDQHPMNMYATIYELKGDKSYNVTSVISSHKKCLYTIATFVPGSQPGEFTLGNIKSYGDKTSYLVRVVSTDYNQYAVVFFKLAEDNAESFAITIYGRTKELASELKENFIRFSKSLGLPENHIVFPVPIDQCIDG

SEQ ID NO: 99 - protein, 152 aa, artificial sequence
  Lipocalin mutein
ASDEEIQDVSATWYLKAMTVDFWCSGVHEESVTPMTLTTLEGGNLEAKVTMDIGGFLQEVKAVLEKTDEPGKYTADGGKHVAYIIRSHVKDHYIFYSEGDCPGLPVPGVWLVGRDPKNNLEALEDFEKAAGARGLSTESILIPRQSETSSPG

SEQ ID NO: 100 - protein, 151 aa, artificial sequence
  Lipocalin mutein
ASDEEIQDVSATWYLKAMTVDFWCSGIHDESVTPMTLTTLEGGNLEAKVTMDIAGFLQEVKAVLEKTDEPGKYTADGGKHVAYIIRSHVKDHYIFYSEGDCPGPVPGVWLVGRDPKNNLEALEDFEKAAGARGLSTESILIPRQSETSSPG

SEQ ID NO: 101 - protein, 152 aa, artificial sequence
  Lipocalin mutein
ASDEEIQDVSATWYLKAMTVDFWCSGIHEESVTPMTLTTLEGGNLEAKVTMDIRGFLQEFKAVLEKTDEPGKYTADGGKHVAYIIRSHVKDHYIFYSEGDCPDSPVPGVWLVGRDPKNNLEALEDFEKAAGARGLSTESILIPRQSETSSPG

SEQ ID NO: 102 - protein, 151 aa, artificial sequence
  Lipocalin mutein
ASDEEIQDVSATWYLKAMTVDFWCSGIHEESVTPMTLTTLEGGNLEAKVTMDIEGFLQEFKAVLEKTDEPGKYTADGGKHVAYIIRSHVKDHYIFYSEGDCPGPVPGVWLVGRDPKNNLEALEDFEKAAGARGLSTESILIPRQSETSSPG

SEQ ID NO: 103 - protein, 152 aa, artificial sequence
  Lipocalin mutein
ASDEEIQDVSGTWYLKAMTVDYGCNHPSIWSVTPMTLTTLEGGNLEAKVTMYWEGSRQEDKAVLEKTDEPGKYTADGGKHVAYIIRSHVKDHYIFYSEGICEGAPVPGVWLVGRDPKNNLEALEDFEKAAGARGLSTESILIPRQSETSSPG

SEQ ID NO: 104 - protein, 151 aa, artificial sequence
  Lipocalin mutein
ASDEEIQDVSGTWYLKAMTVDFWCSGIHEESVTPMTLTTLEGGNLEAKVTMDIEGFLQEFKAVLEKTDEPGKYTADGGKHVAYIIRSRVKDHYIFYSEGDCPGPVPGVWLVGRDPKNNLEALEDFEKAAGARGLSTESILIPRQSETSSPG

SEQ ID NO: 105 - protein, 152 aa, artificial sequence
  Lipocalin mutein
ASDEEIQDVSGTWYLKAMTVDWQCTWDDDPSVTPMTLTTLEGGNLEAKVTMPIFGLWQEEKAVLEKTDEPGKYTADGGKHVAYIIRSHVKDHYIFYSEGACYGQPVPGVWLVGRDPKNNLEALEDFEKAAGARGLSTESILIPRQSETSSPG

SEQ ID NO: 106 - protein, 152 aa, artificial sequence
  Lipocalin mutein
ASDEEIQDVSGTWYLKAMTVDWQCTWADEPSVTPMTLTTLEGGNLEAKVTIPTFGLAEEEKAVLEKTDEPGKYTADGGKHVAYIIRSHVKDHYIFYSEGRCWGRPVPGVWLVGRDPKNNLEALEDFEKAAGARGLSTESILIPRQSETSSPG

SEQ ID NO: 107 - protein, 152 aa, artificial sequence
  Lipocalin mutein
ASDEEIQDVSGTWYLKAMTVDWVCAFDDDPSVTPMTLTTLEGGNLEAKVTIPTFGLYEEEKAVLEKTDEPGKYTADGGKHVAYIIRSHVKDHYIFYSEGACHGHPVPGVWLVGRDPKNNLEALEDFEKAAGARGLSTESILIPRQSETSSPG

SEQ ID NO: 108 - protein, 178 aa, artificial sequence
  Lipocalin mutein
QDSTSDLIPAPPLSKVPLQQNFQDNQFHGKWYVVGMAGNLMLREDKDPTKMSATIYELKEDKSYNVTWVDFRFKKCKYQIGTFVPGSQPGEFTLGGIKSMPGMTSFLVRVVSTNYNQHAMVFFKYVYQNREYFEITLYGRTKELTSELKENFIRFSKSLGLPENHIVFPVPIDQCIDG

SEQ ID NO: 109 - protein, 178 aa, artificial sequence
  Lipocalin mutein
QDSTSDLIPAPPLSKVPLQQNFQDNQFHGKWYVVGLAGNTILREDKDPDKMEATIYELKEDKSYNVTSVDFIMKKCWYFITTFVPGSQPGEFTLGHIKSMPGMTSFLVRVVSTNYNQHAMVFFKYVYQNREFFEITLYGRTKELTSELKENFIRFSKSLGLPENHIVFPVPIDQCIDG

SEQ ID NO: 110 - protein, 178 aa, artificial sequence
  Lipocalin mutein
QDSTSDLIPAPPLSKVPLQQNFQDNQFHGKWYVVGEAGNLLLREDKDPRKMTATIYELKEDKSYNVTRVEFGVKTYKYQIGTFVPGSQPGEFTLGGIKSMPGMTSFLVRVVSTNYNQHAMVFFKYVYQNREYFEITLYGRTKELTSELKENFIRFSKSLGLPENHIVFPVPIDQAIDG

SEQ ID NO: 111 - protein, 178 aa, artificial sequence; identical to SEQ ID NO: 110
  Lipocalin mutein

SEQ ID NO: 112 - protein, 178 aa, artificial sequence
  Lipocalin mutein
QDSTSDLIPAPPLSKVPLQQNFQDNQFHGKWYVVGEAGNLLLREDKDPRKMTATIYELKEDKSYDVTRVEFGAKTYKYQIGTFVPGSQPGEFTLGGIESMPGMTSFLVRVVSTNYNQHAIVFFKYVYQNREYFEITLYGRTKELTSELKENFIRFSKSLGLPENHIVFPVPIDQAIDG

SEQ ID NO: 113 - protein, 178 aa, artificial sequence
  Lipocalin mutein
QDSTSDLIPAPPLSKVPLQQNFQDNQFHGKWYVVGEAGNLILREDKDPRKMTATIYELKEDKSYDVTRVEFGVKTRKYRIGTFVPGSQPGEFTLGGIKSMPGMTSFLVRVVSTNYNQHAMVFFKYVYQNREYFEITLYGRTKELTSELKENFIRFSKSLGLPENHIVFPVPIDQAIDG

SEQ ID NO: 114 - protein, 178 aa, artificial sequence
  Lipocalin mutein
QDSTSDLIPAPPLSKVPLQQNFQDNQFHGKWYVVGEAGNLLLREDKDPRKMTATIYELREDKSYDVTRVEFGVKTYKYQIGTFVPGSQPGEFTLGGIKSMPGMTSFLVRVVSTDYNQHAMVFFKYVYQNREYFEITLYGRTKELTSELKENFIRFSKSLGLPENHIVFPVPIDQAIDG

SEQ ID NO: 115 - protein, 178 aa, artificial sequence
  Lipocalin mutein
QDSTSDLIPAPPLSKVPLQQNFQDNQFHGKWYVVGNAGNGWLREDKDPIKMMATIYELKEDKSYNVTVVQFWDKKCLYQIQTFVPGSQPGEFTLGHIKSKPGHTSHLVRVVSTNYNQHAMVFFKRVWQNREWFDITLYGRTKELTSELKENFIRFSKSLGLPENHIVFPVPIDQCIDG

SEQ ID NO: 116 - protein, 178 aa, artificial sequence
  Lipocalin mutein
QDSTSDLIPAPPLSKVPLQQNFQDNQFHGKWYVVGTAGNGFLREDKDPLKMWATIYELKEDKSYNVTSVWFALKKCYYDIGTFVPGSQPGEFTLGIIKSEPGHTSQLVRVVSTNYNQHAMVFFKWVNQNRENFQITLYGRTKELTSELKENFIRFSKSLGLPENHIVFPVPIDQCIDG

SEQ ID NO: 117 - protein, 178 aa, artificial sequence
  Lipocalin mutein
QDSTSDLIPAPPLSKVPLQQNFQDNQFHGKWYVVGWAGNTTLREDKDPPKMPATIYELKEDKSYNVTDVQFSEKKCSYSIITFVPGSQPGEFTLGGIKSNPGKTSHLVRVVSTNYNQHAMVFFKYVAQNREGFNITLYGRTKELTSELKENFIRFSKSLGLPENHIVFPVPIDQCIDG

SEQ ID NO: 118 - protein, 178 aa, artificial sequence
  Lipocalin mutein
QDSTSDLIPAPPLSKVPLQQNFQDNQFHGKWYVVGFAGNNRLREDKDPPKMMATIYELKEDKSYNVTDVTFEAKKCRYRIITFVPGSQPGEFTLGYIKSKPGPTSFLVRVVSTNYNQHAMVFFKSVTQNREWFGITLYGRTKELTSELKENFIRFSKSLGLPENHIVFPVPIDQCIDG

SEQ ID NO: 119 - protein, 178 aa, artificial sequence
  Lipocalin mutein
QDSTSDLIPAPPLSKVPLQQNFQDNQFHGKWYVVGLAGNGWLREDKDPVKMMATIYELKEDKSYNVTVVDFELKKCRYMIETFVPGSQPGEFTLGDIKSFPGWTSQLVRVVSTNYNQHAMVFFKGVYQNREWFHITLYGRTKELTSELKENFIRFSKSLGLPENHIVFPVPIDQCIDG

SEQ ID NO: 120 - protein, 178 aa, artificial sequence
  Lipocalin mutein
QDSTSDLIPAPPLSKVPLQQNFQDNQFHGKWYVVGVAGNFFLREDKDPAKMPATIYELKEDKSYNVTEVWFLNKKCQYEIHTFVPGSQPGEFTLGYIKSYPGYTSHLVRVVSTNYNQHAMVFFKVVHQNRDKFWITLYGRTKELTSELKENFIRFSKSLGLPENHIVFPVPIDQCIDG

SEQ ID NO: 121 - protein, 178 aa, artificial sequence
  Lipocalin mutein
QDSTSDLIPAPPLSKVPLQQNFQDNQFHGKWYVVGWAGNTTLREDKDPPKMPATIYELKEDKSYNVTDVQFPEKKCIYSTITFVPGSQPGEFTLGGIKSSPGQTSHLVRVVSTNYNQHAMVFFKYVIQNREGFNITLYGRTKELTSELKENFIRFSKSLGLPENHIVFPVPIDQCIDG

SEQ ID NO: 122 - protein, 178 aa, artificial sequence
  Lipocalin mutein
QDSTSDLIPAPPLSKVPLQQNFQDNQFHGKWYVVGWAGNTTLREDKDPPKMPATIYELKEDKSYNVTDVQFPDKKCIYSIITFVPGSQPGEFTLGGIKSNPGDTSHLVRVVSTNYNQHAMVFFKYVVQNREGFNITLYGRTKELTSELKENFIRFSKSLGLPENHIVFPVPIDQCIDG

SEQ ID NO: 123 - protein, 178 aa, artificial sequence
  Lipocalin mutein
QDSTSDLIPAPPLSKVPLQQNFQDNQFHGKWYVVGWAGNTTLREDKDPPKMPATIYELKEDKSYNVTDVQFPEKKCTYSIITFVPGSQPGEFTLGDIKSNPGETSHLVRVMSTNYNQHAMVFFKYVDQNREGFNITLYGRTKELTSELKENFIRFSKSLGLPENHIVFPVPIDQCIDG

SEQ ID NO: 124 - protein, 178 aa, artificial sequence
  Lipocalin mutein
QDSTSDLIPAPPLSKVPLQQNFQDNQFHGKWYVVGWAGNTTLREDKDPPKMPATIYELKEDKSYNVTDVQFPDKKCVYSIITFVPGSQPGEFTLGGIKSNPGNTSHLVRVVSTNYNQHAMVFFKYVVQNREGFNITLYGRTKELTSELKENFIRFSKSLGLPENHIVFPVPIDQCIDG

SEQ ID NO: 125 - protein, 178 aa, artificial sequence
  Lipocalin mutein
QDSTSDLIPAPPLSKVPLQQNFQDNQFHGKWYVVGLAGNGWLREDKDPLKMMATIYELKEDKSYNVTVVDFELKKCRYMIETFVPGSQPGEFTLGDIKSSPGWTSQLVRVVSTNYNQHAMVFFKGVYQNREWFHITLYGRTKELTSELKENFIRFSKSLGLPENHIVFPVPIDQCIDG

SEQ ID NO: 126 - protein, 178 aa, artificial sequence
  Lipocalin mutein
QDSTSDLIPAPPLSKVPLQQNFQDNQFHGKWYVVGWAGNTTLREDKDPPKMPAVIYELKEDKSYNVTDVQFPEKECIYSTITFVPGSQPGEFTLGGIKSSPGQTSHLVRVVSTNYNQHAMVFFKYVTQNREGFNITLYGRTKELTSELKENFIRFSKSLGLPENHIVFPVPIDQCIDG

SEQ ID NO: 127 - protein, 178 aa, artificial sequence
  Lipocalin mutein
QDSTSDLIPAPPLSKVPLQQNFQDNQFHGKWYVVGLAGNGWLREDEDPLKMMAAVYELREDKSYNVTVVDFELEECRYMTETFVPGNQPGEFTLGDIKSSPGWTSQLVRVVSTNYNQHAMVFFKGVYQNREWFHITLYGRTKELTSELKENFIRFSKSLGLPENHIVFPVPIDQCIDG

SEQ ID NO: 128 - protein, 178 aa, artificial sequence
  Lipocalin mutein
QDSTSDLIPAPPLSKVPLQQNFQDNQFQGKWYVVGWAGDTTLREDKDPPKMPAVIYELKEDKSYDVTDVQFPEKECIYSTITFVPGSQPGEFTLGGIKSSPGQTSHLVRVVSTNYNQHAMVFFKYVTQNREGFNIALYGRTKELTSELKENFIRFSKSLGLPENHIVFPVPIDQCIDG

SEQ ID NO: 129 - protein, 178 aa, artificial sequence
  Lipocalin mutein
QDSTSDLIPAPPLSKVPLQQNFQDNQFQGKWYVVGWAGNTALREDKDPPKMPAVIYELKEDKSYDVTDVQFPEKECIYSTITFVPGSQPGEFTLGGIKSSPGQTSHLVRVVSTNYNQHAMVFFKYVTQNREGFNIALYGRTKELTSELKENFIRFSKSLGLPENHIVFPVPIDQCIDG

SEQ ID NO: 130 - protein, 178 aa, artificial sequence
  Lipocalin mutein
QDSTSDLIPAPPLSKVPLQQNFQDNQFHGKWYVVGLAGNGWLREDEDPLKMMAAVYELREDKSYDVTVVDFELEECRYMTETFVPGNQPGEFTLGDIKSSPGWTSQLVRVVSTNYNQHAMVFFKGVYQNREWFHITLYGRTKELTSELKENFIRFSKSLGLPENHIVFPVPIDQCIDG

SEQ ID NO: 131 - protein, 178 aa, artificial sequence
  Lipocalin mutein
QDSTSDLIPAPPLSKVPLQQNFQDNQFHGKWYVVGLAGNGWLREDEDPLKMMAAVYELREDKSYQVTVVDFELEECRYMTETFVPGNQPGEFTLGDIKSSPGWTSQLVRVVSTNYNQHAMVFFKGVYQNREWFHITLYGRTKELTSELKENFIRFSKSLGLPENHIVFPVPIDQCIDG

SEQ ID NO: 132 - protein, 178 aa, artificial sequence
  Lipocalin mutein
QDSTSDLIPAPPLSKVPLQQNFQDNQFHGKWYVVGVAGNEVLREDKDPGKMPATIYELKEDKSYNVTEVRFHNKKCNYSIETFVPGSQPGEFTLGNIKSNPGQTSMLVRVVSTNYNQHAMVFFKKVKQNREGFWITLYGRTKELTSELKENFIRFSKSLGLPENHIVFPVPIDQCIDG

SEQ ID NO: 133 - protein, 178 aa, artificial sequence
  Lipocalin mutein
QDSTSDLIPAPPLSKVPLQQNFQDNQFHGKWYVVGLAGNTILREDKDPGKMNATIYELKEDKSYNVTDVRFIMKKCHYYIETFVPGSQPGEFTLGIIKSNPGTTSQLVRVVSTNYNQHAMVFFKIVRQNREMFWITLYGRTKELTSELKENFIRFSKSLGLPENHIVFPVPIDQCIDG

SEQ ID NO: 134 - protein, 178 aa, artificial sequence
  Lipocalin mutein
QDSTSDLIPAPPLSKVPLQQNFQDNQFHGKWYVVGIAGNTVLREDKDPGKMPATIYELKEDKSYNVTEVRFAPKKCIYSISTFVPGSQPGEFTLGMIKSSPGGTSALVRVVSTNYNQHAMVFFKKVSQNREVFWITLYGRTKELTSELKENFIRFSKSLGLPENHIVFPVPIDQCIDG

SEQ ID NO: 135 - protein, 178 aa, artificial sequence
  Lipocalin mutein
QDSTSDLIPAPPLSKVPLQQNFQDNQFHGKWYVVGLAGNNILREDKDPAKMPATIYELKEDKSYNVTEVRFSQKKCMYAIYTFVPGSQPGEFTLGRIKSPPGTTSILVRVVSTNYNQHAMVFFKKVMQNREFFWITLYGRTKELTSELKENFIRFSKSLGLPENHIVFPVPIDQCIDG

SEQ ID NO: 136 - protein, 178 aa, artificial sequence
  Lipocalin mutein
QDSTSDLIPAPPLSKVPLQQNFQDNQFHGKWYVVGLAGNHILREDKDPAKMPATIYELKEDKSYNVTEVRFGRKKCHYWIETFVPGSQPGEFTLGRIKSDPGMTSFLVRVVSTNYNQHAMVFFKAVDQNRENFWITLYGRTKELTSELKENFIRFSKSLGLPENHIVFPVPIDQCIDG

SEQ ID NO: 137 - protein, 178 aa, artificial sequence
  Lipocalin mutein
QDSTSDLIPAPPLSKVPLQQNFQDNQFHGKWYVVGNAGNGRLREDKDPPKMWATIYELKEDKSYNVTRVWFNQKKCKYDIETFVPGSQPGEFTLGDIKSTPGWTSNLVRVVSTNYNQHAMVFFKNVMQNREIFYITLYGRTKELTSELKENFIRFSKSLGLPENHIVFPVPIDQCIDG

SEQ ID NO: 138 - protein, 178 aa, artificial sequence
  Lipocalin mutein
QDSTSDLIPAPPLSKVPLQQNFQDNQFHGKWYVVGVAGNTTLREDKDPGKMGATIYELKEDKSYNVTEVRFGRKKCGYWIETFVPGSQPGEFTLGAIKSWPGITSGLVRVVSTNYNQHAMVFFKKVNQNREVFWITLYGRTKELTSELKENFIRFSKSLGLPENHIVFPVPIDQCIDG

SEQ ID NO: 139 - protein, 178 aa, artificial sequence
  Lipocalin mutein
QDSTSDLIPAPPLSKVPLQQNFQDNQFHGKWYVVGVAGNEVLRDDKDPGKMPATIYELKEDKSYNVTEVRFHNKKCNYSIETFVPGSQPGEFTLGKIKSNPGVTSMLVRVVSTNYNQHAMVFFKNVKQNREGFWITLYGRTKELTSELKENFIRFSKSLGLPENHIVFPVPIDQCIDG

SEQ ID NO: 140 - protein, 178 aa, artificial sequence
  Lipocalin mutein
QDSTSDLIPAPPLSKVPLQQNFQDNQFHGKWYVVGVAGNTVLRDDKDPGKMPATIYELKEDKSYNVTEVRFHNKKCNYSIETFVPGSQPGEFTLGNIKSYPGQTSMLVRVVSTNYNQHAMVFFKKVKQNREVFWITLYGRTKELTSELKENFIRFSKSLGLPENHIVFPVPIDQCIDG

SEQ ID NO: 141 - protein, 178 aa, artificial sequence
  Lipocalin mutein
QDSTSDLIPAPPLSKVPLQQNFQDNQFHGKWYVVGVAGNTVLRDDKDPGKMPATIYELKEDKSYNVTEVRFHKKKCNYFIETFVPGSQPGEFTLGKIKSHPGQTSMLVRVVSTNYNQHAMVFFKKVKQNREAFWITLYGRTKELTSELKENFIRFSKSLGLPENHIVFPVPIDQCIDG

SEQ ID NO: 142 - protein, 178 aa, artificial sequence
  Lipocalin mutein
QDSTSDLIPAPPLSKVPLQQNFQDNQFHGKWYVVGVAGNQVLRDDKDPGKMPATIYELKEDKSYNVTEVRFHNKKCNYWIETFVPGSQPGEFTLGNIKSNPGHTSMLVRVVSTNYNQHAMVFFKKVKQNREGFWITLYGRTKELTSELKENFIRFSKSLGLPENHIVFPVPIDQCIDG

SEQ ID NO: 143 - protein, 178 aa, artificial sequence
  Lipocalin mutein
QDSTSDLIPAPPLSKVPLQQNFQDNQFHGKWYVVGVAGNTILREDKDPGKMNATIYELKEDKSYNVTDVRFIFKKCHYYIDTFVPGSQPGEFTLGNIKSYPGMTSQLVRVVSTNYNQHAMVFFKIVRQNREIFWITLYGRTKELTSELKENFIRFSKSLGLPENHIVFPVPIDQCIDG

SEQ ID NO: 144 - protein, 178 aa, artificial sequence
  Lipocalin mutein
QDSTSDLIPAPPLSKVPLQQNFQDNQFHGKWYVVGVAGNTILREDKDPGKMNATIYELKEDKSYNVTDVRFIRKKCHYYIDTFVPGSQPGEFTLGNIKSYPGTTSQLVRVVSTNYNQHAMVFFKIVRQNREIFWITLYGRTKELTSELKENFIRFSKSLGLPENHIVFPVPIDQCIDG

SEQ ID NO: 145 - protein, 178 aa, artificial sequence
  Lipocalin mutein
QDSTSDLIPAPPLSKVPLQQNFQDNQFHGKWYVVGVAGNEVLRDDKDPGKMPATIYELKEDKSYDVTEVRFHNKKCNYFIETFVPGSQPGEFTLGKIKSNPGVTSMLVRVVSTNYNQHAMVFFKNVKQNREGFWITLYGRTKELTSELKENFIRFSKSLGLPENHIVFPVPIDQCIDG

SEQ ID NO: 146 - protein, 178 aa, artificial sequence
  Lipocalin mutein
QDSTSDLIPAPPLSKVPLQQNFQDNQFHGKWYVVGVAGNEVLRDDKDPGKMPATIYELKEDKSYQVTEVRFHNKKCNYFIETFVPGSQPGEFTLGKIKSNPGVTSMLVRVVSTNYNQHAMVFFKNVKQNREGFWITLYGRTKELTSELKENFIRFSKSLGLPENHIVFPVPIDQCIDG

SEQ ID NO: 147 - protein, 178 aa, artificial sequence
  Lipocalin mutein
QDSTSDLIPAPPLSKVPLQQNFQDNQFHGKWYVVGVAGNTILREDKDPGKMNAAIYELKEDKSYDVTDVRFIRKKCHYYIDTFVPGSQPGEFTLGNIKSYPGTTSQLVRVVSTNYNQHAMVFFKIVRQNREIFWITLYGRTKELTSELKENFIRFSKSLGLPENHIVFPVPIDQCIDG

SEQ ID NO: 148 - protein, 178 aa, artificial sequence
  Lipocalin mutein
QDSTSDLIPAPPLSKVPLQQNFQDNQFHGKWYVVGVAGNTILREDKDPGKMNAAIYELKEDKSYQVTDVRFIRKKCHYYIDTFVPGSQPGEFTLGNIKSYPGTTSQLVRVVSTNYNQHAMVFFKIVRQNREIFWITLYGRTKELTSELKENFIRFSKSLGLPENHIVFPVPIDQCIDG

SEQ ID NO: 149 - protein, 178 aa, artificial sequence
  Lipocalin mutein
QDSTSDLIPAPPLSKVPLQQNFQDNQFQGKWYVVGVAGNTILREDKDPGKMNAAIYELKEDKSYDVTDVRFIRKKCHYYIDTFVPGSQPGEFTLGNIKSYPGTTSQLVRVVSTNYNQHAMVFFKIVRQNREIFWITLYGRTKELTSELKENFIRFSKSLGLPENHIVFPVPIDQCIDG

SEQ ID NO: 150 - protein, 178 aa, artificial sequence
  Lipocalin mutein
QDSTSDLIPAPPLSKVPLQQNFQDNQFHGKWYVVGVAGNTILREDKDPGKMNATIYELKEDKSYQVTDVRFIRKKCHYYIDTFVPGSQPGEFTLGNIKSYPGTTSQLVRVVSTNYNQHAMVFFKIVRQNREIFWITLYGRTKELTSELKENFIRFSKSLGLPENHIVFPVPIDQCIDG

SEQ ID NO: 151 - protein, 178 aa, artificial sequence
  Lipocalin mutein
QDSTSDLIPAPPLSKVPLQQNFQDNQFHGKWYVVGFAGNWMLREDKDPHKMNATIYELKEDKSYNVTEVKFQAKKCIYSIHTFVPGSQPGEFTLGIIKSNPGGTSELVRVVSTNYNQHAMVFFKWVHQNREFFQITLYGRTKELTSELKENFIRFSKSLGLPENHIVFPVPIDQCIDG

SEQ ID NO: 152 - protein, 178 aa, artificial sequence
  Lipocalin mutein
QDSTSDLIPAPPLSKVPLQQNFQDNQFHGKWYVVGFAGNRWLREDKDPIKMYATIYELKEDKSYNVTQVNFWLKKCAYSISTFVPGSQPGEFTLGRIKSIPGPTSELVRVVSTNYNQHAMVFFKTVIQNREFFEITLYGRTKELTSELKENFIRFSKSLGLPENHIVFPVPIDQCIDG

SEQ ID NO: 153 - protein, 178 aa, artificial sequence
  Lipocalin mutein
QDSTSDLIPAPPLSKVPLQQNFQDNQFHGKWYVVGFAGNLLLREDKDPRKMRATIYELKEDKSYNVTDVRFLYKKCIYSIATFVPGSQPGEFTLGGIKSAPGFTSELVRVVSTNYNQHAMVFFKWVAQNREYFEITLYGRTKELTSELKENFIRFSKSLGLPENHIVFPVPIDQCIDG

SEQ ID NO: 154 - protein, 178 aa, artificial sequence
  Lipocalin mutein
QDSTSDLIPAPPLSKVPLQQNFQDNQFHGKWYVVGFAGNWRLREDKDPPKMSATIYELKEDKSYNVTNVRFWPKKCRYSISTFVPGSQPGEFTLGMIKSPPGGTSELVRVVSTNYNQHAMVFFKWVFQNREFFEITLYGRTKELTSELKENFIRFSKSLGLPENHIVFPVPIDQCIDG

SEQ ID NO: 155 - protein, 178 aa, artificial sequence
  Lipocalin mutein
QDSTSDLIPAPPLSKVPLQQNFQDNQFHGKWYVVGEAGNLALREDKDPKKMMATIYELKEDKSYNVTEVRFRHKKCQYDIATFVPGSQPGEFTLGLIKSDPGQTSELVRVVSTNYNQHAMVFFKKVVQNREFFWITLYGRTKELTSELKENFIRFSKSLGLPENHIVFPVPIDQCIDG

SEQ ID NO: 156 - protein, 178 aa, artificial sequence
  Lipocalin mutein
QDSTSDLIPAPPLSKVPLQQNFQDNQFHGKWYVVGEAGNLALREDKDPMKMMATIYELKEDKSYNVTEVRFRQKKCKYDIVTFVPGSQPGEFTLGLIKSDPGQTSELVRVVSTNYNQHAMVFFKKVVQNREYFWITLYGRTKELTSELKENFIRFSKSLGLPENHIVFPVPIDQCIDG

SEQ ID NO: 157 - protein, 178 aa, artificial sequence
  Lipocalin mutein
QDSTSDLIPAPPLSKVPLQQNFQDNQFHGKWYVVGEAGNLTLREDKDPMKMMATIYELKEDKSYNVTEVRFRRKKCKYDIVTFVPGSQPGEFTLGVIKSDPGQTSELVRVVSTNYNQHAMVFFKKVVQNREYFWITLYGRTKELTSELKENFIRFSKSLGLPENHIVFPVPIDQCIDG

SEQ ID NO: 158 - protein, 178 aa, artificial sequence
  Lipocalin mutein
QDSTSDLIPAPPLSKVPLQQNFQDNQFHGKWYVVGEAGNLALREDKDPMKMMATIYELKEDKSYNVTEVRFRHKKCKYDIVTFVPGSQPGEFTLGLIKSDPGQTSELVRVVSTNYNQHAMVFFKKVVQNREYFWITLYGRTKELTSELKENFIRFSKSLGLPENHIVFPVPIDQCIDG

SEQ ID NO: 159 - protein, 174 aa, artificial sequence
  Lipocalin mutein
QDSTSDLIPAPPLSKVPLQQNFQDNQFHGKWYVVGEAGNLALREDKDPKKMMATIYELKEDKSYNVTEVRFRYKKCQYDIVTFVPGSQPGEFTLSEPGQTSELVRVVSTNYNQHAMVFFKKVVQNREFFWITLYGRTKELTSELKENFIRFSKSLGLPENHIVFPVPIDQCIDG

SEQ ID NO: 160 - protein, 178 aa, artificial sequence
  Lipocalin mutein
QDSTSDLIPAPPLSKVPLQQNFQDNQFHGKWYVVGEAGNLARREDKDPMKMMATIYELKEDKSYNVTEVRFRHKKCKYDIVTFVPGSQPGEFTLGLIKSDPGQTPELVRVVSTNYNQHAMVFFKKVVQNREYFWITLYGRTKELTSELKENFIRFSKSLGLPENHIVFPVPIDQCIDG

SEQ ID NO: 161 - protein, 178 aa, artificial sequence
  Lipocalin mutein
QDSTSDLIPAPPLSKVPLQQNFQDNQFHGKWYVVGEAGNLALREDKNPMKMMATIYELKEDKSYNVTEVRFRHKKCKYDIVTFVPGSQPGEFTLGLIKSDPGQTPELVRVVSTNYNQHAMVFFKKVVQNREYFWITLYGRTKELPSELKENFIRFSKSLGLPENHIVFPVPIDQCIDG

SEQ ID NO: 162 - protein, 178 aa, artificial sequence
  Lipocalin mutein
QDSTSDLIPAPPLSKVPLQQNFQDNQFHGKWYVVGEAGNLALREGRDPMKMMATIYELKEDKSYNVTEVRFRHKKCKYDIVTFVPGSQPGEFTLGLIKSDPGQTPELVRVVSTNYNQHAMVFFKKVVQNREYFWITLYGRTKELTSELKENFIRFSKSLGLPENHIVFPVPIDQCIDG

SEQ ID NO: 163 - protein, 178 aa, artificial sequence
  Lipocalin mutein
QDSTSDLIPAPPLSKVPLQQNFQDNQFHGKWYVVGEAGNLARREDKDPMKMMATIYELKEDKSYDVTEVRFRHKKCKYDIVTFVPGSQPGEFTLGLIKSDPGQTPELVRVVSTNYNQHAMVFFKKVVQNREYFWITLYGRTKELTSELKENFIRFSKSLGLPENHIVFPVPIDQCIDG

SEQ ID NO: 164 - protein, 178 aa, artificial sequence
  Lipocalin mutein
QDSTSDLIPAPPLSKVPLQQNFQDNQFHGKWYVVGEAGNLALREDKNPMKMMATIYELKEDKSYDVTEVRFRHKKCKYDIVTFVPGSQPGEFTLGLIKSDPGQTPELVRVVSTNYNQHAMVFFKKVVQNREYFWITLYGRTKELPSELKENFIRFSKSLGLPENHIVFPVPIDQCIDG

SEQ ID NO: 165 - protein, 178 aa, artificial sequence
  Lipocalin mutein
QDSTSDLIPAPPLSKVPLQQNFQDNQFHGKWYVVGEAGNLALREGRDPMKMMATIYELKEDKSYDVTEVRFRHKKCKYDIVTFVPGSQPGEFTLGLIKSDPGQTPELVRVVSTNYNQHAMVFFKKVVQNREYFWITLYGRTKELTSELKENFIRFSKSLGLPENHIVFPVPIDQCIDG

SEQ ID NO: 166 - protein, 178 aa, artificial sequence
  Lipocalin mutein
QDSTSDLIPAPPLSKVPLQQNFQDNQFQGKWYVVGEAGNLARREDKDPMKMMATIYELKEDKSYDVTEVRFRHKKCKYDIVTFVPGSQPGEFTLGLIKSDPGQTPELVRVVSTNYNQHAMVFFKKVVQNREYFWITLYGRTKELTSELKENFIRFSKSLGLPENHIVFPVPIDQCIDG

SEQ ID NO: 167 - protein, 178 aa, artificial sequence
  Lipocalin mutein
QDSTSDLIPAPPLSKVPLQQNFQDNQFHGKWYVVGLAGNILLREDKDPHKMLATIYELKEDKSYNVTHVTFKWKKCYYAIRTFVPGSQPGEFTLGMIKSEPGHTSMLVRVVSTNYNQHAMVFFKWVDQNREEFLITLYGRTKELTSELKENFIRFSKSLGLPENHIVFPVPIDQCIDG

SEQ ID NO: 168 - protein, 178 aa, artificial sequence
  Lipocalin mutein
QDSTSDLIPAPPLSKVPLQQNFQDNQFHGKWYVVGHAGNQWLREDKDPRKMWATIYELKEDKSYNVTDVDFAIKKCHYRITTFVPGSQPGEFTLGNIKSHPGGTSGLVRVVSTNYNQHAMVFFKFVIQNREAFFITLYGRTKELTSELKENFIRFSKSLGLPENHIVFPVPIDQCIDG

SEQ ID NO: 169 - protein, 178 aa, artificial sequence
  Lipocalin mutein
QDSTSDLIPAPPLSKVPLQQNFQDNQFHGKWYVVGMAGNFHLREDKDPSKMPATIYELKEDKSYNVTHVPFWAKKCAYKIITFVPGSQPGEFTLGAIKSGPGMTSWLVRVVSTNYNQHAMVFFKGVWQNRETFVITLYGRTKELTSELKENFIRFSKSLGLPENHIVFPVPIDQCIDG

SEQ ID NO: 170 - protein, 178 aa, artificial sequence
  Lipocalin mutein
QDSTSDLIPAPPLSKVPLQQNFQDNQFHGKWYVVGVAGNYWLREDKDPAKMYATIYELKEDKSYNVTDVRFWRKKCRYDIWTFVPGSQPGEFTLGPIKSEPGQTSRLVRVVSTNYNQHAMVFFKLVRQNREAFNITLYGRTKELTSELKENFIRFSKSLGLPENHIVFPVPIDQCIDG

SEQ ID NO: 171 - protein, 178 aa, artificial sequence
  Lipocalin mutein
QDSTSDLIPAPPLSKVPLQQNFQDNQFHGKWYVLGMAGNFHLREDKDPSKMPATIYELKEDKSYNVTHVPFWAKKCAYKTITFVPGSQPGEFTLGAIKSGPGMTSWLVRVVSTNYNQHAMVFSKGVWQNRETFVITLYGRAKELTSELKENFIRFSKSLGLPENHIVFPVPIDQCIDG

SEQ ID NO: 172 - protein, 178 aa, artificial sequence
  Lipocalin mutein
QDSTSDLIPAPPLSKVPLQQNFQDNQFHGKWYVVGMAGNFHLREDKDPSKMPATIYELKEDKSYNVTHVPFWAKKCAYKTITFVPGSQPGEFTLGAIKSGPGMTSWLVRVVSTNYNQHAMVFSKGVWQNRETFVITLYGRTKELTSELKENFIRFSKSLGLPENHIVFPVPIDQCIDG

SEQ ID NO: 173 - protein, 178 aa, artificial sequence
  Lipocalin mutein
QDSTSDLIPAPPLSKVPLQQNFQDNQFHGKWYVVGHAGNQWLREGRDPRKMWATIYELKEDKSYNVTDVDFAIKKCLYRITTFVPGSQPGEFTLGNIKSHPGGTSGLVRVVSTNYNQHAMVFFKFVIQNREAFFITLYGRTKELTSELKENFIRFSKSLGLPENHIVFPVPIDQCIDG

SEQ ID NO: 174 - protein, 178 aa, artificial sequence
  Lipocalin mutein
QDSTSDLIPAPPLSKVPLQQNFQDNQFHGKWYVVGHAGNQWLRGDYDPRKMWATIYELKEDKSYNVTDVDFAIEKCHYRITTFVPGSQPGEFTFGNIKSHPGGTSGLARVVSTNYNQHAMVFFKFVIQNREAFFITLYGRTKELTSELKENFIRFSKSLGLPENHIVFPVPIDQCIDG

SEQ ID NO: 175 - protein, 178 aa, artificial sequence
  Lipocalin mutein
QDSTSDLIPAPPLSKVPLQQNFQDNQFQGKWYVVGHAGNQWLREGRDPRKMWATIYELKEDKSYDVTDVDFAIKKCLYRITTFVPGSQPGEFTLGNIKSHPGGTSGLVRVVSTNYNQHAMVFFKFVIQNREAFFITLYGRTKELTSELKENFIRFSKSLGLPENHIVFPVPIDQCIDG

SEQ ID NO: 176 - protein, 178 aa, artificial sequence
  Lipocalin mutein
QDSTSDLIPAPPLSKVPLQQNFQDNQFHGKWYVVGHAGNQWLRGDYDPRKMWATIYELKEDKSYDVTDVDFAIEKCHYRITTFVPGSQPGEFTFGNIKSHPGGTSGLARVVSTNYNQHAMVFFKFVIQNREAFFITLYGRTKELTSELKENFIRFSKSLGLPENHIVFPVPIDQCIDG

SEQ ID NO: 177 - protein, 152 aa, artificial sequence
  Lipocalin mutein
ASDEEIQDVSGTWYLKAMTVDSRCPRAYYGSVTPMTLTTLEGGNLEAKVTMQRIGRSQEVKAVLEKTDEPGKYTASGGRHVAYIIRSHVKDHYIFYSEGLCPGQPVPGVWLVGRDPKNNLEALEDFEKAAGARGLSTESILIPRQSETSSPG

SEQ ID NO: 178 - protein, 152 aa, artificial sequence
  Lipocalin mutein
ASDEEIQDVSGTWYLKAMTVDSRCPRAYYKSVTPMTLTTLEGGNLEAKFTAQRNGRWQELKLVLEKTDEPGKYAASGGRHVAYIIRSHVKDHYIFYSEGLCPGQPVPGVWLVGRDPKNNLEALEDFEKAAGARGLSTESILIPRQSETSSPG

SEQ ID NO: 179 - protein, 152 aa, artificial sequence
  Lipocalin mutein
ASDEEIQDVSGTWYLKAMTVDSRCPRAYYESVTPMTLTTLEGGNLEAKFTLQRRGRWQEGKLVLEKTDEPGKYTASGGRHVAYIIRSHVKDHYIFYSEGLCPGQPVPGVWLVGRDPKNNLEALEDFEKAAGARGLSTESILIPRQSETSSPG

SEQ ID NO: 180 - protein, 152 aa, artificial sequence
  Lipocalin mutein
ASDEEIQDVSGTWYLKAMTVDSRCPRAYYSSVTPMTLTTLEGGNLEAKFTAQRSGRWQEYKLVLEKTDEPGKYTASGGRHVAYIIRSHVKDHYIFHSEGLCPGQPVPGVWLVGRDPKNNLEALEDFEKAAGARGLSTESILIPRQSETSSPG

SEQ ID NO: 181 - protein, 152 aa, artificial sequence
  Lipocalin mutein
ASDEEIQDVSGTWYLKAMTVDSRCPRAHYSSVTPMTLTTLEGGNLEAKLTLQRAGRWQEGKIVLEKTDEPGKYTASGGRHVAYIIRSHVKDHYIFYSEGLCPGQPVPGVWLVGRDPKNNLEALEDFEKAAGARGLSTESILIPRQSETSSPG

SEQ ID NO: 182 - protein, 152 aa, artificial sequence
  Lipocalin mutein
ASDEEIQDVSGTWYLKAMTVDSRCPRAYYDSVTPMTLTTLEGGNLEAKGTLQRKGRPQEMKLVLEKTDEPGKYTASGGRHVAYIIRSHVKDHYIFYSEGLCPGQPVPGVWLVGRDPKNNLEALEDFEKAAGARGLSTESILIPRQSETSSPG

SEQ ID NO: 183 - protein, 152 aa, artificial sequence
  Lipocalin mutein
ASDEEIQDVSGTWYLKAMTVDSRCPRAYYGSVTPMTLTTLEGGNLEAKLTLQRSGRWQESKVVLEKTDEPGKYTASGGRHVAYIIRSHVKDHYIFYSEGLCPGQPVPGVWLVGRDPKNNLEALEDFEKAAGARGLSTESILIPRQSETSSPG

SEQ ID NO: 184 - protein, 152 aa, artificial sequence; identical to SEQ ID NO: 180
  Lipocalin mutein

SEQ ID NO: 185 - protein, 152 aa, artificial sequence
  Lipocalin mutein
ASDEEIQDVSGTWYLKAMTVDSRCPRAYYESVTPMTLTTLEGGNLEAKLTLQRKGRWQEMKDVLEKTDEPGKYTASGGRHVAYIIRSHVKDHYIFYSEGLCPGQPVPGVWLVGRDPKNNLEALEDFEKAAGARGLSTESILIPRQSETSSPG

SEQ ID NO: 186 - protein, 152 aa, artificial sequence
  Lipocalin mutein
ASDEEIQDVSGTWYLKAMTVDPRCPRAYYSSVTPMTLTTLEGGNLEAKFTAQRSGRWQKYKLVLEKTDEPGKYTASGGRHVAYIIRSHVKDHYIFHSEGLCPGQPVPGVWLVGRDPKNNLEALEDFEKAAGARGLSTESILIPRQSETSSPG

SEQ ID NO: 187 - protein, 152 aa, artificial sequence
  Lipocalin mutein
ASDEEIQDVSGTWYLKAMTVDLRCPRAFYWSVTPMTLTTLEGGNLEAKFTALRIGRWQSYKLVLEKTDEPGKYTASGGRHVAYIIRSHVKDHYIFHSEGLCPGQPVPGVWLVGRDPKNNLEALEDFEKAAGARGLSTESILIPRQSETSSPG

SEQ ID NO: 188 - protein, 152 aa, artificial sequence
  Lipocalin mutein
ASDEEIQDVSGTWYLKAMTVDSRCPRAVYNSVTPMTLTTLEGGNLEAKFTAQRKGRWQKYKLVLEKTDEPGKYTASGGRHVAYIIRSHVKDHYIFHSEGLCPGQPVPGVWLVGRDPKNNLEALEDFEKAAGARGLSTESILIPRQSETSSPG

SEQ ID NO: 189 - protein, 152 aa, artificial sequence
  Lipocalin mutein
ASDEEIQDVSGTWYLKAMTVDSRCPRAYYVSVTPMTLTTLEGGNLEAKFTAARIGRWQSYKLVLEKTDEPGKYTASGGRHVAYIIRSHVKDHYIFHSEGLCPGQPVPGVWLVGRDPKNNLEALEDFEKAAGARGLSTESILIPRQSETSSPG

SEQ ID NO: 190 - protein, 152 aa, artificial sequence
  Lipocalin mutein
ASDEEIQDVSGTWYLKAMTVDNRCPRAKYDSVTPMTLTTLEGGNLEAKFTAHRRGRWQQYKLVLEKTDEPGKYTASGGRHVAYIIRSHVKDHYIFHSEGLCPGQPVPGVWLVGRDPKNNLEALEDFEKAAGARGLSTESILIPRQSETSSPG

SEQ ID NO: 191 - protein, 152 aa, artificial sequence
  Lipocalin mutein
ASDEEIQDVSGTWYLKAMTVDYRCPRAYYHSVTPMTLTTLEGGNLEAKFTAHRAGRWQKYKLVLEKTDEPGKYTASGGRHVAYIIRSHVKDHYIFHSEGLCPGQPVPGVWLVGRDPKNNLEALEDFEKAAGARGLSTESILIPRQSETSSPG

SEQ ID NO: 192 - protein, 152 aa, artificial sequence
  Lipocalin mutein
ASDEEIQDVSGTWYLKAMTVDKRCPRAYYRSVTPMTLTTLEGGNLEAKFTAKRNGRWQPYKLVLEKTDEPGKYTASGGRHVAYIIRSHVKDHYIFHSEGLCPGQPVPGVWLVGRDPKNNLEALEDFEKAAGARGLSTESILIPRQSETSSPG

SEQ ID NO: 193 - protein, 152 aa, artificial sequence
  Lipocalin mutein
ASDEEIQDVSGTWYLKAMTVDERCPRAHYGSVTPMTLTTLEGGNLEAKFTAMRLGRWQKYKLVLEKTDEPGKYTASGGRHVAYIIRSHVKDHYIFHSEGLCPGQPVPGVWLVGRDPKNNLEALEDFEKAAGARGLSTESILIPRQSETSSPG

SEQ ID NO: 194 - protein, 154 aa, artificial sequence
  Lipocalin mutein
ASDEEIQDVSGTWYLKAMTVDSRCPRAVYNSVTPMTLTTLEGGNLEAKFTAQRKGRWQKYKLVLEKTDEPGKYTASGGRHVAYIIRSHVKDHYIFHSEGLCPGQPVPGVWLVGRDPKNNLEALEDFEKAAGARGLSTESILIPRQSETSSPGSD

SEQ ID NO: 195 - protein, 178 aa, artificial sequence
  Lipocalin mutein
QDSTSDLIPAPPLSKVPLQQNFQDNQFHGKWYVVGVAGNYTLREDKDPLKMYATIYELKEDKSYNVTSVGFRLKKCNYKIRTFVPGSQPGEFTLGIIKSQPGMTSYLVRVVSTNYNQHAMVFFKTVGQNREMFNITLYGRTKELTSELKENFIRFSKSLGLPENHIVFPVPIDQCIDG

SEQ ID NO: 196 - protein, 178 aa, artificial sequence
  Lipocalin mutein
QDSTSDLIPAPPLSKVPLQQNFQDNQFHGKWYVVGVAGNKSLREDKDPWKMYATIYELKEDKSYNVTSVGFGTKKCHYKIRTFVPGSQPGEFTLGRIKSRPGRTSALVRVVSTNYNQHAMVFFKVVQQNRESFNITLYGRTKELTSELKENFIRFSKSLGLPENHIVFPVPIDQCIDG

SEQ ID NO: 197 - protein, 178 aa, artificial sequence
  Lipocalin mutein
QDSTSDLIPAPPLSKVPLQQNFQDNQFHGKWYVVGCAGNVLLREDKDPLKMYATIYELKEDKSYNVTSVGFDDKKCLYKIRTFVPGSQPGEFTLGRIKSEPGGTSWLVRVVSTNYNQHAMVFFKEVAQNRETFNITLYGRTKELTSELKENFIRFSKSLGLPENHIVFPVPIDQCIDG

SEQ ID NO: 198 - protein, 178 aa, artificial sequence
  Lipocalin mutein
QDSTSDLIPAPPLSKVPLQQNFQDNQFHGKWYVVGAAGNVLLREDKDPLKMYATIYELKEDKSYNVTSVGFDDKKCLYKIRTFVPGSQPGEFTLGRIKSEPGGTSWLVRVVSTNYNQHAMVFFKEVAQNRETFNITLYGRTKELTSELKENFIRFSKSLGLPENHIVFPVPIDQCIDG

SEQ ID NO: 199 - protein, 178 aa, artificial sequence
  Lipocalin mutein
QDSTSDLIPAPPLSKVPLQQNFQDNQFHGKWYVVGVAGNVLLREDKDPLKMYATIYELKEDKSYNVTSVGFDDKKCLYKIRTFVPGSQPGEFTLGRIKSEPGGTSWLVRVVSTNYNQHAMVFFKEVAQNRETFNITLYGRTKELTSELKENFIRFSKSLGLPENHIVFPVPIDQCIDG

SEQ ID NO: 200 - protein, 178 aa, artificial sequence
  Lipocalin mutein
QDSTSDLIPAPPLSKVPLQQNFQDNQFHGKWYVVGKAGNHDLREDKDPRKMQATIYELKEDKSYNVTNVRFVHKKCNYRIWTFVPGSQPGEFTLGNIKSWPGLTSWLVRVVSTNYNQHAMVFFKRVYQNRELFEITLYGRTKELTNELKENFIRFSKSLGLPENHIVFPVPIDQCIDG

SEQ ID NO: 201 - protein, 178 aa, artificial sequence
  Lipocalin mutein
QDSTSDLIPAPPLSKVPLQQNFQDNQFHGKWYVVGEAGNSLLREDKDPRKMYATIYELKEDKSYNVTSVRFRSKKCHYLIRTFVPGSQPGEFTLGLIKSKPGHTSFLVRVVSTNYNQHAMVFFKTVAQNREYFFITLYGRTKELTSELKENFIRFSKSLGLPENHIVFPVPIDQCIDG

SEQ ID NO: 202 - protein, 178 aa, artificial sequence
  Lipocalin mutein
QDSTSDLIPAPPLSKVPLQQNFQDNQFHGKWYVVGRAGNMRLREDKDPAKMVATIYELKEDKSYNVTKVMFQRKKCKYMINTFVPGSQPGEFTLGAIKSPPGPTSTLVRVVSTNYNQHAMVFFKHVFQNREYFHITLYGRTKELTSELKENFIRFSKSLGLPENHIVFPVPIDQCIDG

SEQ ID NO: 203 - protein, 178 aa, artificial sequence
  Lipocalin mutein
QDSTSDLIPAPPLSKVPLQQNFQDNQFHGKWYVVGAAGNTWLREDKDPYKMQATIYELKEDKSYNVTNVLFMSKKCRYMIHTFVPGSQPGEFTLGSIKSWPGLTSWLVRVVSTNYNQHAMVFFKRVYQNREFFGITLYGRTKELTSELKENFIRFSKSLGLPENHIVFPVPIDQCIDG

SEQ ID NO: 204 - protein, 445 aa, artificial sequence
  Antibody heavy chain
QVQLVQSGAEVKKPGSSVKVSCKASGYTFGNYWMQWVRQAPGQGLEWMGAIYEGTGKTVYIQKFADRVTITADKSTSTAYMELSSLRSEDTAVYYCARLSDYVSGFGYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 205 - protein, 214 aa, Artificial sequence
  Antibody light chain
DIQMTQSPSSLSASVGDRVTITCRASKDISKYLNWYQQKPGKAPKLLIYYTSGYHSGVPSRFSGSGSGTDFTLTISSLQPEDFATYYCQQGDALPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 206 - protein, 178 aa, artificial sequence; identical to SEQ ID NO: 38
  Lipocalin mutein

SEQ ID NO: 207 - protein, 178 aa, artificial sequence; identical to SEQ ID NO: 21
  Lipocalin mutein

SEQ ID NO: 208 - protein, 178 aa, artificial sequence
  Lipocalin mutein
QDSTSDLIPAPPLSKVPLQQNFQDNQFHGKWYVVGRAGNDVLREDKDPEKMEATIYELKEDKSYQVTDVGFVQKRCMYVTHTFVPGSQPGEFTLGNIESYPGVTSALVRVVSTDYNQHAMVFVKLVKQNRELFEITLYGRTKELTSELKENFIRFSKSLGLPENHIVFPVPIDQCIDD

SEQ ID NO: 209 - protein, 178 aa, artificial sequence
  Lipocalin mutein
QDSTSDLKPAPPLSKVPLQQNFQDNQFHGKWYVVGRAGNDVLREDKDPEKMEATIYELKEDKSYNVTDVGFVQKRCMYVTHTFVPGSQPGEFTLGNIESYPGVTSALVRVVSTDYNQHAMVFVKLVKQNRELFEITLYGRTKELTSELKENFIRFSKSLGLPENHIVFPVPIDQCIDG

SEQ ID NO: 210 - protein, 178 aa, artificial sequence
  Lipocalin mutein
QDSTSDLIHAPPLSKVPLQQNFQDNQFHGKWYVVGRAGNDVLREDKDPEKMEATIYELKEDKSYNVTDVGFVQKRCMYVTHTFVPGSQPGEFTLGNIESYPGVTSALVRVVSTDYNQHAMVFVKLVKQNRELFEITLYGRTKELTSELKENFIRFSKSLGLPENHIVFPVPIDQCIDG

SEQ ID NO: 211 - protein, 178 aa, artificial sequence
  Lipocalin mutein
QDSTSDLKPAPPLSKVPLQQNFQDNQFHGKWYVVGRAGNDVLREDKDPEKMEATIYELKEDKSYQVTDVGFVQKRCMYVTHTFVPGSQPGEFTLGNIESYPGVTSALVRVVSTDYNQHAMVFVKLVKQNRELFEITLYGRTKELTSELKENFIRFSKSLGLPENHIVFPVPIDQCIDD

SEQ ID NO: 212 - protein, 178 aa, artificial sequence
  Lipocalin mutein
QDSTSDLIHAPPLSKVPLQQNFQDNQFHGKWYVVGRAGNDVLREDKDPEKMEATIYELKEDKSYQVTDVGFVQKRCMYVTHTFVPGSQPGEFTLGNIESYPGVTSALVRVVSTDYNQHAMVFVKLVKQNRELFEITLYGRTKELTSELKENFIRFSKSLGLPENHIVFPVPIDQCIDD

(19) United States
(12) Patent Application Publication
(10) Pub. No.: US 2022/0168387 A1
(43) Pub. Date: Jun. 2, 2022

(54) INHALED ADMINISTRATION OF LIPOCALIN MUTEINS

(71) Applicant: PIERIS PHARMACEUTICALS GMBH, Hallbergmoos (DE)

(72) Inventors: Gabriele MATSCHINER, Munich (DE); Ahmed MOUSA, Cambridge, MA (US); Vanessa WELK, Munich (DE); Gary ANDERSON, Parkville (AU); Christine WRIGHT, Parkville (AU); Thomas JAQUIN, Erding (DE)

(73) Assignee: PIERIS PHARMACEUTICALS GMBH, Hallbergmoos (DE)

(21) Appl. No.: 17/599,765

(22) PCT Filed: Mar. 27, 2020
(86) PCT No.: PCT/EP2020/058637

§ 371 Date: Sep. 29, 2021

Related U.S. Application Data
(60) Provisional application No. 62/826,767, filed on Mar. 29, 2019.

(30) Foreign Application Priority Data
Apr. 1, 2019 (EP) 19166435.8
May 22, 2019 (EP) 19175818.4
May 31, 2019 (EP) 19177568.3
Nov. 26, 2019 (EP) 19211404.9

(51) Int. Cl. A61K 38/17; A61K 9/00

(52) U.S. Cl. CPC A61K 38/1709; A61K 9/0073

(57) ABSTRACT

The present invention relates to inhaled administration of a lipocalin mutein to a subject, wherein the administration provides for local exposure to the lipocalin mutein in the respiratory tract. The present invention also relates to inhalative administration of a lipocalin mutein to a subject, wherein the administration provides for systemic exposure to the lipocalin mutein.

### I. BACKGROUND

Drug delivery in the airways by inhalation can be used for local and/or systemic action, depending on the therapeutic need and ability of the aerosolized drug to cross the air blood barrier. Inhaled drugs are delivered to the lungs, where the good vascularization, immense capacity for solute exchange, and ultra-thinness of the alveolar epithelium are unique features that can facilitate systemic delivery via pulmonary administration of peptides and proteins (Agu et al., Respir Res, 2001). However, a number of molecule- and administration-route-related challenges remain in the art.

Lipocalins are proteins scaffolds able to accommodate a great variety of targets, in terms of size, shape and chemical character (Skerra, Biochim Biophys Acta, 2000). Lipocalins share a highly conserved overall folding structure composed of a four-loop variable region mounted on a stable β-barrel scaffold (Skerra, FEBS J, 2008). Recently, members of the lipocalin family have become subject of research as target-binding proteins, a crucial role in life sciences in general, which has been mostly occupied by antibodies (immunoglobulines) (WO 99/16873, WO03/029463, WO 03/029471, Schlehuber and Skerra, Biophys Chem, 2002, Skerra, J Biotechnol, 2001). Lipocalin muteins are a class of molecules based on the lipocalin structure and generated via mutagenesis of their binding site to further increases their plasticity, thus allowing such muteins to bind to selected targets.

Currently, there is no approved system for inhaled delivery of antibodies or approved inhaled antibody therapeutic. While there are a number of approved small molecules for inhalation, they carry a number of drawbacks, including low targeting affinity, off-target binding, and side effects on other organs. Inhaled biological therapeutics, including proteins (e.g., antibodies and antibody-like molecules) and peptides, may serve as alternatives, providing increased targeting-binding ability and potency as well as reduced off-target effects. However, inhaled administration of proteins and peptides imposes stringent requirements on the delivery device, and certain barriers, particularly the respiratory epithelium, compromise the absorption and total and regional (e.g., distal lung) deposition of the inhaled proteins and peptides. It thus remains a need in the art to provide efficient delivery of protein-based therapies such as antibodies or antibody-like therapeutics by inhalation. The technical problem underlying the present application is to comply with said need. The technical problem is solved by providing the embodiments reflected in the claims, described in the description and illustrated in the examples and figures that follow.

### II. DEFINITIONS

The following list defines terms, phrases, and abbreviations used throughout the instant specification. All terms listed and defined herein are intended to encompass all grammatical forms.

As used herein, “detectable affinity” means the ability to bind to a selected target with an affinity, generally measured by K_{d} or EC₅₀, of at most about 10⁻⁵ M or below (a lower K_{d} or EC₅₀ value reflects better binding activity). Lower affinities are generally no longer measurable with common methods such as ELISA (enzyme-linked immunosorbent assay) and therefore of secondary importance.

As used herein, “binding affinity” of a protein of the disclosure (e.g. a mutein of a lipocalin) or a fusion polypeptide thereof to a selected target, can be measured (and thereby K_{d} values of a mutein-ligand complex can be determined) by a multitude of methods known to those skilled in the art. Such methods include, but are not limited to, fluorescence titration, competitive ELISA, calorimetric methods, such as isothermal titration calorimetry (ITC), and surface plasmon resonance (SPR). Such methods are well established in the art and examples thereof are also detailed below.

It is also noted that the complex formation between the respective binder and its ligand is influenced by many different factors such as the concentrations of the respective binding partners, the presence of competitors, pH and the ionic strength of the buffer system used, and the experimental method used for determination of the dissociation constant K_{d} (for example fluorescence titration, competition ELISA or surface plasmon resonance, just to name a few) or even the mathematical algorithm which is used for evaluation of the experimental data.

Therefore, it is also clear to the skilled person that the K_{d} values (dissociation constant of the complex formed between the respective binder and its target/ligand) may vary within a certain experimental range, depending on the method and experimental setup that is used for determining the affinity of a particular lipocalin mutein for a given ligand. This means that there may be a slight deviation in the measured K_{d} values or a tolerance range depending, for example, on whether the K_{d} value was determined by surface plasmon resonance (SPR), by competitive ELISA, or by direct ELISA.

As used herein, a “mutein,” a “mutated” entity (whether protein or nucleic acid), or “mutant” refers to the exchange, deletion, or insertion of one or more nucleotides or amino acids, compared to the naturally-occurring (wild-type) nucleic acid or protein “reference” scaffold. The “reference scaffold” is preferably mature human tear lipocalin or mature human neutrophil gelatinase-associated lipocalin. Said “reference scaffold” also includes fragments of a mutein and variants as described herein.

As used herein, “tear lipocalin” refers to human tear lipocalin (hTlc) and further refers to mature human tear lipocalin. The term “mature” when used to characterize a protein means a protein essentially free from the signal peptide. A “mature hTlc” of the disclosure refers to the mature form of human tear lipocalin, which is free from the signal peptide. Mature hTlc is described by residues 19-176 of the sequence deposited with the SWISS-PROT Data Bank under Accession Number P31025, and the amino acid of which is indicated in SEQ ID NO: 1.

As used herein, “Lipocalin-2” or “neutrophil gelatinase-associated lipocalin” refers to human Lipocalin-2 (hLcn2) or human neutrophil gelatinase-associated lipocalin (hNGAL) and further refers to the mature hLcn2 or mature hNGAL. The term “mature” when used to characterize a protein means a protein essentially free from the signal peptide. A “mature hNGAL” of the instant disclosure refers to the mature form of human neutrophil gelatinase-associated lipocalin, which is free from the signal peptide. Mature hNGAL is described by residues 21-198 of the sequence deposited with the SWISS-PROT Data Bank under Accession Number P80188, and the amino acid of which is indicated in SEQ ID NO: 2.

The term “fragment” as used herein in connection with the muteins of the disclosure relates to proteins or peptides derived from said mutein, such as a full-length mature human tear lipocalin (hTlc or hTLPC) or a full-length mature human neutrophil gelatinase-associated lipocalin (hNGAL), that is N-terminally and/or C-terminally truncated, i.e. lacking at least one of the N-terminal and/or C-terminal amino acids. Such a fragment may lack up to 2, up to 3, up to 4, up to 5, up to 10, up to 15, up to 20, up to 25, or up to 30 (including all numbers in between) of the N-terminal and/or C-terminal amino acids. As an illustrative example, such a fragment may lack the one, two, three, or four N-terminal and/or one or two C-terminal amino acids, especially if the mutein is derived from hTlc. It is understood that the fragment is preferably a functional fragment of a full-length lipocalin (mutein), which means that it preferably comprises the binding pocket of the full length lipocalin (mutein) it is derived from. As an illustrative example, such a functional fragment may comprise at least amino acids at positions 5-158, 1-156, 5-156, 5-153, 5-150, 9-148, 12-140, 20-135, or 26-133 corresponding to the linear polypeptide sequence of mature hTlc. As another illustrative example, such a functional fragment may comprise at least amino acids at positions 5-168, 8-160, 13-157, 15-150, 18-141, 20-134, 25-134, or 28-134 corresponding to the linear polypeptide sequence of mature hNGAL. Such fragments may include at least 10, more such as 20 or 30 or more consecutive amino acids of the primary sequence of the mature lipocalin and are usually detectable in an immunoassay of the mature lipocalin. A fragment may have at least about 50%, 60%, 70%, 75%, 80%, 85%, 90%, 92%, 95% or at least about 98% amino acid sequence identity with the native sequence of the protein or polypeptide. In general, the term “fragment,” as used herein with respect to the corresponding protein target of a lipocalin mutein of the disclosure, relates to N-terminally and/or C-terminally shortened protein or peptide ligands, which retain the capability of the full length ligand to be recognized and/or bound by a mutein according to the disclosure.

As used herein, the term “variant” relates to derivatives of a protein or polypeptide that include mutations, for example by substitutions, deletions, insertions, and/or chemical modifications of an amino acid sequence or nucleotide sequence. In some embodiments, such mutations and/or chemical modifications do not reduce the functionality of the protein or peptide. Such substitutions may be conservative, i.e., an amino acid residue is replaced with a chemically similar amino acid residue. Examples of conservative substitutions are the replacements among the members of the following groups: 1) alanine, serine, threonine, and valine; 2) aspartic acid, glutamic acid, glutamine, and asparagine, and histidine; 3) arginine, lysine, glutamine, asparagine, and histidine; 4) isoleucine, leucine, methionine, valine, alanine, phenylalanine, threonine, and proline; and 5) isoleucine, leucine, methionine, phenylalanine, tyrosine, and tryptophan. Such variants include proteins or polypeptides, wherein one or more amino acids have been substituted by their respective D-stereoisomers or by amino acids other than the naturally occurring 20 amino acids, such as, for example, ornithine, hydroxyproline, citrulline, homoserine, hydroxylysine, norvaline. Such variants also include, for instance, proteins or polypeptides in which one or more amino acid residues are added or deleted at the N- and/or C-terminus. Generally, a variant has at least about 50%, 60%, 70%, 75%, 80%, 85%, 90%, 92%, 95% or at least about 98% amino acid sequence identity with the native sequence protein or polypeptide. A variant preferably retains the biological activity, e.g. binding the same target, of the protein or polypeptide it is derived.

The term “mutagenesis” as used herein means that the experimental conditions are chosen such that the amino acid naturally occurring at a given sequence position of the mature lipocalin can be substituted by at least one amino acid that is not present at this specific position in the respective natural polypeptide sequence. The term “mutagenesis” also includes the (additional) modification of the length of sequence segments by deletion or insertion of one or more amino acids. Thus, it is within the scope of the disclosure that, for example, one amino acid at a chosen sequence position is replaced by a stretch of three random mutations, leading to an insertion of two amino acid residues compared to the length of the respective segment of the wild-type protein. Such an insertion or deletion may be introduced independently from each other in any of the peptide segments that can be subjected to mutagenesis in the disclosure. In one exemplary embodiment of the disclosure, an insertion of several mutations may be introduced into the loop AB of the chosen lipocalin scaffold (cf. International Patent Publication No. WO 2005/019256 which is incorporated by reference its entirety herein).

As used herein, the term “random mutagenesis” means that no predetermined mutation (alteration of amino acid) is present at a certain sequence position but that at least two amino acids can be incorporated with a certain probability at a predefined sequence position during mutagenesis.

As used herein, the term “sequence identity” or “identity” denotes a property of sequences that measures their similarity or relationship. The term “sequence identity” or “identity” as used in the present disclosure means the percentage of pair-wise identical residues following (homologous) alignment of a sequence of a protein or polypeptide of the disclosure with a sequence in question with respect to the number of residues in the longer of these two sequences. Sequence identity is measured by dividing the number of identical amino acid residues by the total number of residues and multiplying the product by 100.

As used herein, the term “sequence homology” or “homology” has its usual meaning and homologous amino acid includes identical amino acids as well as amino acids which are regarded to be conservative substitutions at equivalent positions in the linear amino acid sequence of a protein or polypeptide of the disclosure (e.g., any fusion proteins or lipocalin muteins of the disclosure).

A skilled artisan will recognize available computer programs, for example BLAST (Altschul et al., Nucleic Acids Res, 1997), BLAST2 (Altschul et al., J Mol Biol, 1990), TBLASTN (Altschul et al., J Mol Biol, 1990), FASTA (Pearson and Lipman, Proc Natl Acad Sci USA, 1988), Gap (Wisconsin GCG package, Accelerys Inc), and Smith-Waterman (Smith and Waterman, J Mol Biol, 1981), for determining sequence homology or sequence identity using standard parameters. The percentage of sequence homology or sequence identity can, for example, be determined herein using the program BLASTP, version 2.2.5, Nov. 16, 2002 (Altschul et al., Nucleic Acids Res, 1997). In this embodiment, the percentage of homology is based on the alignment of the entire protein or polypeptide sequences (matrix: BLOSUM 62; gap costs: 11.1; cutoff value set to 10⁻³) including the propeptide sequences, preferably using the wild-type protein scaffold as reference in a pairwise comparison. It is calculated as the percentage of numbers of “positives” (homologous amino acids) indicated as result in the BLASTP program output divided by the total number of amino acids selected by the program for the alignment.

Specifically, in order to determine whether an amino acid residue of the amino acid sequence of a lipocalin (mutein) is different from a wild-type lipocalin corresponding to a certain position in the amino acid sequence of a wild-type lipocalin, a skilled artisan can use means and methods well-known in the art, e.g., alignments, either manually or by using computer programs such as BLAST 2.0, which stands for Basic Local Alignment Search Tool, or ClustalW, or any other suitable program which is suitable to generate sequence alignments. Accordingly, a wild-type sequence of lipocalin can serve as “subject sequence” or “reference sequence”, while the amino acid sequence of a lipocalin different from the wild-type lipocalin described herein serves as “query sequence”. The terms “wild-type sequence” and “reference sequence” and “subject sequence” are used interchangeably herein. A preferred wild-type sequence of human tear lipocalin is the sequence of mature human tear lipocalin as shown in SEQ ID NO: 1. A preferred wild-type sequence of hNGAL is the sequence of mature hNGAL as shown in SEQ ID NO: 2.

“Gaps” are spaces in an alignment that are the result of additions or deletions of amino acids. Thus, two copies of exactly the same sequence have 100% identity, but sequences that are less highly conserved, and have deletions, additions, or replacements, may have a lower degree of sequence identity. Those skilled in the art will recognize that several computer programs are available for determining sequence identity using standard parameters, for example BLAST (Altschul et al., Nucleic Acids Res, 1997), BLAST2 (Altschul et al., J Mol Biol, 1990), TBLASTN (Altschul et al., J Mol Biol, 1990), FASTA (Pearson and Lipman, Proc Natl Acad Sci USA, 1988), Gap (Wisconsin GCG package, Accelerys Inc), and Smith-Waterman (Smith and Waterman, J Mol Biol, 1981).

As used herein, the term “position” means the position of either an amino acid within an amino acid sequence depicted herein or the position of a nucleotide within a nucleic acid sequence depicted herein. It is to be understood that when the term “correspond” or “corresponding” as used herein in the context of the amino acid sequence positions of one or more lipocalin muteins, a corresponding position is not only determined by the number of the preceding nucleotides or amino acids. Accordingly, the absolute position of a given amino acid in accordance with the disclosure may vary from the corresponding position due to deletion or addition of amino acids elsewhere in a (mutant or wild-type) lipocalin. Similarly, the absolute position of a given nucleotide in accordance with the present disclosure may vary from the corresponding position due to deletions or additional nucleotides elsewhere in a mutein or wild-type lipocalin 5′-untranslated region (UTR) including the promoter and/or any other regulatory sequences or gene (including exons and introns).

A “corresponding position” in accordance with the disclosure may be the sequence position that aligns to the sequence position it corresponds to in a pairwise or multiple sequence alignment according to the present disclosure. It is preferably to be understood that for a “corresponding position” in accordance with the disclosure, the absolute positions of nucleotides or amino acids may differ from adjacent nucleotides or amino acids but said adjacent nucleotides or amino acids which may have been exchanged, deleted, or added may be comprised by the same one or more “corresponding positions”.

In addition, for a corresponding position in a lipocalin mutein based on a reference sequence in accordance with the disclosure, it is preferably to be understood that the positions of nucleotides or amino acids of a lipocalin mutein can structurally correspond to the positions elsewhere in a reference lipocalin (wild-type lipocalin) or another lipocalin mutein, even if they may differ in the absolute position numbers, as appreciated by the skilled in light of the highly-conserved overall folding pattern among lipocalins.

As used herein, “antibody” includes whole antibodies or any antigen binding fragment (i.e., “antigen-binding portion”) or single chain thereof. A whole antibody refers to a glycoprotein comprising at least two heavy chains (HCs) and two light chains (LCs) inter-connected by disulfide bonds. Each heavy chain is comprised of a heavy chain variable domain (V_{H} or HCVR) and a heavy chain constant region (C_{H}). The heavy chain constant region is comprised of three domains, C_{H1}, C_{H2} and C_{H3}. Each light chain is comprised of a light chain variable domain (V_{L} or LCVR) and a light chain constant region (C_{L}). The light chain constant region is comprised of one domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} is composed of three CDRs and four FRs, arranged in the following order from the amino-terminus to the carboxy-terminus: FR1, CDR1, FR2, CDR2, FR3, CDR3, FR4. The variable regions of the heavy and light chains contain a binding domain that interacts with an antigen. The constant regions of the antibodies may optionally mediate the binding of the immunoglobulin to host tissues or factors, including various cells of the immune system (e.g., effector cells) and the first component (C1q) of the classical complement system.

As used herein, “antigen binding fragment” of an antibody refers to one or more fragments of an antibody that retain the ability to specifically bind to an antigen (e.g., GPC3). It has been shown that the antigen-binding function of an antibody can be performed by fragments of a full-length antibody. Examples of binding fragments encompassed within the term “antigen-binding fragment” of an antibody include (i) a Fab fragment consisting of the V_{H}, V_{L}, C_{L} and C_{H1} domains; (ii) a F(ab′)₂ fragment comprising two Fab fragments linked by a disulfide bridge at the hinge region; (iii) a Fab′ fragment consisting of the V_{H}, V_{L}, C_{L} and C_{H1} domains and the region between C_{H1} and C_{H2} domains; (iv) a Fd fragment consisting of the V_{H} and C_{H1} domains; (v) a single-chain Fv fragment consisting of the V_{H} and V_{L} domains of a single arm of an antibody, (vi) a dAb fragment (Ward et al., Nature, 1989) consisting of a V_{H} domain; and (vii) an isolated complementarity determining region (CDR) or a combination of two or more isolated CDRs which may optionally be joined by a synthetic linker; (viii) a “diabody” comprising the V_{H} and V_{L} connected in the same polypeptide chain using a short linker (see, e.g., patent documents EP 404,097; WO 93/11161; and Holliger et al., Proc Natl Acad Sci USA, 1993); (ix) a “domain antibody fragment” containing only the V_{H} or V_{L}, where in some instances two or more V_{H} regions are covalently joined.

Antibodies may be polyclonal or monoclonal; xenogeneic, allogeneic, or syngeneic; or modified forms thereof (e.g., humanized, chimeric, or multispecific). Antibodies may also be fully human.

A “subject” is a vertebrate, preferably a mammal, more preferably a human. The term “mammal” is used herein to refer to any animal classified as a mammal, including, without limitation, humans, domestic and farm animals, and zoo, sports, or pet animals, such as sheep, dogs, horses, cats, cows, rats, pigs, apes such as cynomolgus monkeys, and etc., to name only a few illustrative examples. Preferably, the “mammal” herein is human, mouse, or a non-human primate. Preferably, the subject is human.

An “effective amount” is an amount sufficient to effect beneficial or desired results. An effective amount can be administered in one or more administrations.

A “sample” is defined as a biological sample taken from any subject. Biological samples include, but are not limited to, blood, serum, urine, feces, semen, or tissue.

As used herein “inhaled administration” or “administration by inhalation” refers to administration of a substance via the respiratory tract, usually by oral inhalation or nasal inhalation. The substance may be in the form of a gas, a liquid aerosol, a fine powder, or a liquid spray. Inhaled administration may be carried out using an inhaler.

An “administered dose” corresponds to the dose of a compound that has been administered to a subject. In the context of intratracheal administration of a substance using a microsprayer device, the “administered dose” corresponds to the “delivered dose”.

A “metered dose” or “device dose”, in particular in the context of an inhalation device, relates to the dose of a substance a device has been loaded with.

A “delivered dose” refers to the dose of a substance that is delivered to a subject, i.e. the dose that comes out of an inhalation device when applying the device. For example, nebulizers are sometimes intentionally overfilled as the final total volume will not be nebulised. For a nebulizer, a delivered dose is commonly less than 50% of the nominal dose, which is the total active substance loaded into the device. The nominal dose is also known as the device dose or metered dose. For a dry powder inhaler, the delivered dose is commonly about 85-90% of the metered dose. A skilled person can easily determine a delivered dose by determining the amount of a substance that comes out of the inhalation device. For example, methods used to measure the “delivered dose” experimentally are provided in section 2.9.44 of the European Pharmacopeia 9.0.

As used herein “local exposure” or “local administration” means that no substantive portion of a locally-administered substance enters the circulatory system. Preferably, the amount of the substance that enters the circulatory system is below the limit of quantification (BLQ). In other instances, the amount of the substance that enters the circulatory system can be measured but would not be considered substantive. In the context of inhaled administration of a substance, “local exposure” or “local administration” may mean that the substance essentially remains in the respiratory system. Since in some cases, in particular if the subject is human, direct measurement of the amount of a substance that remains in the respiratory system is difficult to measure, determination of “local exposure” or “local administration” is preferably carried out indirectly by determining the amount of the substance that enters the circulatory system.

As used herein “systemic exposure” means that a substantive portion of the locally-administered substance enters the circulatory system and, optionally, that the entire body may be affected by the substance. Systemic exposure may mean that the amount of the substance that enters the circulatory system in quantifiable. Systemic exposure may equate to the concentration of substance that enters the bloodstream that is quantifiable. This exposure can be represented by the blood (serum, plasma or whole blood) concentration of the substance which can be measured over time and recorded by a range of parameters including the area under the curve (AUC). Systemic exposure to substance can also impact biomarkers, the levels of which can correlate directly to concentration of substance and therefore to systemic exposure. The term “quantifiable” or “detectable,” when used in connection with systemic exposure, refers to the exposure represented by the blood (serum, plasma or whole blood) concentration of the substance or by the levels of biomarkers measurable by one or more analytical methods known in art. Such analytical methods include, but are not limited to, ELISA, competitive ELISA, fluorescence titration, calorimetric methods, mass spectrometry (MS), and chromatography methods, such as high-performance liquid chromatography (HPLC). It is also understood measurements performed using such analytical methods are associated with detection limits, such as instrument detection limit, method detection limits, and limit of quantification.

As used herein “onset” or “onset of action” of a drug refers to the duration of time it takes for a drug's effect to come to prominence upon administration. In some embodiments, the drug's effect may be considered prominent upon reaching, e.g., 50%, 60%, 70%, 80%, 90%, or 100% of the maximum therapeutic effect. In some embodiments, the drug's effect may be considered prominent when the symptom(s) of the subject to which the drug is administered is relieved. The onset of a drug may be quantified by determining the time from the end of any administration of such a drug to reaching a desired level, e.g., 90% or maximal level, of change in the therapeutic effect of the drug compared to baseline. In some particular embodiments, the onset of drug may be determined, as described in Example 4, as the duration of time to achieve 50%, 60%, 70%, 80%, 90%, or ever higher percentage reduction of carotid vascular resistance as compared to baseline. The onset of a drug may e.g. be about 1 to 5 minutes, about 1 to 25 minutes, about 5 to 25 minutes, or about 10 to 20 minutes.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. The term, however, also includes the concrete number, e.g., “about 20” includes 20.

### III. DESCRIPTIONS OF FIGURES

FIG. 1: provides the result of pharmacokinetic analyses in mice of SEQ ID NO: 3 (lipocalin mutein of hNGAL, FIG. 1A) and SEQ ID NO: 4 (lipocalin mutein of hTlc, FIG. 1B), as described in Example 1. Mice were intratracheally administered with test lipocalin muteins at a dose of 100 μg/kg. Drug levels in bronchoalveolar lavage fluid (BALF), normalized lung homogenates, and blood plasma were detected using an electrochemiluminescence (ECL)-based assay. The lipocalin muteins display different concentrations with similar PK profiles in each of the three compartments.

FIG. 2: provides the result of pharmacokinetic analyses in mice intratracheally administered with SEQ ID NO: 3 (lipocalin mutein of hNGAL, FIG. 2A) or SEQ ID NO: 4 (lipocalin mutein of hTlc, FIG. 2B) at a dose of 100 μg/mouse, as described in Example 2. Drug levels in BALF, normalized lung homogenates, and blood plasma were detected using an ECL-based assay. The lipocalin muteins display similar PK profiles in each of the three compartments. Both lipocalin muteins show time-dependent decrease in concentrations in all three compartments, but with higher exposure levels as measured by the AUC_{inf} in BALF than lung, which is greater than plasma.

FIG. 3: provides the result of pharmacokinetic analyses in mice injected intravenously with SEQ ID NO: 3 (lipocalin mutein of hNGAL) or SEQ ID NO: 4 (lipocalin mutein of hTlc) at a dose of 2 mg/kg, as described in Example 3. Serum drug levels were detected using an ECL-based assay. The two lipocalin muteins display similar PK profiles.

FIG. 4: provides the results of sensory nerve-mediated vasodilatation in rat treated with an exemplary lipocalin mutein (SEQ ID NO: 47) after intravenous administration at a dose of 1 mg/kg, subcutaneous administration at a dose of 5 mg/kg, intratracheal administration at a dose of 5 mg/kg, or intratracheal administration with fumaryl diketopiperazine (FDKP) at a dose of 5 mg/kg. As a control, a reference anti-CGRP antibody (SEQ ID NOs: 204 and 205) was also tested via intravenous administration, as described in Example 4. In lipocalin mutein-treated animals, the dermal blood flow in the dorsomedial skin of the rat hind paw after nerve stimulation is significantly decreased from that seen in untreated animals, with a maximal change comparable to that observed of the reference anti-CGRP antibody, indicating increased vasoconstriction through blocking CGRP. Intratracheally administered lipocalin mutein displays faster onset than subcunateously administered lipocalin mutein and comparable or even faster onset as compared to intravenously administered lipocalin mutein or reference antibody.

FIG. 5: provides the results of sensory nerve-mediated vasodilatation in rat treated with an exemplary lipocalin mutein (SEQ ID NO: 47) after intratracheal administration at a dose of 2.5 mg/kg, 5 mg/kg, or 10 mg mg/kg (FIG. 5A), or intravenous administration at a dose of 1 mg/kg, 2.5 mg/kg, 5 mg/kg, or 10 mg/kg (FIG. 5B), as described in Example 4. As a control, a reference anti-CGRP antibody (SEQ ID NOs: 204 and 205) was also tested via intravenous administration. In lipocalin mutein-treated animals, the dermal blood flow in the dorsomedial skin of the rat hind paw after nerve stimulation is significantly decreased from that seen in untreated animals, indicating increased vasoconstriction through blocking CGRP. The intratracheally-administered lipocalin mutein SEQ ID NO: 47 shows a rapid onset (within minutes) and durable potency over the two-hour study period in inhibiting vasodilation.

FIG. 6: provides the blood plasma lipocalin mutein concentration in rat treated with an exemplary lipocalin mutein (SEQ ID NO: 47) after intratracheal administration at a dose of 2.5 mg/kg, 5 mg/kg, or 10 mg mg/kg (FIG. 6A), or intravenous administration at a dose of 1 mg/kg, 2.5 mg/kg, 5 mg/kg, or 10 mg/kg (FIG. 6B), as described in Example 4.

### IV. DETAILED DESCRIPTION OF THE DISCLOSURE

The present invention is based on the surprising finding that a lipocalin mutein that is administered by inhalation results in local exposure in the respiratory tract, in particular in the lung. Inhaled drugs generally allow for a lower dose than is necessary with systemic delivery (oral or injection), and thus carry a lower risk profile, with the potential for fewer and less severe adverse effects (Bodier-Montagutelli et al., Expert Opin Drug Deliv, 2018). Other advantages of inhaled drugs include easier self-administration and better patient compliance (compared with injection) and faster mode of action. Systemic diffusion following topical delivery also occurs in some cases, and provides therapeutic benefit. The present invention is also based on the surprising finding that a lipocalin mutein that is administered by inhalation may also result in systemic exposure. Without wishing to be bound by theory it is believed that whether a lipocalin mutein enters the systemic circulation or whether systemic exposure can be detected depends inter alia on the dose of the lipocalin mutein that is administered or delivered to the lung.

The present invention is also based on the surprising finding that systemic administration of a lipocalin mutein by inhalation enables rapid delivery of lipocalin muteins to the circulatory system. It has been surprisingly found that the maximum concentration of lipocalin muteins in blood plasma can be reached after about 0.1 to about 10 hours after administration of the lipocalin mutein, preferably about 0.5 hours to about 5 hours after administration, preferably after about 1 hours to about 2 hours after administration.

The present invention is also based on the surprising finding that high levels of systemic exposure of lipocalin muteins (single- or double-digit percentages of the delivered dose) can be achieved by inhaled administration of such lipocalin muteins. Such high levels are surprising since WO 2013/087660 discloses an experiment in which only 0.2% of a lipocalin mutein that has been intratracheally administered to a mouse was detected in blood one hour after administration.

The present invention is also based on the surprising finding that a local administration to the lung without detectable systemic exposure of the lipocalin mutein is also achievable depending on the dose of the lipocalin mutein. This is particularly advantageous if the therapeutic effect of the lipocalin mutein is to be achieved locally in the lung and systemic exposure to the lipocalin mutein is not required or even undesired.

Accordingly, the present invention relates to a method of administration of a lipocalin mutein to a subject, wherein the method comprises administering the lipocalin mutein by inhalation, wherein the administration provides for local exposure to the lipocalin mutein in the respiratory tract.

The present invention also relates to a lipocalin mutein for use in therapy of a subject, wherein the use comprises administering the lipocalin mutein by inhalation, wherein the administration provides for local exposure to the lipocalin mutein in the respiratory tract.

The present invention also relates to the use of a lipocalin mutein for the preparation of a medicament for inhaled administration, wherein inhaled administration provides for local exposure to the lipocalin mutein in the respiratory tract.

The present invention also relates to a method of administration of a lipocalin mutein to a subject, wherein the method comprises administering the lipocalin mutein by inhalation, wherein the administration provides for systemic exposure to the lipocalin mutein.

The present invention also relates to a lipocalin mutein for use in therapy of a subject, wherein the use comprises administering the lipocalin mutein by inhalation, wherein the administration provides for systemic exposure to the lipocalin mutein.

The present invention also relates to the use of a lipocalin mutein for the preparation of a medicament for inhaled administration, wherein inhaled administration provides for systemic exposure to the lipocalin mutein.

A. Lipocalin Muteins of the Disclosure

As used herein, a “lipocalin” is defined as a monomeric protein of approximately 18-20 kDa in weight, having a cylindrical β-pleated sheet supersecondary structural region comprising a plurality of (preferably eight) β-strands connected pair-wise by a plurality of (preferably four) loops at one end to define thereby a binding pocket. It is the diversity of the loops in the otherwise rigid lipocalin scaffold that gives rise to a variety of different binding modes among the lipocalin family members, each capable of accommodating targets of different size, shape, and chemical character (reviewed, e.g. in Skerra, Biochim Biophys Acta, 2000, Flower et al., Biochim Biophys Acta, 2000, Flower, Biochem J, 1996). Indeed, the lipocalin family of proteins have naturally evolved to bind a wide spectrum of ligands, sharing unusually low levels of overall sequence conservation (often with sequence identities of less than 20%) yet retaining a highly conserved overall folding pattern. The correspondence between positions in various lipocalins is well known to one of skill in the art (see, e.g. U.S. Pat. No. 7,250,297).

As noted above, a lipocalin is a polypeptide defined by its supersecondary structure, namely cylindrical β-pleated sheet supersecondary structural region comprising eight β-strands connected pair-wise by four loops at one end to define thereby a binding pocket. The present disclosure is not limited to lipocalin muteins specifically disclosed herein. In this regard, the disclosure relates to lipocalin muteins having a cylindrical β-pleated sheet supersecondary structural region comprising eight β-strands connected pair-wise by four loops at one end to define thereby a binding pocket, wherein at least one amino acid of each of at least three of said four loops has been mutated as compared to the reference sequence, and wherein said lipocalin is effective to bind its target with detectable affinity.

A lipocalin mutein according to the present disclosure may be a mutein of any lipocalin. Examples of suitable lipocalins (also sometimes designated as “reference lipocalin,” “wild-type lipocalin,” “reference protein scaffolds,” or simply “scaffolds”) of which a mutein may be used include, but are not limited to, tear lipocalin (lipocalin-1, Tlc, or von Ebner's gland protein), retinol binding protein, neutrophil lipocalin-type prostaglandin D-synthase, β-lactoglobulin, bilin-binding protein (BBP), apolipoprotein D (APOD), neutrophil gelatinase-associated lipocalin (NGAL), α2-microglobulin-related protein (A2m), 24p3/uterocalin (24p3), von Ebner's gland protein 1 (VEGP 1), von Ebner's gland protein 2 (VEGP 2), and Major allergen Can f 1 (ALL-1). In related embodiments, a lipocalin mutein is derived from the lipocalin group consisting of human tear lipocalin (hTlc), human neutrophil gelatinase-associated lipocalin (hNGAL), human apolipoprotein D (hAPOD) and the bilin-binding protein of Pieris brassicae.

The amino acid sequence of a lipocalin mutein according to the disclosure may have a high sequence identity as compared to the reference (or wild-type) lipocalin from which it is derived, for example, hTlc or hNGAL, when compared to sequence identities with another lipocalin (see also above). In this general context the amino acid sequence of a lipocalin mutein according to the disclosure is at least substantially similar to the amino acid sequence of the corresponding reference (wild-type) lipocalin, with the proviso that there may be gaps (as defined herein) in an alignment that are the result of additions or deletions of amino acids. A respective sequence of a lipocalin mutein of the disclosure, being substantially similar to the sequences of the corresponding reference (wild-type) lipocalin, has, in some embodiments, at least 60%, at least 65%, at least 70%, at least 75%, at least 80%, at least 82%, at least 85%, at least 87%, at least 90% identity, including at least 95% identity to the sequence of the corresponding lipocalin. In this regard, a lipocalin mutein of the disclosure of course may contain substitutions as described herein which renders the lipocalin mutein capable of binding to a selected target.

Typically, a lipocalin mutein contains one or more mutated amino acid residues relative to the amino acid sequence of the wild-type or reference lipocalin, for example, hTlc and hNGAL in the four loops at the open end that comprise a ligand-binding pocket and define the entrance of ligand-binding pocket (cf. above). As explained above, these regions are essential in determining the binding specificity of a lipocalin mutein for the desired target. In some embodiments, a lipocalin mutein of the disclosure may also contain mutated amino acid residues regions outside of the four loops. In some embodiments, a lipocalin mutein of the disclosure may contain one or more mutated amino acid residues in one or more of the three peptide loops (designated BC, DE, and FG) connecting the β-strands at the closed end of the lipocalin. In some embodiments, a mutein derived from of tear lipocalin, NGAL lipocalin or a homologue thereof, may have 1, 2, 3, 4, or more mutated amino acid residues at any sequence position in the N-terminal region and/or in the three peptide loops BC, DE, and FG arranged at the end of the β-barrel structure that is located opposite to the natural lipocalin binding pocket. In some embodiments, a mutein derived from tear lipocalin, NGAL lipocalin or a homologue thereof, may have no mutated amino acid residues in peptide loop DE arranged at the end of the β-barrel structure, compared to wild-type sequence of tear lipocalin.

Any types and numbers of mutations, including substitutions, deletions, and insertions, are envisaged as long as a provided lipocalin mutein retains its capability to bind its target, and/or it has a sequence identity that it is at least 60%, such as at least 65%, at least 70%, at least 75%, at least 80%, at least 85% or higher identity to the amino acid sequence of the reference (wild-type) lipocalin, for example, mature hTlc or mature hNGAL. In some embodiments, a substitution is a conservative substitution. In some embodiments, a substitution is a non-conservative substitution.

Specifically, in order to determine whether an amino acid residue of the amino acid sequence of a lipocalin mutein is different from a reference (wild-type) lipocalin corresponds to a certain position in the amino acid sequence of the reference (wild-type) lipocalin, a skilled artisan can use means and methods well-known in the art, e.g., alignments, either manually or by using computer programs such as BLAST2.0, which stands for Basic Local Alignment Search Tool or ClustalW or any other suitable program which is suitable to generate sequence alignments. Accordingly, the amino acid sequence of a reference (wild-type) lipocalin can serve as “subject sequence” or “reference sequence”, while the amino acid sequence of a lipocalin mutein serves as “query sequence” (see also above).

Conservative substitutions are generally the following substitutions, listed according to the amino acid to be mutated, each followed by one or more replacement(s) that can be taken to be conservative: Ala→Ser, Thr, or Val; Arg→Lys, Gln, Asn, or His; Asn→Gln, Glu, Asp, or His; Asp→Glu, Gln, Asn, or His; Gln→Asn, Asp, Glu, or His; Glu→Asp, Asn, Gln, or His; His→Arg, Lys, Asn, Gln, Asp, or Glu; Ile→Thr, Leu, Met, Phe, Val, Trp, Tyr, Ala, or Pro; Leu→Thr, Ile, Val, Met, Ala, Phe, Pro, Tyr, or Trp; Lys→Arg, His, Gln, or Asn; Met→Thr, Leu, Tyr, Ile, Phe, Val, Ala, Pro, or Trp; Phe→Thr, Met, Leu, Tyr, Ile, Pro, Trp, Val, or Ala; Ser→Thr, Ala, or Val; Thr→Ser, Ala, Val, Ile, Met, Val, Phe, Pro, or Leu; Trp→Tyr, Phe, Met, Ile, or Leu; Tyr→Trp, Phe, Ile, Leu, or Met; Val→Thr, Ile, Leu, Met, Phe, Ala, Ser, or Pro. Other substitutions are also permissible and can be determined empirically or in accord with other known conservative or non-conservative substitutions. As a further orientation, the following groups each contain amino acids that can typically be taken to define conservative substitutions for one another: (a) Alanine (Ala), Serine (Ser), Threonine (Thr), Valine (Val); (b) Aspartic acid (Asp), Glutamic acid (Glu), Glutamine (Gln), Asparagine (Asn), Histidine (His); (c) Arginine (Arg), Lysine (Lys), Glutamine (Gln), Asparagine (Asn), Histidine (His); (d) Isoleucine (Ile), Leucine (Leu), Methionine (Met), Valine (Val), Alanine (Ala), Phenylalanine (Phe), Threonine (Thr), Proline (Pro); and (e) Isoleucine (Ile), Leucine (Leu), Methionine (Met), Phenylalanine (Phe), Tyrosine (Tyr), Tryptophan (Trp).

If such substitutions result in a change in biological activity, then more substantial changes, such as the following, or as further described below in reference to amino acid classes, may be introduced and the products screened for a desired characteristic. Examples of such more substantial changes are: Ala→Leu or Phe; Arg→Glu; Asn→Ile, Val, or Trp; Asp→Met; Cys→Pro; Gln→Phe; Glu→Arg; His→Gly; Ile→Lys, Glu, or Gln; Leu→Lys or Ser; Lys→Tyr; Met→Glu; Phe→Glu, Gln, or Asp; Trp→Cys; Tyr→Glu or Asp; Val→Lys, Arg, His.

In some embodiments, substantial modifications in the physical and biological properties of the lipocalin (mutein) are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain.

Naturally occurring residues are divided into groups based on common side-chain properties: (1) hydrophobic: methionine, alanine, valine, leucine, iso-leucine; (2) neutral hydrophilic: cysteine, serine, threonine, asparagine, glutamine; (3) acidic: aspartic acid, glutamic acid; (4) basic: histidine, lysine, arginine; (5) residues that influence chain orientation: glycine, proline; and (6) aromatic: tryptophan, tyrosine, phenylalanine. In some embodiments. substitutions may entail exchanging a member of one of these classes for another class.

Non-conservative substitutions will entail exchanging a member of one of these classes for another class. Any cysteine residue not involved in maintaining the proper conformation of the respective lipocalin also may be substituted, generally with serine, to improve the oxidative stability of the molecule and prevent aberrant crosslinking. Conversely, cysteine bond (s) may be added to the lipocalin to improve its stability.

Any cysteine residue not involved in maintaining the proper conformation of the respective lipocalin also may be substituted, generally with serine, to improve the oxidative stability of the molecule and prevent aberrant crosslinking. Conversely, cysteine bond (s) may be added to the lipocalin to improve its stability.

In some embodiments, lipocalin muteins disclosed herein may be or comprise a mutein of mature human tear lipocalin (hTlc). A mutein of mature hTlc may be designated herein as an “hTlc mutein”. In some other embodiments, a lipocalin mutein disclosed herein is a mutein of mature human neutrophil gelatinase-associated lipocalin (hNGAL). A mutein of mature hNGAL may be designated herein as an “hNGAL mutein”.

Any mutation, including an insertion as discussed above, can be accomplished very easily on the nucleic acid, e.g. DNA level using established standard methods. Illustrative examples of alterations of the amino acid sequence are insertions or deletions as well as amino acid substitutions. In addition, instead of replacing single amino acid residues, it is also possible to either insert or delete one or more continuous amino acids of the primary structure of the lipocalin (mutein) as long as these deletions or insertion result in a stable folded/functional mutein.

Modifications of the amino acid sequence include directed mutagenesis of single amino acid positions in order to simplify sub-cloning of the mutated lipocalin gene or its parts by incorporating cleavage sites for certain restriction enzymes. In addition, these mutations can also be incorporated to further improve the affinity of a lipocalin mutein for a given target. Furthermore, mutations can be introduced in order to modulate certain characteristics of the mutein such as to improve folding stability, serum stability, protein resistance or water solubility or to reduce aggregation tendency, if necessary. For example, naturally occurring cysteine residues may be mutated to other amino acids to prevent disulphide bridge formation. It is also possible to deliberately mutate other amino acid sequence positions to cysteine in order to introduce new reactive groups, for example for the conjugation to other compounds, such as polyethylene glycol (PEG), hydroxyethyl starch (HES), biotin, peptides or proteins, or for the formation of non-naturally occurring disulphide linkages. The generated thiol moiety may be used to PEGylate or HESylate the mutein, for example, in order to increase the serum half-life of a respective lipocalin mutein. Exemplary possibilities of such a mutation to introduce a cysteine residue into the amino acid sequence of a hTlc mutein include the substitutions Thr 40→Cys, Glu 73→Cys, Arg 90→Cys, Asp 95→Cys, and Glu 131→Cys. Similarly, with respect to a mutein of human NGAL, exemplary possibilities of introducing a cysteine residue into the amino acid sequence of the lipocalin mutein includes the introduction of a cysteine (Cys) residue at least at one of the sequence positions that correspond to sequence positions 14, 21, 60, 84, 88, 116, 141, 145, 143, 146 or 158 of the wild type sequence of human NGAL. The generated thiol moiety at the side of any of the above-mentioned amino acid positions may be used to PEGylate or HESylate the mutein, for example, in order to increase the serum half-life of a respective lipocalin mutein.

In another embodiment, in order to provide suitable amino acid side chains for conjugating one of the above compounds to a lipocalin mutein according to the present disclosure, artificial amino acids may be introduced by mutagenesis. Generally, such artificial amino acids are designed to be more reactive and thus to facilitate the conjugation to the desired compound. One example of such an artificial amino acid that may be introduced via an artificial tRNA is para-acetyl-phenylalanine.

For several applications of the muteins disclosed herein it may be advantageous to use them in the form of fusion proteins. In some embodiments, a lipocalin mutein of the disclosure is fused at its N-terminus or its C-terminus to a protein, a protein domain or a peptide, for instance, a signal sequence and/or an affinity tag.

Affinity tags such as the Strep-tag or Strep-tag II (Schmidt et al., J Mol Biol, 1996), the c-myc-tag, the FLAG-tag, the His-tag or the HA-tag or proteins such as glutathione-S-transferase, which allow easy detection and/or purification of recombinant proteins, are further examples of suitable fusion partners. Finally, proteins with chromogenic or fluorescent properties such as the green fluorescent protein (GFP) or the yellow fluorescent protein (YFP) are suitable fusion partners for lipocalin muteins of the disclosure as well.

In general, it is possible to label the lipocalin muteins of the disclosure with any appropriate chemical substance or enzyme, which directly or indirectly generates a detectable compound or signal in a chemical, physical, optical, or enzymatic reaction. An example for a physical reaction and at the same time optical reaction/marker is the emission of fluorescence upon irradiation or the emission of x-rays when using a radioactive label. Alkaline phosphatase, horseradish peroxidase and β-galactosidase are examples of enzyme labels (and at the same time optical labels) which catalyze the formation of chromogenic reaction products. In general, all labels commonly used for antibodies (except those exclusively used with the sugar moiety in the Fc part of immunoglobulins) can also be used for conjugation to the lipocalin muteins of the disclosure. The lipocalin muteins of the disclosure may also be conjugated with any suitable therapeutically active agent, e.g., for the targeted delivery of such agents to a given cell, tissue or organ, or for the selective targeting of cells (e.g. tumor cells) without affecting the surrounding normal cells. Examples of such therapeutically active agents include radionuclides, toxins, small organic molecules, and therapeutic peptides (such as peptides acting as agonists/antagonists of a cell surface receptor or peptides competing for a protein binding site on a given cellular target). The lipocalin muteins of the disclosure may, however, also be conjugated with therapeutically active nucleic acids such as antisense nucleic acid molecules, small interfering RNAs, micro RNAs or ribozymes. Such conjugates can be produced by methods well known in the art.

The disclosure also relates to a method for the production of a lipocalin mutein as described herein, wherein the mutein, a fragment of the mutein or a fusion protein of the mutein and another polypeptide is produced starting from the nucleic acid coding for the mutein by means of genetic engineering methods. The method can be carried out in vivo, the lipocalin mutein can for example be produced in a bacterial or eukaryotic host organism and then isolated from this host organism or its culture. It is also possible to produce a protein in vitro, for example by use of an in vitro translation system.

When producing the lipocalin mutein in vivo a nucleic acid encoding such mutein is introduced into a suitable bacterial or eukaryotic host organism by means of recombinant DNA technology (as already outlined above). For this purpose, the host cell is first transformed with a cloning vector that includes a nucleic acid molecule encoding a lipocalin mutein as described herein using established standard methods. The host cell is then cultured under conditions, which allow expression of the heterologous DNA and thus the synthesis of the corresponding polypeptide. Subsequently, the polypeptide is recovered either from the cell or from the cultivation medium.

In some embodiments, a nucleic acid molecule, such as DNA, disclosed in this application may be “operably linked” to another nucleic acid molecule of the disclosure to allow expression of a fusion protein of the disclosure. In this regard, an operable linkage is a linkage in which the sequence elements of the first nucleic acid molecule and the sequence elements of the second nucleic acid molecule are connected in a way that enables expression of the fusion protein as a single polypeptide.

In addition, in some embodiments for hTlc muteins of the disclosure, the naturally occurring disulfide bond between Cys 61 and Cys 153 may be removed. Accordingly, such muteins can be produced in a cell compartment having a reducing redox milieu, for example, in the cytoplasm of Gram-negative bacteria.

In case a lipocalin mutein of the disclosure includes intramolecular disulfide bonds, it may be preferred to direct the nascent polypeptide to a cell compartment having an oxidizing redox milieu using an appropriate signal sequence. Such an oxidizing environment may be provided by the periplasm of Gram-negative bacteria such as E. coli, in the extracellular milieu of Gram-positive bacteria or in the lumen of the endoplasmic reticulum of eukaryotic cells and usually favors the formation of structural disulfide bonds.

It is, however, also possible to produce a mutein of the disclosure in the cytosol of a host cell, preferably E. coli. In this case, the polypeptide can either be directly obtained in a soluble and folded state or recovered in form of inclusion bodies, followed by renaturation in vitro. A further option is the use of specific host strains having an oxidizing intracellular milieu, which may thus allow the formation of disulfide bonds in the cytosol (Venturi et al., J Mol Biol, 2002).

However, a lipocalin mutein as described herein may not necessarily be generated or produced only by use of genetic engineering. Rather, such a mutein can also be obtained by chemical synthesis such as Merrifield solid phase polypeptide synthesis or by in vitro transcription and translation. It is for example possible that promising mutations are identified using molecular modeling, polypeptides continuing such mutations synthesized in vitro, and investigated for binding activity with respect to its target and other desirable properties (such as stability). Methods for the solid phase and/or solution phase synthesis of polypeptides/proteins are well known in the art (see e.g. Bruckdorfer et al., Curr Pharm Biotechnol, 2004).

In another embodiment, the lipocalin muteins of the disclosure may be produced by in vitro transcription/translation employing well-established methods known to those skilled in the art.

The skilled worker will appreciate methods useful to prepare lipocalin muteins contemplated by the present disclosure but whose protein or nucleic acid sequences are not explicitly disclosed herein. As an overview, such modifications of the amino acid sequence include, e.g., directed mutagenesis of single amino acid positions in order to simplify sub-cloning of a mutated lipocalin gene or its parts by incorporating cleavage sites for certain restriction enzymes. In addition, these mutations can also be incorporated to further improve the affinity of a lipocalin mutein for its target. Furthermore, mutations can be introduced to modulate certain characteristics of the mutein such as to improve folding stability, serum stability, protein resistance or water solubility or to reduce aggregation tendency, if necessary. For example, naturally occurring cysteine residues may be mutated to other amino acids to prevent disulphide bridge formation.

The lipocalin muteins disclosed herein and its derivatives can be used in many fields similar to antibodies or fragments thereof. For example, the lipocalin muteins can be used for labeling with an enzyme, an antibody, a radioactive substance or any other group having biochemical activity or defined binding characteristics. By doing so, their respective targets or conjugates or fusion proteins thereof can be detected or brought in contact with them. In addition, lipocalin muteins of the disclosure can serve to detect chemical structures by means of established analytical methods (e.g., ELISA or Western Blot) or by microscopy or immunosensorics. In this regard, the detection signal can either be generated directly by use of a suitable mutein conjugate or fusion protein or indirectly by immunochemical detection of the bound mutein via an antibody.

1. Lipocalin Muteins Specific for IL4-Rα

Interleukin-4 receptor alpha chain (IL-4Rα) is a type I transmembrane protein that can bind interleukin 4 and interleukin 13 to regulate IgE antibody production in B cells. Among T cells, the encoded protein also can bind interleukin 4 to promote differentiation of Th2 cells.

Lipocalin muteins that are specific for IL-4 receptor, in particular human IL-4Rα are disclosed in International patent publications WO 2008/015239, WO 2011/154420, and WO 2013/087660. Inhaled administration of lipocalin muteins specific for human IL-4Rα have been reported by Bruns I B, Fitzgerald M F, Pardali K, Gardiner P, Keeling D J, Axelsson L T, Jiang F, Lickliter J, Close D R, First-in-human data for the inhaled IL-4Rα antagonist AZD1402/PRS-060 reveals a promising clinical profile for the treatment of asthma, presented at the American Thoracic Society Annual Congress, Dallas, Tex., USA, May 17-22, 2019, and Bruns I B, Fitzgerald M F, Pardali K, Gardiner P, Keeling D J, Axelsson L T, Jiang F, Lickliter J, Close D R, Phase 1 evaluation of the inhaled IL-4Rα antagonist AZD1402/PRS-060, a potent and selective blocker of the IL-4Rα, presented at the European Respiratory Society International Congress, Madrid, Spain, 28 Sep.-2 Oct., 2019, which are incorporated herewith by reference.

An IL-4Rα-specific lipocalin mutein of the disclosure may be a mutein of human tear lipocalin. As compared to the linear polypeptide sequence of mature human tear lipocalin (SEQ ID NO: 1), such mutein may comprise one of the following sets of mutated amino acid residues:
(a) Arg 26→Ser; Glu 27→Arg; Phe 28→Cys; Glu 30→Arg; Met 31→Ala; Asn 32→Tyr; Leu 33→Tyr; Glu 34→Gly; Leu 56→Gln; Ile 57→Arg; Ser 58→Ile; Asp 80→Ser; Lys 83→Arg; Glu 104→Leu; Leu 105→Cys; His 106→Pro; Lys 108→Gln;
(b) Arg 26→Ser; Glu 27→Arg; Phe 28→Cys; Glu 30→Arg; Met 31→Ala; Asn 32→Tyr; Leu 33→Tyr; Glu 34→Lys; Leu 56→Gln; Ile 57→Arg; Ser 58→Asn; Asp 80→Ser; Lys 83→Arg; Glu 104→Leu; Leu 105→Cys; His 106→Pro; Lys 108→Gln;
(c) Arg 26→Ser; Glu 27→Arg; Phe 28→Cys, Glu 30→Arg; Met 31→Ala; Asn 32→Tyr; Leu 33→Tyr; Leu 56→Gln; Ile 57→Arg; Ser 58→Arg; Asp 80→Ser; Lys 83→Arg; Glu 104→Leu; Leu 105→Cys; His 106→Pro; Lys 108→Gln;
(d) Arg 26→Ser; Glu 27→Arg; Phe 28→Cys; Glu 30→Arg; Met 31→Ala; Asn 32→Tyr; Leu 33→Tyr; Glu 34→Ser; Leu 56→Gln; Ile 57→Arg; Asp 80→Ser; Lys 83→Arg; Glu 104→Leu; Leu 105→Cys; His 106→Pro; Lys 108→Gln;
(e) Arg 26→Ser; Glu 27→Arg; Phe 28→Cys; Glu 30→Arg; Met 31→Ala; Asn 32→His; Leu 33→Tyr; Glu 34→Ser; Leu 56→Gln; Ile 57→Arg; Ser 58→Ala; Asp 80→Ser; Lys 83→Arg; Glu 104→Leu; Leu 105→Cys; His 106→Pro; Lys 108→Gln;
(f) Arg 26→Ser; Glu 27→Arg; Phe 28→Cys; Glu 30→Arg; Met 31→Ala; Asn 32→Tyr; Leu 33→Tyr; Glu 34→Asp; Leu 56→Gln; Ile 57→Arg; Ser 58→Lys; Asp 80→Ser; Lys 83→Arg; Glu 104→Leu; Leu 105→Cys; His 106→Pro; Lys 108→Gln;
(g) Arg 26→Ser; Glu 27→Arg; Phe 28→Cys; Glu 30→Arg; Met 31→Ala; Asn 32→Tyr; Leu 33→Tyr; Glu 34→Gly; Leu 56→Gln; Ile 57→Arg; Asp 80→Ser; Lys 83→Arg; Glu 104→Leu; Leu 105→Cys; His 106→Pro; Lys 108→Gln;
(h) Arg 26→Ser; Glu 27→Arg; Phe 28→Cys; Glu 30→Arg; Met 31→Ala; Asn 32→Tyr; Leu 33→Tyr; Glu 34→Gly; Leu 56→Gln; Ile 57→Arg; Ser 58→Ile; Asp 80→Ser; Lys 83→Arg; Glu 104→Leu; Leu 105→Cys; His 106→Pro; Lys 108→Gln;
(i) Arg 26→Ser; Glu 27→Arg; Phe 28→Cys; Glu 30→Arg; Met 31→Ala; Asn 32→Tyr; Leu 33→Tyr; Glu 34→Lys; Leu 56→Gln; Ile 57→Arg; Ser 58→Asn; Asp 80→Ser; Lys 83→Arg; Glu 104→Leu; Leu 105→Cys; His 106→Pro; Lys 108→Gln;
(j) Arg 26→Ser; Glu 27→Arg; Phe 28→Cys, Glu 30→Arg; Met 31→Ala; Asn 32→Tyr; Leu 33→Tyr; Leu 56→Gln; Ile 57→Arg; Ser 58→Arg; Asp 80→Ser; Lys 83→Arg; Glu 104→Leu; Leu 105→Cys; His 106→Pro; Lys 108→Gln;
(k) Arg 26→Ser; Glu 27→Arg; Phe 28→Cys; Glu 30→Arg; Met 31→Ala; Asn 32→Tyr; Leu 33→Tyr; Glu 34→Ser; Leu 56→Gln; Ile 57→Arg; Asp 80→Ser; Lys 83→Arg; Glu 104→Leu; Leu 105→Cys; His 106→Pro; Lys 108→Gln;
(l) Arg 26→Ser; Glu 27→Arg; Phe 28→Cys; Glu 30→Arg; Met 31→Ala; Asn 32→His; Leu 33→Tyr; Glu 34→Ser; Leu 56→Gln; Ile 57→Arg; Ser 58→Ala; Asp 80→Ser; Lys 83→Arg; Glu 104→Leu; Leu 105→Cys; His 106→Pro; Lys 108→Gln;
(m) Arg 26→Ser; Glu 27→Arg; Phe 28→Cys; Glu 30→Arg; Met 31→Ala; Asn 32→Tyr; Leu 33→Tyr; Glu 34→Asp; Leu 56→Gln; Ile 57→Arg; Ser 58→Lys; Asp 80→Ser; Lys 83→Arg; Glu 104→Leu; Leu 105→Cys; His 106→Pro; Lys 108→Gln; or
(n) Arg 26→Ser; Glu 27→Arg; Phe 28→Cys; Glu 30→Arg; Met 31→Ala; Asn 32→Tyr; Leu 33→Tyr; Glu 34→Gly; Leu 56→Gln; Ile 57→Arg; Asp 80→Ser; Lys 83→Arg; Glu 104→Leu; Leu 105→Cys; His 106→Pro; Lys 108→Gln.

An IL-4Rα-specific lipocalin mutein of the disclosure may comprise an amino acid sequence selected from the group consisting of SEQ ID NOs: 177-194, or a fragment or variant thereof, or a fragment or variant thereof. An IL-4Rα-specific lipocalin mutein of the disclosure may have at least 70%, at least 75%, at least 80%, at least 85%, at least 90%, at least 92%, at least 95%, at least 97%, at least 98%, or even higher sequence identity to the amino acid sequences shown in any one of SEQ ID NOs: 177-194.

2. Lipocalin Muteins Specific for CGRP

Calcitonin gene-related peptide (CGRP) is a vasoactive neuropeptide secreted by the nerves of the central and peripheral nervous systems, where CGRP-containing neurons are closely associated with blood vessels. CGRP-mediated vasodilatation is also associated with neurogenic inflammation, as part of a cascade of events that results in extravasation of plasma and vasodilatation of the microvasculature and is present in migraines.

Lipocalin muteins that are specific for CGRP are disclosed in International patent publication WO 2017/097946.

A CGRP-specific lipocalin mutein of the disclosure may be a mutein of hNGAL. As compared to the linear polypeptide sequence of mature hNGAL (SEQ ID NO: 2), such mutein may comprise one of the following sets of mutated amino acid residues:
(a) Gln 28→His; Leu 36→Glu; Ala 40→Trp; Ile 41→Gly; Gln 49→Lys; Tyr 52→Ala; Ser 68→Asp; Leu 70→Gln; Arg 72→Ile; Lys 73→Glu; Arg 81→Gly; Cys 87→Ser; Asn 96→Ala; Tyr 100→Glu; Leu 103→Gln; Tyr 106→Asn; Lys 125→Glu; Ser 127→Trp; Tyr 132→Leu; Lys 134→Trp;
(b) Gln 28→His; Leu 36→Phe; Ala 40→Met; Ile 41→Trp; Gln 49→Phe; Tyr 52→Gly; Ser 68→Trp; Leu 70→Trp; Arg 72→Glu; Lys 73→Ala; Trp 79→Gly; Arg 81→Asn; Cys 87→Ser; Asn 96→Gly; Tyr 100→Pro; Leu 103→Met; Tyr 106→His; Lys 125→Glu; Ser 127→Phe; Tyr 132→Trp; Lys 134→Trp;
(c) Gln 28→His; Leu 36→Trp; Ala 40→Thr; Ile 41→Leu; Gln 49→Thr; Tyr 52→Gln; Ser 68→Trp; Leu 70→Tyr; Arg 72→Ser; Lys 73→Glu; Asp 77→Asn; Trp 79→His; Arg 81→Glu; Cys 87→Ser; Asn 96→Thr; Leu 103→Glu; Tyr 106→Ile; Lys 125→Gly; Tyr 132→Ile; Lys 134→Glu;
(d) Gln 28→His; Leu 36→Arg; Ile 41→Glu; Gln 49→Glu; Tyr 52→Glu; Ser 68→Asp; Leu 70→Gly; Arg 72→Trp; Lys 73→Gln; Asp 77→Ile; Trp 79→Val; Arg 81→His; Cys 87→Ser; Leu 103→Thr; Tyr 106→Ala; Lys 125→Val; Ser 127→Arg; Tyr 132→Trp; Lys 134→Glu;
(e) Gln 28→His; Leu 36→Ile; Ala 40→Trp; Ile 41→Trp; Gln 49→Leu; Ser 68→His; Leu 70→Met; Arg 72→Met; Lys 73→Thr; Trp 79→Thr; Cys 87→Ser; Tyr 100→Ile; Leu 103→Met; Tyr 106→Leu; Lys 125→Phe; Ser 127→Trp; Tyr 132→Trp; Lys 134→His;
(f) Gln 28→His; Leu 36→Trp; Ala 40→Thr; Ile 41→Leu; Gln 49→Ile; Tyr 52→Gln; Ser 68→Trp; Leu 70→Tyr; Arg 72→Ser; Lys 73→Glu; Lys 75→Arg; Asp 77→Asn; Trp 79→His; Arg 81→Glu; Phe 83→Ser; Cys 87→Ser; Asn 96→Thr; Leu 103→Glu; Tyr 106→Ile; Lys 125→Gly; Tyr 132→Ile; Lys 134→Glu;
(g) Gln 28→His; Leu 36→Trp; Ala 40→Thr; Ile 41→Leu; Leu 42→Arg; Asp 47→Asn; Gln 49→Thr; Tyr 52→Gln; Ser 68→Trp; Leu 70→Tyr; Arg 72→Ser; Lys 73→Glu; Asp 77→Asn; Trp 79→His; Arg 81→Glu; Phe 83→Ser; Cys 87→Ser; Asn 96→Thr; Leu 103→Glu; Tyr 106→Ile; Lys 125→Gly; Tyr 132→Ile; Lys 134→Glu;
(h) Gln 28→His; Leu 36→Trp; Ala 40→Thr; Ile 41→Leu; Gln 49→Ile; Tyr 52→Gln; Asn 65→Asp; Ser 68→Trp; Leu 70→Tyr; Phe 71→Leu; Arg 72→Ser; Lys 73→Glu; Asp 77→Asn; Trp 79→His; Arg 81→Glu; Phe 83→Ser; Cys 87→Ser; Asn 96→Thr; Leu 103→Glu; Tyr 106→Ile; Lys 125→Gly; Val 126→Met; Tyr 132→Ile; Lys 134→Glu; Thr 145→Ala;
(i) Gln 28→His; Leu 36→Trp; Ala 40→Thr; Ile 41→Leu; Asp 47→Asn; Gln 49→Thr; Tyr 52→Gln; Val 66→Ala; Ser 68→Trp; Leu 70→Tyr; Phe 71→Leu; Arg 72→Ser; Lys 73→Glu; Asp 77→Asn; Trp 79→His; Arg 81→Glu; Phe 83→Ser; Cys 87→Ser; Asn 96→Thr; Ile 97→Thr; Leu 103→Glu; Ser 105→Pro; Tyr 106→Ile; Lys 125→Gly; Tyr 132→Ile; Lys 134→Glu;
(j) Gln 28→His; Leu 36→Trp; Ala 40→Thr; Ile 41→Leu; Gln 49→Ile; Tyr 52→Gln; Thr 54→Ile; Lys 62→Arg; Ser 68→Trp; Leu 70→Tyr; Arg 72→Ser; Lys 73→Glu; Asp 77→Asn; Trp 79→His; Arg 81→Glu; Cys 87→Ser; Asn 96→Thr; Leu 103→Glu; Ser 105→Pro; Tyr 106→Ile; Lys 125→Gly; Ser 127→Asn; Tyr 132→Ile; Lys 134→Glu; Thr 136→Ile; Ser 146→Asn;
(k) Gln 28→His; Leu 36→Trp; Ala 40→Thr; Ile 41→Leu; Gln 49→Pro; Tyr 52→Gln; Lys 62→Arg; Ser 68→Trp; Leu 70→Tyr; Arg 72→Ser; Lys 73→Glu; Asp 77→Asn; Trp 79→His; Arg 81→Glu; Phe 83→Ser; Cys 87→Ser; Asn 96→Thr; Lys 98→Gln; Tyr 100→His; Leu 103→Glu; Ser 105→Pro; Tyr 106→Ile; Lys 125→Gly; Val 126→Met; Ser 127→Gly; Tyr 132→Ile; Lys 134→Glu;
(l) Gln 28→His; Leu 36→Trp; Ala 40→Thr; Ile 41→Leu; Gln 49→Ile; Tyr 52→Gln; Thr 54→Lys; Lys 62→Arg; Ser 68→Trp; Leu 70→Tyr; Arg 72→Ala; Lys 73→Asp; Asp 77→Asn; Trp 79→His; Arg 81→Glu; Cys 87→Ser; Asn 96→Leu; Leu 103→Glu; Ser 105→Pro; Tyr 106→Ile; Lys 125→Gly; Ser 127→Asn; Tyr 132→Ile; Lys 134→Glu; Thr 136→Ile; Ser 146→Asn;
(m) Gln 28→His; Leu 36→Trp; Ala 40→Thr; Ile 41→Leu; Gln 49→Ile; Tyr 52→Gln; Thr 54→Lys; Lys 62→Arg; Ser 68→Trp; Leu 70→Tyr; Arg 72→Ala; Lys 73→Glu; Asp 77→Asn; Trp 79→His; Arg 81→Glu; Cys 87→Ser; Asn 96→Thr; Leu 103→Glu; Ser 105→Pro; Tyr 106→Ile; Lys 125→Gly; Ser 127→Asn; Tyr 132→Ile; Lys 134→Glu; Thr 136→Ile; Ser 146→Asn;
(n) Gln 28→His; Leu 36→Trp; Ala 40→Thr; Ile 41→Leu; Gln 49→Ile; Tyr 52→Gln; Thr 54→Lys; Lys 62→Arg; Ser 68→Trp; Leu 70→Tyr; Arg 72→Ala; Lys 73→Asp; Asp 77→Asn; Trp 79→His; Arg 81→Glu; Cys 87→Ser; Asn 96→Thr; Leu 103→Glu; Ser 105→Pro; Tyr 106→Ile; Lys 125→Gly; Ser 127→Asn; Tyr 132→Ile; Lys 134→Glu; Thr 136→Ile; Ser 146→Asn;
(o) Gln 28→His; Leu 36→Trp; Ala 40→Thr; Ile 41→Leu; Gln 49→Ile; Tyr 52→Gln; Thr 54→Lys; Lys 62→Arg; Ser 68→Trp; Leu 70→Tyr; Arg 72→Ala; Lys 73→Asp; Asp 77→Arg; Trp 79→His; Arg 81→Glu; Cys 87→Ser; Asn 96→Thr; Tyr 100→His; Leu 103→Glu; Ser 105→Pro; Tyr 106→Ile; Lys 125→Gly; Ser 127→Asn; Tyr 132→Ile; Lys 134→Glu; Thr 136→Ile; Ser 146→Asn;
(p) Gln 28→His; Leu 36→Trp; Ala 40→Thr; Ile 41→Leu; Gln 49→Ile; Tyr 52→Gln; Thr 54→Met; Ser 68→Trp; Leu 70→Tyr; Arg 72→Ala; Lys 73→Asp; Asp 77→Asn; Trp 79→His; Arg 81→Glu; Cys 87→Ser; Asn 96→Thr; Leu 103→Glu; Ser 105→Pro; Tyr 106→Ile; Lys 125→Gly; Ser 127→Asn; Tyr 132→Ile; Lys 134→Glu; Thr 136→Val; Ser 146→Asn;
(q) Gln 28→His; Leu 36→Trp; Ala 40→Thr; Ile 41→Leu; Gln 49→Ile; Tyr 52→Gln; Thr 54→Ile; Lys 62→Arg; Ser 68→Trp; Leu 70→Tyr; Arg 72→Ser; Lys 73→Glu; Asp 77→Asn; Trp 79→His; Arg 81→Glu; Cys 87→Ser; Asn 96→Thr; Leu 103→Glu; Ser 105→Pro; Tyr 106→Ile; Val 111→Met; Lys 125→Gly; Ser 127→Asn; Tyr 132→Ile; Lys 134→Glu; Thr 136→Ile; Ser 146→Asn;
(r) Leu 36→Trp; Ala 40→Thr; Ile 41→Leu; Gln 49→Ile; Tyr 52→Gln; Thr 54→Met; Ser 68→Trp; Leu 70→Tyr; Arg 72→Ala; Lys 73→Asp; Cys 76→Arg; Asp 77→Asn; Trp 79→His; Arg 81→Glu; Cys 87→Ser; Asn 96→Thr; Leu 103→Glu; Ser 105→Pro; Tyr 106→Ile; Lys 125→Gly; Ser 127→Asn; Tyr 132→Ile; Lys 134→Glu; Thr 136→Val; Ser 146→Asn; Cys 175→Phe;
(s) Leu 36→Trp; Ala 40→Thr; Ile 41→Leu; Gln 49→Ile; Tyr 52→Gln; Thr 54→Met; Ser 68→Trp; Leu 70→Tyr; Arg 72→Ala; Lys 73→Asp; Cys 76→Met; Asp 77→Asn; Trp 79→His; Arg 81→Glu; Cys 87→Ser; Asn 96→Thr; Leu 103→Glu; Ser 105→Pro; Tyr 106→Ile; Lys 125→Gly; Ser 127→Asn; Tyr 132→Ile; Lys 134→Glu; Thr 136→Val; Ser 146→Asn; Cys 175→Tyr;
(t) Leu 36→Trp; Ala 40→Thr; Ile 41→Leu; Gln 49→Ile; Tyr 52→Gln; Thr 54→Met; Ser 68→Trp; Leu 70→Tyr; Arg 72→Ala; Lys 73→Asp; Cys 76→Leu; Asp 77→Asn; Trp 79→His; Arg 81→Glu; Cys 87→Ser; Asn 96→Thr; Leu 103→Glu; Ser 105→Pro; Tyr 106→Ile; Lys 125→Gly; Ser 127→Asn; Tyr 132→Ile; Lys 134→Glu; Thr 136→Val; Ser 146→Asn; Cys 175→Trp;
(u) Leu 36→Trp; Ala 40→Thr; Ile 41→Leu; Gln 49→Ile; Tyr 52→Gln; Thr 54→Met; Ser 68→Trp; Leu 70→Tyr; Arg 72→Ala; Lys 73→Asp; Cys 76→Ile; Asp 77→Asn; Trp 79→His; Arg 81→Glu; Cys 87→Ser; Asn 96→Thr; Leu 103→Glu; Ser 105→Pro; Tyr 106→Ile; Lys 125→Gly; Ser 127→Asn; Tyr 132→Ile; Lys 134→Glu; Thr 136→Val; Ser 146→Asn; Cys 175→Glu;
(v) Leu 36→Trp; Ala 40→Thr; Ile 41→Leu; Gln 49→Ile; Tyr 52→Gln; Thr 54→Met; Ser 68→Trp; Leu 70→Tyr; Arg 72→Ala; Lys 73→Asp; Cys 76→Val; Asp 77→Asn; Trp 79→His; Arg 81→Glu; Cys 87→Ser; Asn 96→Thr; Leu 103→Glu; Ser 105→Pro; Tyr 106→Ile; Lys 125→Gly; Ser 127→Asn; Tyr 132→Ile; Lys 134→Glu; Thr 136→Val; Ser 146→Asn; Cys 175→Tyr;
(w) Leu 36→Trp; Ala 40→Thr; Ile 41→Leu; Gln 49→Ile; Tyr 52→Gln; Thr 54→Met; Ser 68→Trp; Leu 70→Tyr; Arg 72→Ala; Lys 73→Asp; Cys 76→Arg; Asp 77→Asn; Trp 79→His; Arg 81→Glu; Cys 87→Ser; Asn 96→Thr; Leu 103→Glu; Ser 105→Pro; Tyr 106→Ile; Lys 125→Gly; Ser 127→Asn; Tyr 132→Ile; Lys 134→Glu; Thr 136→Val; Ser 146→Asn; Cys 175→Trp;
(x) Leu 36→Trp; Ala 40→Thr; Ile 41→Leu; Gln 49→Ile; Tyr 52→Gln; Thr 54→Met; Ser 68→Trp; Leu 70→Tyr; Arg 72→Ala; Lys 73→Asp; Cys 76→Asn; Asp 77→Asn; Trp 79→His; Arg 81→Glu; Cys 87→Ser; Asn 96→Thr; Leu 103→Glu; Ser 105→Pro; Tyr 106→Ile; Lys 125→Gly; Ser 127→Asn; Tyr 132→Ile; Lys 134→Glu; Thr 136→Val; Ser 146→Asn; Cys 175→Leu;
(y) Leu 36→Trp; Ala 40→Thr; Ile 41→Leu; Gln 49→Ile; Tyr 52→Gln; Thr 54→Met; Ser 68→Trp; Leu 70→Tyr; Arg 72→Ala; Lys 73→Asp; Cys 76→Arg; Asp 77→Asn; Trp 79→His; Arg 81→Glu; Cys 87→Ser; Asn 96→Thr; Leu 103→Glu; Ser 105→Pro; Tyr 106→Ile; Lys 125→Gly; Ser 127→Asn; Tyr 132→Ile; Lys 134→Glu; Thr 136→Val; Ser 146→Asn; Cys 175→Val;
(z) Leu 36→Trp; Ala 40→Thr; Ile 41→Leu; Gln 49→Ile; Tyr 52→Gln; Thr 54→Met; Ser 68→Trp; Leu 70→Tyr; Arg 72→Ala; Lys 73→Asp; Cys 76→Lys; Asp 77→Asn; Trp 79→His; Arg 81→Glu; Cys 87→Ser; Asn 96→Thr; Leu 103→Glu; Ser 105→Pro; Tyr 106→Ile; Lys 125→Gly; Ser 127→Asn; Tyr 132→Ile; Lys 134→Glu; Thr 136→Val; Ser 146→Asn; Cys 175→Asp;
(aa) Leu 36→Trp; Ala 40→Thr; Ile 41→Leu; Gln 49→Ile; Tyr 52→Gln; Thr 54→Met; Ser 68→Trp; Leu 70→Tyr; Arg 72→Ala; Lys 73→Asp; Cys 76→Phe; Asp 77→Asn; Trp 79→His; Arg 81→Glu; Cys 87→Ser; Asn 96→Thr; Leu 103→Glu; Ser 105→Pro; Tyr 106→Ile; Lys 125→Gly; Ser 127→Asn; Tyr 132→Ile; Lys 134→Glu; Thr 136→Val; Ser 146→Asn; Cys 175→Asp;
(bb) Gln 28→His; Leu 36→Arg; Ala 40→Glu; Ile 41→Glu; Tyr 52→Glu; Ser 68→Asp; Leu 70→Gly; Arg 72→Ser; Lys 73→Glu; Asp 77→Ile; Trp 79→Val; Arg 81→His; Cys 87→Ser; Leu 103→Val; Tyr 106→Ala; Lys 125→Val; Ser 127→Lys; Tyr 132→Leu; Lys 134→Glu;
(cc) Gln 28→His; Leu 36→Arg; Ala 40→Asp; Ile 41→Ala; Gln 49→Glu; Tyr 52→Glu; Ser 68→Asp; Leu 70→Gly; Arg 72→Val; Lys 73→Gln; Asp 77→Met; Trp 79→Val; Arg 81→His; Cys 87→Ser; Leu 103→Val; Tyr 106→Ala; Lys 125→Leu; Ser 127→Lys; Tyr 132→Leu; Lys 134→Glu;
(dd) Gln 28→His; Leu 36→Arg; Gly 38→Ala; Ala 40→Asp; Ile 41→Arg; Gln 49→Glu; Tyr 52→Glu; Ser 68→Asp; Leu 70→Gly; Arg 72→Ser; Lys 73→Arg; Asp 77→Ile; Trp 79→Val; Arg 81→His; Cys 87→Ser; Leu 103→Thr; Tyr 106→Gly; Lys 125→Val; Ser 127→Gly; Tyr 132→Ser; Lys 134→Glu;
(ee) Gln 28→His; Leu 36→Arg; Ala 40→Asp; Ile 41→Glu; Gln 49→Glu; Tyr 52→Glu; Ser 68→Asp; Leu 70→Gly; Arg 72→Val; Lys 73→Gln; Asp 77→Met; Trp 79→Val; Arg 81→His; Cys 87→Ser; Leu 103→Val; Tyr 106→Gly; Lys 125→Val; Ser 127→Arg; Tyr 132→Leu; Lys 134→Glu;
(ff) Gln 28→His; Leu 36→Arg; Ala 40→Asp; Ile 41→Val; Gln 49→Glu; Tyr 52→Glu; Ser 68→Asp; Leu 70→Gly; Arg 72→Val; Lys 73→Gln; Lys 75→Arg; Asp 77→Met; Trp 79→Val; Ile 80→Thr; Arg 81→His; Cys 87→Ser; Lys 98→Glu; Leu 103→Val; Tyr 106→Ala; Asn 114→Asp; Phe 123→Val; Lys 125→Leu; Ser 127→Lys; Tyr 132→Leu; Lys 134→Glu;
(gg) Gln 28→His; Leu 36→Arg; Gly 38→Ala; Ala 40→Asp; Ile 41→Val; Glu 44→Asp; Lys 46→Asn; Gln 49→Glu; Tyr 52→Glu; Ser 68→Asp; Leu 70→Gly; Arg 72→Val; Lys 73→Gln; Asp 77→Met; Trp 79→Val; Ile 80→Val; Arg 81→His; Cys 87→Ser; Leu 103→Val; Tyr 106→Ala; Lys 125→Leu; Ser 127→Lys; Tyr 132→Leu; Lys 134→Glu; Ile 135→Val;
(hh) Gln 28→His; Leu 36→Arg; Ala 40→Asp; Ile 41→Thr; Gln 49→Glu; Tyr 52→Glu; Ser 68→Asp; Leu 70→Gly; Phe 71→Leu; Arg 72→Val; Lys 73→Gln; Asp 77→Met; Trp 79→Val; Arg 81→His; Cys 87→Ser; Leu 103→Val; Tyr 106→Ala; Phe 123→Val; Lys 125→Leu; Ser 127→Lys; Asn 129→Ser; Tyr 132→Leu; Lys 134→Glu;
(ii) Gln 28→His; Leu 36→Arg; Ala 40→Asp; Ile 41→Thr; Glu 44→Lys; Gln 49→Glu; Tyr 52→Glu; Ser 68→Asp; Leu 70→Gly; Arg 72→Val; Lys 73→Gln; Asp 77→Met; Trp 79→Val; Arg 81→His; Cys 87→Ser; Leu 103→Val; Tyr 106→Ala; Phe 123→Val; Lys 125→Leu; Ser 127→Lys; Tyr 132→Leu; Lys 134→Glu;
(jj) Gln 28→His; Leu 36→Arg; Ala 40→Asp; Ile 41→Ala; Gln 49→Glu; Tyr 52→Glu; Ser 68→Asp; Leu 70→Gly; Arg 72→Val; Lys 73→Gln; Asp 77→Met; Trp 79→Val; Arg 81→His; Cys 87→Ser; Leu 103→Val; Tyr 106→Ala; Val 108→Ile; Ser 112→Asn; Phe 123→Val; Lys 125→Leu; Ser 127→Lys; Tyr 132→Leu; Lys 134→Glu;
(kk) Gln 28→His; Leu 36→Arg; Ala 40→Asp; Ile 41→Ala; Gln 49→Glu; Tyr 52→Glu; Ser 68→Asp; Leu 70→Gly; Phe 71→Leu; Arg 72→Val; Lys 73→Gln; Asp 77→Met; Trp 79→Val; Arg 81→His; Cys 87→Gly; Leu 103→Val; Tyr 106→Ala; Phe 123→Val; Lys 125→Leu; Ser 127→Lys; Tyr 132→Leu; Lys 134→Glu;
(ll) Leu 36→Arg; Ala 40→Asp; Ile 41→Val; Gln 49→Glu; Tyr 52→Glu; Ser 68→Asp; Leu 70→Gly; Arg 72→Val; Lys 73→Gln; Lys 75→Arg; Cys 76→Leu; Asp 77→Met; Trp 79→Val; Ile 80→Thr; Arg 81→His; Cys 87→Ser; Lys 98→Glu; Leu 103→Val; Tyr 106→Ala; Asn 114→Asp; Phe 123→Val; Lys 125→Leu; Ser 127→Lys; Tyr 132→Leu; Lys 134→Glu; Cys 175→Ile; Ile 176→Asp; Asp 177→Gly;
(mm) Leu 36→Arg; Ala 40→Asp; Ile 41→Val; Gln 49→Glu; Tyr 52→Glu; Ser 68→Asp; Leu 70→Gly; Arg 72→Val; Lys 73→Gln; Lys 75→Arg; Cys 76→Tyr; Asp 77→Met; Trp 79→Val; Ile 80→Thr; Arg 81→His; Cys 87→Ser; Lys 98→Glu; Leu 103→Val; Tyr 106→Ala; Asn 114→Asp; Phe 123→Val; Lys 125→Leu; Ser 127→Lys; Tyr 132→Leu; Lys 134→Glu; Cys 175→Ile; Ile 176→Asp; Asp 177→Gly;
(nn) Leu 36→Trp; Ala 40→Thr; Ile 41→Leu; Gln 49→Ile; Tyr 52→Gln; Thr 54→Met; Ser 68→Trp; Leu 70→Tyr; Arg 72→Ala; Lys 73→Asp; Cys 76→Ile; Asp 77→Asn; Trp 79→His; Arg 81→Glu; Cys 87→Ser; Asn 96→Thr; Leu 103→Glu; Ser 105→Pro; Tyr 106→Ile; Lys 125→Gly; Ser 127→Asn; Tyr 132→Ile; Lys 134→Glu; Thr 136→Val; Ser 146→Asn; Cys 175→Glu; and, optionally, a Gly residue is added N-terminally;
(oo) Gln 28→His; Leu 36→Arg; Ala 40→Asp; Ile 41→Val; Gln 49→Glu; Tyr 52→Glu; Ser 68→Asp; Leu 70→Gly; Arg 72→Val; Lys 73→Gln; Lys 75→Arg; Asp 77→Met; Trp 79→Val; Ile 80→Thr; Arg 81→His; Cys 87→Ser; Lys 98→Glu; Leu 103→Val; Tyr 106→Ala; Asn 114→Asp; Phe 123→Val; Lys 125→Leu; Ser 127→Lys; Tyr 132→Leu; Lys 134→Glu; and, optionally, a Gly residue is added N-terminally;
(pp) Gln 28→His; Leu 36→Arg; Ala 40→Asp; Ile 41→Val; Gln 49→Glu; Tyr 52→Glu; Asn 65→Gln; Ser 68→Asp; Leu 70→Gly; Arg 72→Val; Lys 73→Gln; Lys 75→Arg; Asp 77→Met; Trp 79→Val; Ile 80→Thr; Arg 81→His; Cys 87→Ser; Lys 98→Glu; Leu 103→Val; Tyr 106→Ala; Asn 114→Asp; Phe 123→Val; Lys 125→Leu; Ser 127→Lys; Tyr 132→Leu; Lys 134→Glu; Gly 178→Asp; and, optionally, a Gly residue is added N-terminally;
(qq) Ile 8→Lys; Gln 28→His; Leu 36→Arg; Ala 40→Asp; Ile 41→Val; Gln 49→Glu; Tyr 52→Glu; Ser 68→Asp; Leu 70→Gly; Arg 72→Val; Lys 73→Gln; Lys 75→Arg; Asp 77→Met; Trp 79→Val; Ile 80→Thr; Arg 81→His; Cys 87→Ser; Lys 98→Glu; Leu 103→Val; Tyr 106→Ala; Asn 114→Asp; Phe 123→Val; Lys 125→Leu; Ser 127→Lys; Tyr 132→Leu; Lys 134→Glu; and, optionally, a Gly residue is added N-terminally;
(rr) Pro 9→His; Gln 28→His; Leu 36→Arg; Ala 40→Asp; Ile 41→Val; Gln 49→Glu; Tyr 52→Glu; Ser 68→Asp; Leu 70→Gly; Arg 72→Val; Lys 73→Gln; Lys 75→Arg; Asp 77→Met; Trp 79→Val; Ile 80→Thr; Arg 81→His; Cys 87→Ser; Lys 98→Glu; Leu 103→Val; Tyr 106→Ala; Asn 114→Asp; Phe 123→Val; Lys 125→Leu; Ser 127→Lys; Tyr 132→Leu; Lys 134→Glu; and, optionally, a Gly residue is added N-terminally;
(ss) Ile 8→Lys; Gln 28→His; Leu 36→Arg; Ala 40→Asp; Ile 41→Val; Gln 49→Glu; Tyr 52→Glu; Asn 65→Gln; Ser 68→Asp; Leu 70→Gly; Arg 72→Val; Lys 73→Gln; Lys 75→Arg; Asp 77→Met; Trp 79→Val; Ile 80→Thr; Arg 81→His; Cys 87→Ser; Lys 98→Glu; Leu 103→Val; Tyr 106→Ala; Asn 114→Asp; Phe 123→Val; Lys 125→Leu; Ser 127→Lys; Tyr 132→Leu; Lys 134→Glu; Gly 178→Asp; and, optionally, a Gly residue is added N-terminally; or
(tt) Pro 9→His; Gln 28→His; Leu 36→Arg; Ala 40→Asp; Ile 41→Val; Gln 49→Glu; Tyr 52→Glu; Asn 65→Gln; Ser 68→Asp; Leu 70→Gly; Arg 72→Val; Lys 73→Gln; Lys 75→Arg; Asp 77→Met; Trp 79→Val; Ile 80→Thr; Arg 81→His; Cys 87→Ser; Lys 98→Glu; Leu 103→Val; Tyr 106→Ala; Asn 114→Asp; Phe 123→Val; Lys 125→Leu; Ser 127→Lys; Tyr 132→Leu; Lys 134→Glu; Gly 178→Asp; and, optionally, a Gly residue is added N-terminally.

A CGRP-specific lipocalin mutein of the disclosure may comprise an amino acid sequence selected from the group consisting of SEQ ID NOs: 6-51 and 206-212, or a fragment or variant thereof. A CGRP-specific lipocalin mutein of the disclosure may have at least 70%, at least 75%, at least 80%, at least 85%, at least 90%, at least 92%, at least 95%, at least 97%, at least 98%, or even higher sequence identity to the amino acid sequences shown in any one of SEQ ID NOs: 6-51 and 206-212.

3. Lipocalin Muteins Specific for Hepcidin

Hepcidin, a peptide hormone typically existing in two forms made of either 20 or 25 amino acids, is expressed and secreted by a number of cells in response to iron loading and inflammation. Hepcidin is produced predominantly in hepatocytes of the liver, plays a central role in the regulation of iron homeostasis, acts as an antimicrobial peptide and is directly or indirectly involved in the development of most iron-deficiency/overload syndromes. A major action of hepcidin is to internalize and degrade the iron exporter ferroportin, which is expressed on all iron-exporting cells. Hepcidin directly binds to ferroportin. A high hepcidin level thus leads to the suppression of intestinal iron absorption and iron release from macrophages and hepatocytes, while a low concentration of hepcidin leads to acceleration of iron release from these cells.

Lipocalin muteins that are specific for hepcidin are disclosed in International patent publications WO 2012/022742 and WO 2013/087654.

A hepcidin-specific lipocalin mutein of the disclosure may be a mutein of hNGAL. As compared to the linear polypeptide sequence of mature hNGAL (SEQ ID NO: 2), such mutein may comprise one of the following sets of amino acid residues at the corresponding sequence positions of mature hNGAL:
(a) Ala 36, Ser 40, Leu 41, Met 49, Asn 70, Gly 72, Gly 73, Ser 77, Leu 79, Leu 125, Val 132;
(b) Leu 36, Arg 40, Val 41, Gln 49, Asp 70, Arg 72, Thr 73, Leu 77, Ser 79, Thr 125, Val 132;
(c) Leu 36, Glu 40, Ile 41, Leu 49, Gln 70, Gly 72, Glu 73, Gly 77, Gly 79, Phe 125, Val 132;
(d) Leu 36, Glu 40, Ile 41, Met 49, Met 70, Leu 72, Ala 73, Glu 77, Leu 79, Val 125, Val 132;
(e) Leu 36, Glu 40, Val 41, Met 49, Met 70, Leu 72, Ala 73, Glu 77, Leu 79, Thr 125, Val 132;
(f) Leu 36, Glu 40, Val 41, Met 49, Met 70, Leu 72, Ala 73, Glu 77, Leu 79, Val 125, Val 132;
(g) Thr 36, Ser 40, Ile 41, Gln 49, Phe 70, Glu 72, Gly 73, Arg 77, Val 79, Val 125, Leu 132;
(h) Val 36, Glu 40, Met 41, Leu 49, Met 70, Glu 72, Tyr 73, Val 77, Leu 79, Arg 125, Val 132;
(i) Val 36, Gly 40, Leu 41, Leu 49, Leu 70, Val 72, Arg 73, Arg 77, Tyr 79, Met 125, Val 132;
(j) Leu 36, Glu 40, Val 41, Met 49, Trp 52, Ile 68, Met 70, Leu 72, Ala 73, Glu 77, Leu 79, Gln 81, Asp 96, Ser 100, Arg 103, Gly 106, Thr 125, Trp 127, Val 132, Trp 134;
(k) Leu 36, Glu 40, Val 41, Met 49, Trp 52, Ile 68, Met 70, Leu 72, Ala 73, Glu 77, Leu 79, Gln 81, Gly 96, Gly 100, Arg 103, Gly 106, Val 125, Trp 127, Val 132, and Trp 134;
(l) Leu 36, Glu 40, Val41, Met 49, Trp 52, Ile 68, Met 70, Leu 72, Ala 73, Glu 77, Leu 79, Gln 81, Asp 96, Ser 100, Arg 103, Gly 106, Val 125, Trp 127, Val 132, Trp 134
(m) Leu 36, Glu 40, Ile 41, Met 49, Trp 52, Ile 68, Met 70, Leu 72, Ala 73, Glu 77, Leu 79, Gln 81, Asp 96, Ser 100, Arg 103, Gly 106, Val 125, Trp 127, Val 132, Trp 134;
(n) Leu 36, Glu 40, Ile 41, Met 49, Trp 52, Ile 68, Met 70, Leu 72, Ala 73, Glu 77, Leu 79, Gln 81, Asp 96, Ser 100, Arg 103, Gly 106, Val 125, Trp 127, Val 132, Trp 134;
(o) Leu 36, Glu 40, Val 41, Met 49, Trp 52, Ile 68, Met 70, Leu 72, Ala 73, Glu 77, Leu 79, Gln 81, Asp 96, Ser 100, Arg 103, Gly 106, Val 125, Trp 127, Val 132, Trp 134;
(p) Leu 36, Glu 40, Val 41, Met 49, Trp 52, Ile 68, Met 70, Leu 72, Ala 73, Glu 77, Leu 79, Gln 81, Gly 96, Gly 100, Arg 103, Gly 106, Val 125, Trp 127, Val 132, Trp 134;
(q) Leu 36, Glu 40, Val 41, Met 49, Trp 52, Ile 68, Met 70, Leu 72, Ala 73, Glu 77, Leu 79, Gln 81, Asp 96, Ser 100, Arg 103, Gly 106, Thr 125, Trp 127, Val 132, Trp 134; or
(r) Leu 36, Glu 40, Val 41, Met 49, Trp 52, Ile 68, Met 70, Leu 72, Ala 73, Glu 77, Leu 79, Gln 81, Asp 96, Ser 100, Arg 103, Gly 106, Val 125, Trp 127, Val 132, Trp 134.

A hepcidin-specific lipocalin mutein of the disclosure may comprise an amino acid sequence selected from the group consisting of SEQ ID NOs: 52-65, or a fragment or variant thereof. A hepcidin-specific lipocalin mutein of the disclosure may have at least 70%, at least 75%, at least 80%, at least 85%, at least 90%, at least 92%, at least 95%, at least 97%, at least 98%, or even higher sequence identity to the amino acid sequences shown in any one of SEQ ID NOs: 52-65.

4. Lipocalin Muteins Specific for PCSK9

Human proprotein convertase subtilisin/kexin type 9 (PCSK9) is a secreted protein expressed primarily in the kidneys, liver and intestines. It has three domains: an inhibitory pro-domain (amino acids 1-152; including a signal sequence at amino acids 1-30), a serine protease domain (or catalytic domain; at amino acids 153-448), and a C-terminal domain (or cysteine/histidine-rich domain) of 210 residues in length (at amino acids 449-692), which is rich in cysteine residues. PCSK9 is synthesized as a zymogen that undergoes autocatalytic cleavage between the pro-domain and catalytic domain in the endoplasmic reticulum. The pro-domain remains bound to the mature protein after cleavage, and the complex is secreted. The cysteine-rich domain may play a role analogous to the P-(processing) domains of other Furin/Kexin/Subtilisin-like serine proteases, which appear to be essential for folding and regulation of the activated protease.

PCSK9 is a member of the proteinase K secretory subtilisin-like subfamily of serine proteases (Naureckiene et al., Arch Biochem Biophys, 2003) and functions as a strong negative regulator of hepatic low-density lipoprotein receptors (LDL-R). PCSK9 plays a critical role in cholesterol metabolism by controlling the levels of low-density lipoprotein (LDL) particles that circulate in the bloodstream. Elevated levels of PCSK9 have been shown to reduce LDL-R levels in the liver, resulting in high levels of low-density lipoprotein cholesterol (LDL-c) in the plasma and increased susceptibility to coronary artery disease (Peterson et al., J Lipid Res, 2008).

Lipocalin muteins that are specific for PCSK9 are disclosed in International patent publications WO 2014/140210.

A PCSK9-specific lipocalin mutein of the disclosure may be a mutein of human tear lipocalin. As compared to the linear polypeptide sequence of mature human tear lipocalin (SEQ ID NO: 1), such mutein may comprise one of the following sets of residues at the corresponding sequence positions of mature human tear lipocalin:
(a) Glu 27→Ser, Phe 28→Arg, Pro 29→Gly, Glu 30→Asp, Met 31→Ala, Leu 33→Trp, Ile 57→Tyr, Asp 80→Met, Glu 104→Pro, Leu 105→Tyr, His 106→Gln, Lys 108→Ala;
(b) Glu 27→Gln, Phe 28→Cys, Pro 29→Asp, Glu 30→Thr, Met 31→Gly, Leu 33→Trp, Ile 57→Tyr, Leu 105→Cys, His 106→Gly, Lys 108→Trp;
(c) Glu 27→Glu, Phe 28→Trp, Pro 29→Asn, Glu 30→Gly, Met 31→His, Leu 33→Tyr, Ile 57→Tyr, Asp 80→Pro, Glu 104→Ser, Leu 105→Trp, His 106→Pro, Lys 108→Tyr;
(d) Glu 27→Thr, Phe 28→Asp, Pro 29→Asn, Glu 30→Ser, Met 31→Pro, Leu 33→Phe, Ile 57→Tyr, Asp 80→Ile, Glu 104→Ala, Leu 105→Glu, His 106→Arg, Lys 108→Arg;
(e) Glu 27→Phe, Phe 28→Lys, Pro 29→Ile, Glu 30→Ala, Met 31→Ser, Leu 33→Pro, Ile 57→Trp, Asp 80→Gln, Glu 104→Asn, Leu 105→Arg, His 106→Gln, Lys 108→Asp;
(f) Glu 27→Lys, Phe 28→Gly, Pro 29→Pro, Glu 30→Thr, Met 31→Pro, Leu 33→Trp, Ile 57→His, Asp 80→Tyr, Glu 104→Ala, Leu 105→Ser, His 106→Val, Lys 108→Asn;
(g) Glu 27→Glu, Phe 28→His, Pro 29→Leu, Glu 30→Ala, Met 31→Asp, Leu 33→Ala, Ile 57→Gln, Asp 80→Ile, Glu 104→Ala, Leu 105→Tyr, His 106→Pro, Lys 108→Ser;
(h) Glu 27→Ala, Phe 28→Asp, Pro 29→Met, Glu 30→Gly, Met 31→Asp, Leu 33→Pro, Ile 57→Thr, Asp 80→Thr, Glu 104→Thr, His 106→Thr, Lys 108→Arg;
(i) Glu 27→Arg, Phe 28→Leu, Pro 29→Asp, Glu 30→Asn, Met 31→Glu, Leu 33→Trp, Ile 57→Tyr, Asp 80→Gln, Glu 104→Pro, Leu 105→Arg, His 106→Asn, Lys 108→Ala;
(j) Glu 27→Lys, Phe 28→Asn, Pro 29→Met, Glu 30→Gly, Met 31→Gln, Leu 33→Pro, Ile 57→Arg, Asp 80→Ile, Glu 104→Asp, Leu 105→Arg, His 106→Leu, Lys 108→Thr;
(k) Glu 27→Ser, Phe 28→Arg, Pro 29→Gly, Glu 30→Asp, Met 31→Ala, Leu 33→Trp, Ile 57→Tyr, Asp 80→Met, Glu 104→Pro, Leu 105→Gly, His 106→Gln, Lys 108→Ala;
(l) Arg 26→Phe, Glu 27→Ser, Phe 28→Arg, Pro 29→Gly, Glu 30→Asp, Met 31→Ala, Asn 32→Ile, Leu 33→Trp, Glu 34→Thr, Leu 56→Met, Ile 57→Tyr, Ser 58→Ala, Lys 83→Ser, Glu 104→Pro and Lys 108→Thr;
(m) Thr 43→Ile or Ala, Glu 45→Gly, Asn 48→Gly, Glu 63→Gly, Ala 66→Val, Glu 69→Val, Lys 70→Arg, Ala 79→Thr, Met or Val, Asp 80→Met or Ser, Gly 82→Ser, His 84→Gln, Val 85→Gly, Tyr 87→Ser, Ile 88→Thr or Leu, His 92→Pro, Leu 105→His, Gly or Tyr and His 106→Gln or Arg;
(n) Glu 27→Phe, Phe 28→Lys, Pro 29→Ile, Asn 32→Trp, Leu 33→Pro, Glu 34→Arg, Leu 56→Asn, Ile 57→Trp, His 106→Gln and Lys 108→Glu; or
(o) Glu 43→Gly or Ala, Glu 45→Gly, Ser 58→Trp or Arg, Glu 63→Asp, Glu 69→Gly, Lys 70→Arg, Asp 80→Gln, Val or Thr, Gly 82→Asp, Lys 83→Ser or Arg, Ala 86→Glu or Ser, Phe 99→Leu, Glu 102→Lys or Val, Glu 104→Asn or Lys and Pro 106→Thr.

A PCSK9-specific lipocalin mutein of the disclosure may comprise an amino acid sequence selected from the group consisting of SEQ ID NOs: 66-91, or a fragment or variant thereof. A PCSK9-specific lipocalin mutein of the disclosure may have at least 70%, at least 75%, at least 80%, at least 85%, at least 90%, at least 92%, at least 95%, at least 97%, at least 98%, or even higher sequence identity to the amino acid sequences shown in any one of SEQ ID NOs: 66-91.

5. Lipocalin Muteins Specific for Pyoverdine and Pyochelin

Pyoverdine (Pvd) is a peptide-linked hydroxamate- and catecholate-type ligand, and pyochelin (Pch) a derivatized conjugate of salicylate and two molecules of cysteine and having phenol, carboxylate, and amine ligand functionalities. Both Pvd and Pch have demonstrated roles in P. aeruginosa virulence with some indication of synergism P. aeruginosa is able to scavenge iron from the host environment by using the secreted iron-binding siderophores, pyochelin and pyoverdine. Double-deficient mutants unable to make either siderophore are much more attenuated in virulence than either single-deficient mutant unable to make just one of the two siderophores (Takase et al., Infect Immun, 2000). Furthermore, pyoverdine acts as a signalling molecule to control production of several virulence factors as well as pyoverdine itself; while it has been proposed that pyochelin may be part of a system for obtaining divalent metals such as ferrous iron and zinc for P. aeruginosa's pathogenicity, in addition to ferric iron (Visca et al., Appl Environ Microbiol, 1992).

Three structurally different pyoverdine types or groups have been identified from several P. aeruginosa strains: from P. aeruginosa ATCC 15692 (G. et al., Liebigs Ann Chem, 1989), from P. aeruginosa ATCC 27853 (Tappe et al., J Prakt Chem, 1993) and from a natural isolate, P. aeruginosa R (Gipp et al., Naturforsch, 1991). Moreover, comparative biological investigations on 88 clinical isolates and the two collection strains mentioned above revealed three different strain-specific pyoverdine-mediated iron uptake systems (Meyer et al., Microbiology, 1997, Cornelis et al., Infect Immun, 1989) according to the reference strains: P. aeruginosa ATCC 15692 (Type I Pvd or Pvd I), P. aeruginosa ATCC 27853 (Type II Pvd or Pvd ii) and the clinical isolates P. aeruginosa R and pa6 (Type III Pvd or Pvd III).

Lipocalin muteins that are specific for Pvd type I, Pvd type II, Pvd type III, and Pch are disclosed in International patent publications WO 2016/131804.

A Pvd type I-specific lipocalin mutein of the disclosure may be a mutein of hNGAL. As compared to the linear polypeptide sequence of mature hNGAL (SEQ ID NO: 2), such mutein may comprise one of the following sets of mutated amino acid residues:
(a) Gln 28→His; Leu 36→Asn; Ala 40→Gly; Ile 41→Trp; Gln 49→Ile; Tyr 52→Met; Ser 68→Val; Leu 70→Gln; Arg 72→Trp; Lys 73→Asp; Asp 77→Leu; Trp 79→Gln; Arg 81→Gln; Cys 87→Ser; Asn 96→His; Tyr 100→Lys; Leu 103→His; Tyr 106→His; Lys 125→Arg; Ser 127→Trp; Tyr 132→Trp; Lys 134→Asp;
(b) Gln 28→His; Leu 36→Thr; Ala 40→Gly; Ile 41→Phe; Gln 49→Leu; Tyr 52→Trp; Leu 70→Trp; Arg 72→Ala; Lys 73→Leu; Asp 77→Tyr; Trp 79→Asp; Arg 81→Gly; Cys 87→Ser; Asn 96→Ile; Tyr 100→Glu; Leu 103→His; Tyr 106→Gln; Lys 125→Trp; Ser 127→Asn; Tyr 132→Asn; Lys 134→Gln;
(c) Gln 28→His; Leu 36→Trp; Ala 40→Thr; Ile 41→Thr; Gln 49→Pro; Tyr 52→Pro; Ser 68→Asp; Leu 70→Gln; Arg 72→Ser; Lys 73→Glu; Asp 77→Ser; Trp 79→Ser; Arg 81→Ile; Cys 87→Ser; Asn 96→Gly; Tyr 100→Asn; Leu 103→Lys; Tyr 106→His; Lys 125→Tyr; Ser 127→Ala; Tyr 132→Gly; Lys 134→Asn;
(d) Gln 28→His; Leu 36→Phe; Ala 40→Asn; Ile 41→Arg; Gln 49→Pro; Tyr 52→Met; Ser 68→Asp; Leu 70→Thr; Arg 72→Glu; Lys 73→Ala; Asp 77→Arg; Trp 79→Arg; Arg 81→Ile; Cys 87→Ser; Asn 96→Tyr; Tyr 100→Lys; Leu 103→Pro; Tyr 106→Phe; Lys 125→Ser; Ser 127→Thr; Tyr 132→Trp; Lys 134→Gly;
(e) Gln 28→His; Ala 40→Gly; Ile 41→Trp; Gln 49→Val; Tyr 52→Met; Ser 68→Val; Leu 70→Asp; Arg 72→Glu; Lys 73→Leu; Asp 77→Arg; Trp 79→Met; Arg 81→Glu; Cys 87→Ser; Asn 96→Asp; Tyr 100→Phe; Leu 103→Trp; Tyr 106→Gln; Lys 125→Gly; Ser 127→Tyr; Tyr 132→Trp; Lys 134→His;
(f) Gln 28→His; Leu 36→Val; Ala 40→Phe; Ile 41→Phe; Gln 49→Ala; Tyr 52→Pro; Ser 68→Glu; Leu 70→Trp; Arg 72→Leu; Lys 73→Asn; Asp 77→Gln; Trp 79→Glu; Arg 81→His; Cys 87→Ser; Asn 96→Tyr; Leu 103→Tyr; Tyr 106→His; Lys 125→Val; Ser 127→His; Tyr 132→Lys; Lys 134→Trp;
(g) Gln 28→His; Leu 36→Trp; Ala 40→Thr; Ile 41→Thr; Gln 49→Pro; Tyr 52→Pro; Ser 68→Asp; Leu 70→Gln; Arg 72→Ser; Lys 73→Glu; Asp 77→Ser; Trp 79→Ser; Ile 80→Thr; Arg 81→Ile; Cys 87→Ser; Asn 96→Gly; Tyr 100→Ser; Leu 103→Gln; Tyr 106→His; Lys 125→Tyr; Ser 127→Ile; Tyr 132→Gly; Lys 134→Asn;
(h) Gln 28→His; Leu 36→Trp; Ala 40→Thr; Ile 41→Thr; Gln 49→Pro; Tyr 52→Pro; Ser 68→Asp; Leu 70→Gln; Arg 72→Ser; Lys 73→Asp; Asp 77→Ser; Trp 79→Ser; Arg 81→Ile; Cys 87→Ser; Asn 96→Gly; Tyr 100→Asn; Leu 103→Asp; Tyr 106→His; Lys 125→Tyr; Ser 127→Val; Tyr 132→Gly; Lys 134→Asn;
(i) Gln 28→His; Leu 36→Trp; Ala 40→Thr; Ile 41→Thr; Gln 49→Pro; Tyr 52→Pro; Ser 68→Asp; Leu 70→Gln; Arg 72→Ser; Lys 73→Glu; Asp 77→Thr; Trp 79→Ser; Arg 81→Ile; Cys 87→Ser; Asn 96→Asp; Tyr 100→Asn; Leu 103→Glu; Tyr 106→His; Lys 125→Tyr; Ser 127→Asp; Tyr 132→Gly; Lys 134→Asn;
(j) Gln 28→His; Leu 36→Trp; Ala 40→Thr; Ile 41→Thr; Gln 49→Pro; Tyr 52→Pro; Ser 68→Asp; Leu 70→Gln; Arg 72→Ser; Lys 73→Asp; Asp 77→Val; Trp 79→Ser; Arg 81→Ile; Cys 87→Ser; Asn 96→Gly; Tyr 100→Asn; Leu 103→Asn; Tyr 106→His; Lys 125→Tyr; Ser 127→Via; Tyr 132→Gly; Lys 134→Asn;
(k) Gln 28→His; Ala 40→Gly; Ile 41→Trp; Gln 49→Leu; Tyr 52→Met; Ser 68→Val; Leu 70→Asp; Arg 72→Glu; Lys 73→Leu; Asp 77→Arg; Trp 79→Met; Arg 81→Glu; Cys 87→Ser; Asn 96→Asp; Tyr 100→Ser; Leu 103→Trp; Tyr 106→Gln; Lys 125→Gly; Ser 127→Tyr; Tyr 132→Trp; Lys 134→His;
(l) Gln 28→His; Leu 36→Trp; Ala 40→Thr; Ile 41→Thr; Gln 49→Pro; Tyr 52→Pro; Thr 54→Val; Ser 68→Asp; Leu 70→Gln; Arg 72→Ser; Lys 73→Glu; Lys 75→Glu; Asp 77→Ser; Trp 79→Ser; Ile 80→Thr; Arg 81→Ile; Cys 87→Ser; Asn 96→Gly; Tyr 100→Ser; Leu 103→Gln; Tyr 106→His; Lys 125→Tyr; Ser 127→Thr; Tyr 132→Gly; Lys 134→Asn;
(m) Gln 28→His; Ala 40→Gly; Ile 41→Trp; Lys 46→Glu; Gln 49→Leu; Tyr 52→Met; Thr 54→Ala; Ile 55→Via; Lys 59→Arg; Ser 68→Val; Leu 70→Asp; Arg 72→Glu; Lys 73→Leu; Lys 74→Glu; Lys 75→Glu; Asp 77→Arg; Trp 79→Met; Ile 80→Thr; Arg 81→Glu; Ser 87→Asn; Asn 96→Asp; Tyr 100→Ser; Leu 103→Trp; Tyr 106→Gln; Lys 125→Gly; Ser 127→Tyr; Tyr 132→Trp; Lys 134→His;
(n) Leu 36→Trp; Asn 39→Asp; Ala 40→Thr; Ile 41→Thr; Gln 49→Pro; Tyr 52→Pro; Thr 54→Val; Asn 65→Asp; Ser 68→Asp; Leu 70→Gln; Arg 72→Ser; Lys 73→Glu; Lys 75→Glu; Asp 77→Ser; Trp 79→Ser; Ile 80→Thr; Arg 81→Ile; Cys 87→Ser; Asn 96→Gly; Tyr 100→Ser; Leu 103→Gln; Tyr 106→His; Lys 125→Tyr; Ser 127→Thr; Tyr 132→Gly; Lys 134→Asn; Thr 136→Ala;
(o) Leu 36→Trp; Ala 40→Thr; Ile 41→Ala; Gln 49→Pro; Tyr 52→Pro; Thr 54→Val; Asn 65→Asp; Ser 68→Asp; Leu 70→Gln; Arg 72→Ser; Lys 73→Glu; Lys 75→Glu; Asp 77→Ser; Trp 79→Ser; Ile 80→Thr; Arg 81→Ile; Cys 87→Ser; Asn 96→Gly; Tyr 100→Ser; Leu 103→Gln; Tyr 106→His; Lys 125→Tyr; Ser 127→Thr; Tyr 132→Gly; Lys 134→Asn; Thr 136→Ala;
(p) Gln 28→His; Ala 40→Gly; Ile 41→Trp; Lys 46→Glu; Gln 49→Leu; Tyr 52→Met; Thr 54→Ala; Ile 55→Via; Lys 59→Arg; Asn 65→Asp; Ser 68→Val; Leu 70→Asp; Arg 72→Glu; Lys 73→Leu; Lys 74→Glu; Lys 75→Glu; Asp 77→Arg; Trp 79→Met; Ile 80→Thr, Arg 81→Glu; Ser 87→Asn; Asn 96→Asp; Tyr 100→sER; Leu 103→Trp; Tyr 106→Gln; Lys 125→Gly; Ser 127→Tyr; Tyr 132→Trp; Lys 134→His; or
(q) Gln 28→His; Ala 40→Gly; Ile 41→Trp; Lys 46→Glu; Gln 49→Leu; Tyr 52→Met; Thr 54→Ala; Ile 55→Val; Lys 59→Arg; Asn 65→Gln; Ser 68→Val; Leu 70→Asp; Arg 72→Glu; Lys 73→Leu; Lys 74→Glu; Lys 75→Glu; Asp 77→Arg; Trp 79→Met; Ile 80→Thr, Arg 81→Glu; Ser 87→Asn; Asn 96→Asp; Tyr 100→Ser; Leu 103→Trp; Tyr 106→Gln; Lys 125→Gly; Ser 127→Tyr; Tyr 132→Trp; Lys 134→His.

A Pvd type II-specific lipocalin mutein of the disclosure may be a mutein of hNGAL. As compared to the linear polypeptide sequence of mature hNGAL (SEQ ID NO: 2), such mutein may comprise one of the following sets of mutated amino acid residues:
(a) Gln 28→His; Leu 36→Val; Ala 40→Glu; Ile 41→Val; Gln 49→Gly; Tyr 52→Pro; Ser 68→Glu; Leu 70→Arg; Arg 72→His; Lys 73→Asn; Asp 77→Asn; Trp 79→Ser; Arg 81→Glu; Cys 87→Ser; Tyr 100→Asn; Leu 103→Gln; Tyr 106→Met; Ser 127→Lys; Tyr 132→Gly; Lys 134→Trp;
(b) Gln 28→His; Ala 40→Thr; Ile 41→Ile; Gln 49→Gly; Tyr 52→Asn; Ser 68→Asp; Leu 70→Arg; Arg 72→Ile; Lys 73→Met; Asp 77→His; Trp 79→Tyr; Arg 81→Glu; Cys 87→Ser; Asn 96→Ile; Tyr 100→Asn; Leu 103→Thr; Tyr 106→Gln; Lys 125→Ile; Ser 127→Arg; Tyr 132→Met; Lys 134→Trp;
(c) Gln 28→His; Leu 36→Ile; Ala 40→Thr; Ile 41→Val; Gln 49→Gly; Tyr 52→Pro; Ser 68→Glu; Leu 70→Arg; Arg 72→Ala; Lys 73→Pro; Asp 77→Ile; Trp 79→Ser; Arg 81→Ser; Cys 87→Ser; Asn 96→Met; Tyr 100→Ser; Leu 103→Gly; Tyr 106→Ala; Lys 125→Lys; Tyr 132→Val; Lys 134→Trp;
(d) Gln 28→His; Ala 40→Asn; Gln 49→Ala; Tyr 52→Pro; Ser 68→Glu; Leu 70→Arg; Arg 72→Ser; Lys 73→Gln; Asp 77→Met; Trp 79→Ala; Arg 81→Tyr; Cys 87→Ser; Asn 96→Arg; Tyr 100→Pro; Leu 103→Thr; Tyr 106→Ile; Lys 125→Lys; Ser 127→Met; Tyr 132→Phe; Lys 134→Trp;
(e) Gln 28→His; Ala 40→His; Gln 49→Ala; Tyr 52→Pro; Ser 68→Glu; Leu 70→Asp; Arg 72→Gly; Lys 73→Arg; Asp 77→His; Trp 79→Trp; Arg 81→Glu; Cys 87→Ser; Asn 96→Arg; Tyr 100→Asp; Leu 103→Met; Tyr 106→Phe; Lys 125→Ala; Ser 127→Asp; Tyr 132→Asn; Lys 134→Trp;
(f) Gln 28→His; Leu 36→Asn; Ala 40→Gly; Ile 41→Arg; Gln 49→Pro; Tyr 52→Trp; Ser 68→Arg; Leu 70→Trp; Arg 72→Asn; Lys 73→Gln; Asp 77→Lys; Trp 79→Asp; Arg 81→Glu; Cys 87→Ser; Asn 96→Asp; Tyr 100→Thr; Leu 103→Trp; Tyr 106→Asn; Lys 125→Asn; Ser 127→Met; Tyr 132→Ile; Lys 134→Tyr;
(g) Gln 28→His; Leu 36→Val; Ala 40→Thr; Ile 41→Thr; Gln 49→Gly; Tyr 52→Gly; Ser 68→Glu; Leu 70→Arg; Arg 72→Gly; Lys 73→Arg; Asp 77→Gly; Trp 79→Trp; Arg 81→Glu; Cys 87→Ser; Asn 96→Ala; Tyr 100→Trp; Leu 103→Ile; Tyr 106→Gly; Lys 125→Lys; Ser 127→Asn; Tyr 132→Val; Lys 134→Trp;
(h) Gln 28→His; Leu 36→Val; Ala 40→Glu; Ile 41→Val; Gln 49→Gly; Tyr 52→Pro; Ser 68→Glu; Leu 70→Arg; Arg 72→His; Lys 73→Asn; Asp 77→Asn; Trp 79→Ser; Arg 81→Glu; Cys 87→Ser; Asn 96→Lys; Tyr 100→Asn; Leu 103→Val; Tyr 106→Met; Lys 125→Asn; Ser 127→Lys; Tyr 132→Gly; Lys 134→Trp;
(i) Gln 28→His; Leu 36→Val; Ala 40→Thr; Ile 41→Val; Gln 49→Gly; Tyr 52→Pro; Ser 68→Glu; Leu 70→Arg; Arg 72→His; Lys 73→Asn; Asp 77→Asn; Trp 79→Ser; Arg 81→Glu; Cys 87→Ser; Leu 103→Gln; Tyr 106→Met; Ser 127→Lys; Tyr 132→Val; Lys 134→Trp;
(j) Gln 28→His; Leu 36→Val; Ala 40→Thr; Ile 41→Val; Gln 49→Gly; Tyr 52→Pro; Ser 68→Glu; Leu 70→Arg; Arg 72→His; Asp 77→Asn; Trp 79→Phe; Arg 81→Glu; Cys 87→Ser; Asn 96→Lys; Tyr 100→His; Leu 103→Gln; Tyr 106→Met; Ser 127→Lys; Tyr 132→Ala; Lys 134→Trp;
(k) Gln 28→His; Leu 36→Val; Ala 40→Gly; Ile 41→Val; Gln 49→Gly; Tyr 52→Pro; Ser 68→Glu; Leu 70→Arg; Arg 72→His; Lys 73→Asn; Asp 77→Asn; Trp 79→Trp; Arg 81→Glu; Cys 87→Ser; Tyr 100→Asn; Leu 103→His; Tyr 106→Met; Ser 127→Lys; Tyr 132→Gly; Lys 134→Trp;
(l) Gln 28→His; Leu 36→Val; Ala 40→Thr; Ile 41→Ile; Gln 49→Gly; Tyr 52→Asn; Ser 68→Asp; Leu 70→Arg; Arg 72→Ile; Lys 73→Phe; Asp 77→His; Trp 79→Tyr; Arg 81→Asp; Cys 87→Ser; Leu 103→Met; Tyr 106→Gln; Lys 125→Ile; Ser 127→Arg; Tyr 132→Ile; Lys 134→Trp;
(m) Gln 28→His; Leu 36→Val; Ala 40→Thr; Ile 41→Ile; Gln 49→Gly; Tyr 52→Asn; Ser 68→Asp; Leu 70→Arg; Arg 72→Ile; Lys 73→Arg; Asp 77→His; Trp 79→Tyr; Arg 81→Asp; Cys 87→Ser; Leu 103→Thr; Tyr 106→Gln; Lys 125→Ile; Ser 127→Arg; Tyr 132→Ile; Lys 134→Trp;
(n) Gln 28→His; Leu 36→Val; Ala 40→Glu; Ile 41→Val; Gln 49→Gly; Tyr 52→Pro; Asn 65→Asp; Ser 68→Glu; Leu 70→Arg; Arg 72→His; Lys 73→Asn; Asp 77→Asn; Trp 79→Phe; Arg 81→Glu; Cys 87→Ser; Asn 96→Lys; Tyr 100→Asn; Leu 103→Val; Tyr 106→Met; Lys 125→Asn; Ser 127→Lys; Tyr 132→Gly; Lys 134→Trp;
(o) Gln 28→His; Leu 36→Val; Ala 40→Glu; Ile 41→Val; Gln 49→Gly; Tyr 52→Pro; Asn 65→Gln; Ser 68→Glu; Leu 70→Arg; Arg 72→His; Lys 73→Asn; Asp 77→Asn; Trp 79→Phe; Arg 81→Glu; Cys 87→Ser; Asn 96→Lys; Tyr 100→Asn; Leu 103→Val; Tyr 106→Met; Lys 125→Asn; Ser 127→Lys; Tyr 132→Gly; Lys 134→Trp;
(p) Gln 28→His; Leu 36→Val; Ala 40→Thr; Ile 41→Ile; Gln 49→Gly; Tyr 52→Asn; Thr 54→Ala; Asn 65→Asp; Ser 68→Asp; Leu 70→Arg; Arg 72→Ile; Lys 73→Arg; Asp 77→His; Trp 79→Tyr; Arg 81→Asp; Cys 87→Ser; Leu 103→Thr; Tyr 106→Gln; Lys 125→Ile; Ser 127→Arg; Tyr 132→Ile; Lys 134→Trp;
(q) Gln 28→His; Leu 36→Val; Ala 40→Thr; Ile 41→Ile; Gln 49→Gly; Tyr 52→Asn; Thr 54→Ala; Asn 65→Gln; Ser 68→Asp; Leu 70→Arg; Arg 72→Ile; Lys 73→Arg; Asp 77→His; Trp 79→Tyr; Arg 81→Asp; Cys 87→Ser; Leu 103→Thr; Tyr 106→Gln; Lys 125→Ile; Ser 127→Arg; Tyr 132→Ile; Lys 134→Trp;
(r) Leu 36→Val; Ala 40→Thr; Ile 41→Ile; Gln 49→Gly; Tyr 52→Asn; Thr 54→Ala; Asn 65→Asp; Ser 68→Asp; Leu 70→Arg; Arg 72→Ile; Lys 73→Arg; Asp 77→His; Trp 79→Tyr; Arg 81→Asp; Cys 87→Ser; Leu 103→Thr; Tyr 106→Gln; Lys 125→Ile; Ser 127→Arg; Tyr 132→Ile; Lys 134→Trp; or
(s) Gln 28→His; Leu 36→Val; Ala 40→Thr; Ile 41→Ile; Gln 49→Gly; Tyr 52→Asn; Asn 65→Gln; Ser 68→Asp; Leu 70→Arg; Arg 72→Ile; Lys 73→Arg; Asp 77→His; Trp 79→Tyr; Arg 81→Asp; Cys 87→Ser; Leu 103→Thr; Tyr 106→Gln; Lys 125→Ile; Ser 127→Arg; Tyr 132→Ile; Lys 134→Trp.

A Pvd type III-specific lipocalin mutein of the disclosure may be a mutein of hNGAL. As compared to the linear polypeptide sequence of mature hNGAL (SEQ ID NO: 2), such mutein may comprise one of the following sets of mutated amino acid residues:
(a) Gln 28→His; Leu 36→Phe; Ala 40→Trp; Ile 41→Met; Gln 49→His; Tyr 52→Asn; Ser 68→Glu; Leu 70→Lys; Arg 72→Gln; Lys 73→Ala; Asp 77→Ile; Trp 79→Ser; Arg 81→His; Cys 87→Ser; Asn 96→Ile; Tyr 100→Asn; Leu 103→Gly; Tyr 106→Glu; Lys 125→Trp; Ser 127→His; Tyr 132→Phe; Lys 134→Gln;
(b) Gln 28→His; Leu 36→Phe; Ala 40→Arg; Ile 41→Trp; Gln 49→Ile; Tyr 52→Tyr; Ser 68→Gln; Leu 70→Asn; Arg 72→Trp; Lys 73→Leu; Asp 77→Ala; Trp 79→Ser; Arg 81→Ser; Cys 87→Ser; Asn 96→Arg; Tyr 100→Ile; Leu 103→Pro; Tyr 106→Glu; Lys 125→Thr; Ser 127→Ile; Tyr 132→Phe; Lys 134→Glu;
(c) Gln 28→His; Leu 36→Phe; Ala 40→Leu; Ile 41→Leu; Gln 49→Arg; Tyr 52→Arg; Ser 68→Asp; Leu 70→Arg; Arg 72→Leu; Lys 73→Tyr; Asp 77→Ile; Trp 79→Ser; Arg 81→Ala; Cys 87→Ser; Asn 96→Gly; Tyr 100→Ala; Leu 103→Phe; Tyr 106→Glu; Lys 125→Trp; Ser 127→Ala; Lys 134→Glu;
(d) Gln 28→His; Leu 36→Phe; Ala 40→Trp; Ile 41→Arg; Gln 49→Pro; Tyr 52→Ser; Ser 68→Asn; Leu 70→Arg; Arg 72→Trp; Lys 73→Pro; Asp 77→Arg; Trp 79→Ser; Arg 81→Ser; Cys 87→Ser; Asn 96→Met; Tyr 100→Pro; Leu 103→Gly; Tyr 106→Glu; Lys 125→Trp; Ser 127→Phe; Tyr 132→Phe; Lys 134→Glu;
(e) Gln 28→His; Leu 36→Phe; Ala 40→Trp; Ile 41→Arg; Gln 49→Pro; Tyr 52→Ser; Ser 68→Asn; Leu 70→Arg; Arg 72→Trp; Lys 73→Pro; Asp 77→Arg; Trp 79→Ser; Arg 81→Ser; Cys 87→Ser; Asn 96→Met; Tyr 100→Pro; Leu 103→Gly; Tyr 106→Glu; Lys 125→Trp; Ser 127→Phe; Tyr 132→Phe; Lys 134→Glu;
(f) Gln 28→His; Leu 36→Glu; Ala 40→Leu; Ile 41→Ala; Gln 49→Lys; Tyr 52→Met; Ser 68→Glu; Leu 70→Arg; Lys 73→His; Asp 77→Gln; Trp 79→Asp; Arg 81→Ala; Cys 87→Ser; Asn 96→Leu; Tyr 100→Asp; Leu 103→Gln; Tyr 106→Glu; Ser 127→Val; Tyr 132→Phe; Lys 134→Trp;
(g) Gln 28→His; Leu 36→Glu; Ala 40→Leu; Ile 41→Ala; Gln 49→Met; Tyr 52→Met; Ser 68→Glu; Leu 70→Arg; Lys 73→Gln; Asp 77→Lys; Trp 79→Asp; Arg 81→Val; Cys 87→Ser; Asn 96→Leu; Tyr 100→Asp; Leu 103→Gln; Tyr 106→Glu; Ser 127→Val; Tyr 132→Phe; Lys 134→Trp;
(h) Gln 28→His; Leu 36→Glu; Ala 40→Leu; Ile 41→Thr; Gln 49→Met; Tyr 52→Met; Ser 68→Glu; Leu 70→Arg; Lys 73→Arg; Asp 77→Lys; Trp 79→Asp; Arg 81→Val; Cys 87→Ser; Asn 96→Val; Tyr 100→Asp; Leu 103→Gln; Tyr 106→Glu; Ser 127→Val; Tyr 132→Phe; Lys 134→Trp;
(i) Gln 28→His; Leu 36→Glu; Ala 40→Leu; Ile 41→Ala; Gln 49→Met; Tyr 52→Met; Ser 68→Glu; Leu 70→Arg; Lys 73→His; Asp 77→Lys; Trp 79→Asp; Arg 81→Val; Cys 87→Ser; Asn 96→Leu; Tyr 100→Asp; Leu 103→Gln; Tyr 106→Glu; Ser 127→Val; Tyr 132→Phe; Lys 134→Trp;
(j) Gln 28→His; Leu 36→Glu; Ala 40→Leu; Ile 41→Ala; Gln 49→Lys; Tyr 52→Met; Ser 68→Glu; Leu 70→Arg; Lys 73→Tyr; Asp 77→Gln; Trp 79→Asp; Arg 81→Val; Cys 87→Ser; Asn 96→-; Tyr 100→Glu; Leu 103→Gln; Tyr 106→Glu; Ser 127→Val; Tyr 132→Phe; Lys 134→Trp;
(k) Gln 28→His; Leu 36→Glu; Ala 40→Leu; Ile 41→Ala; Leu 42→Arg; Gln 49→Met; Tyr 52→Met; Ser 68→Glu; Leu 70→Arg; Lys 73→His; Asp 77→Lys; Trp 79→Asp; Arg 81→Val; Cys 87→Ser; Asn 96→Leu; Tyr 100→Asp; Leu 103→Gln; Ser 105→Pro; Tyr 106→Glu; Ser 127→Val; Tyr 132→Phe; Lys 134→Trp;
(l) Gln 28→His; Leu 36→Glu; Ala 40→Leu; Ile 41→Ala; Asp 47→Asn; Gln 49→Met; Tyr 52→Met; Ser 68→Glu; Leu 70→Arg; Lys 73→His; Asp 77→Lys; Trp 79→Asp; Arg 81→Val; Cys 87→Ser; Asn 96→Leu; Tyr 100→Asp; Leu 103→Gln; Ser 105→Pro; Tyr 106→Glu; Ser 127→Val; Tyr 132→Phe; Lys 134→Trp; Thr 145→Pro;
(m) Gln 28→His; Leu 36→Glu; Ala 40→Leu; Ile 41→Ala; Asp 45→Gly; Lys 46→Arg; Gln 49→Met; Tyr 52→Met; Ser 68→Glu; Leu 70→Arg; Lys 73→His; Asp 77→Lys; Trp 79→Asp; Arg 81→Val; Cys 87→Ser; Asn 96→Leu; Tyr 100→Asp; Leu 103→Gln; Ser 105→Pro; Tyr 106→Glu; Ser 127→Val; Tyr 132→Phe; Lys 134→Trp;
(n) Gln 28→His; Leu 36→Glu; Ala 40→Leu; Ile 41→Ala; Leu 42→Arg; Gln 49→Met; Tyr 52→Met; Asn 65→Asp; Ser 68→Glu; Leu 70→Arg; Lys 73→His; Asp 77→Lys; Trp 79→Asp; Arg 81→Val; Cys 87→Ser; Asn 96→Leu; Tyr 100→Asp; Leu 103→Gln; Ser 105→Pro; Tyr 106→Glu; Ser 127→Val; Tyr 132→Phe; Lys 134→Trp;
(o) Gln 28→His; Leu 36→Glu; Ala 40→Leu; Ile 41→Ala; Asp 47→Asn; Gln 49→Met; Tyr 52→Met; Asn 65→Asp; Ser 68→Glu; Leu 70→Arg; Lys 73→His; Asp 77→Lys; Trp 79→Asp; Arg 81→Val; Cys 87→Ser; Asn 96→Leu; Tyr 100→Asp; Leu 103→Gln; Ser 105→Pro; Tyr 106→Glu; Ser 127→Val; Tyr 132→Phe; Lys 134→Trp; Thr 145→Pro;
(p) Gln 28→His; Leu 36→Glu; Ala 40→Leu; Ile 41→Ala; Asp 45→Gly; Lys 46→Arg; Gln 49→Met; Tyr 52→Met; Asn 65→Asp; Ser 68→Glu; Leu 70→Arg; Lys 73→His; Asp 77→Lys; Trp 79→Asp; Arg 81→Val; Cys 87→Ser; Asn 96→Leu; Tyr 100→Asp; Leu 103→Gln; Ser 105→Pro; Tyr 106→Glu; Ser 127→Val; Tyr 132→Phe; Lys 134→Trp; or
(q) Leu 36→Glu; Ala 40→Leu; Ile 41→Ala; Leu 42→Arg; Gln 49→Met; Tyr 52→Met; Asn 65→Asp; Ser 68→Glu; Leu 70→Arg; Lys 73→His; Asp 77→Lys; Trp 79→Asp; Arg 81→Val; Cys 87→Ser; Asn 96→Leu; Tyr 100→Asp; Leu 103→Gln; Ser 105→Pro; Tyr 106→Glu; Ser 127→Val; Tyr 132→Phe; Lys 134→Trp.

A Pch-specific lipocalin mutein of the disclosure may be a mutein of hNGAL. As compared to the linear polypeptide sequence of mature hNGAL (SEQ ID NO: 2), such mutein may comprise one of the following sets of mutated amino acid residues:
(a) Gln 28→His; Ala 40→Ile; Ile 41→Leu; Gln 49→His; Tyr 52→Leu; Ser 68→His; Leu 70→Thr; Arg 72→Lys; Lys 73→Trp; Asp 77→Ile; Trp 79→Ser; Arg 81→His; Cys 87→Ser; Asn 96→Met; Tyr 100→Asn; Leu 103→His; Tyr 106→Met; Lys 125→Trp; Ser 127→Asp; Tyr 132→Glu; Lys 134→Leu
(b) Gln 28→His; Leu 36→His; Ala 40→Gln; Ile 41→Trp; Gln 49→Arg; Tyr 52→Trp; Ser 68→Asp; Leu 70→Asp; Arg 72→Ala; Lys 73→Ile; Asp 77→His; Trp 79→Arg; Arg 81→Thr; Cys 87→Ser; Tyr 100→His; Leu 103→Gly; Tyr 106→Gly; Lys 125→Phe; Ser 127→Ile; Tyr 132→Ala; Lys 134→Phe;
(c) Gln 28→His; Leu 36→Met; Ala 40→Phe; Ile 41→His; Gln 49→Ser; Tyr 52→Pro; Ser 68→His; Leu 70→Pro; Arg 72→Trp; Lys 73→Ala; Asp 77→Ala; Trp 79→Lys; Arg 81→Ile; Cys 87→Ser; Asn 96→Ala; Tyr 100→Gly; Leu 103→Met; Tyr 106→Trp; Lys 125→Gly; Ser 127→Trp; Tyr 132→Thru; Lys 134→Val;
(d) Gln 28→His; Leu 36→Val; Ala 40→Tyr; Ile 41→Trp; Gln 49→Ala; Ser 68→Asp; Leu 70→Arg; Arg 72→Trp; Lys 73→Arg; Asp 77→Arg; Trp 79→Asp; Arg 81→Trp; Cys 87→Ser; Asn 96→Pro; Tyr 100→Glu; Leu 103→Gln; Tyr 106→Arg; Lys 125→Leu; Ser 127→Arg; Tyr 132→Ala; Lys 134→Asn;
(e) Gln 28→His; Val 34→Leu; Leu 36→Met; Ala 40→Phe; Ile 41→His; Gln 49→Ser; Tyr 52→Pro; Ser 68→His; Leu 70→Pro; Arg 72→Trp; Lys 73→Ala; Asp 77→Ala; Trp 79→Lys; Ile 80→Thr; Arg 81→Ile; Cys 87→Ser; Asn 96→Ala; Tyr 100→Gly; Leu 103→Met; Tyr 106→Trp; Phe 123→Ser; Lys 125→Gly; Ser 127→Trp; Tyr 132→Thru; Lys 134→Val; Thr 141→Ala;
(f) Gln 28→His; Leu 36→Met; Ala 40→Phe; Ile 41→His; Gln 49→Ser; Tyr 52→Pro; Ser 68→His; Leu 70→Pro; Arg 72→Trp; Lys 73→Ala; Asp 77→Ala; Trp 79→Lys; Ile 80→Thr; Arg 81→Ile; Cys 87→Ser; Asn 96→Ala; Tyr 100→Gly; Leu 103→Met; Tyr 106→Trp; Phe 123→Ser; Lys 125→Gly; Ser 127→Trp; Tyr 132→Thru; Lys 134→Val;
(g) Gln 28→His; Leu 36→His; Ala 40→Gln; Ile 41→Trp; Asp 45→Gly; Lys 46→Arg; Gln 49→Arg; Tyr 52→Trp; Ser 68→Asp; Leu 70→Asp; Arg 72→Ala; Lys 73>Ile; Asp 77→Leu; Trp 79→Arg; Arg 81→Thr; Cys 87→Ser; Tyr 100→His; Leu 103→Gly; Tyr 106→Gly; Lys 125→Phe; Ser 127→Ile; Tyr 132→Ala; Lys 134→Phe;
(h) Gln 28→His; Leu 36→His; Ala 40→Gln; Ile 41→Trp; Glu 44→Gly; Lys 46→Tyr; Gln 49→Arg; Tyr 52→Trp; Ser 68→Asp; Leu 70→Asp; Arg 72→Ala; Lys 73→Ile; Lys 74→Glu; Asp 77→His; Trp 79→Arg; Arg 81→Thr; Cys 87→Ser; Leu 94→Phe; Tyr 100→His; Leu 103→Gly; Tyr 106→Gly; Val 108→Ala; Lys 125→Phe; Ser 127→Ile; Tyr 132→Ala; Lys 134→Phe; or
(i) Leu 36→His; Ala 40→Gln; Ile 41→Trp; Asp 45→Gly; Lys 46→Arg; Gln 49→Arg; Tyr 52→Trp; Asn 65→Asp; Ser 68→Asp; Leu 70→Asp; Arg 72→Ala; Lys 73→Ile; Asp 77→Leu; Trp 79→Arg; Arg 81→Thr; Cys 87→Ser; Tyr 100→His; Leu 103→Gly; Tyr 106→Gly; Lys 125→Phe; Ser 127→Ile; Tyr 132→Ala; Lys 134→Phe

A Pvd type I-specific lipocalin mutein of the disclosure may comprise an amino acid sequence selected from the group consisting of SEQ ID NOs: 115-131, or a fragment or variant thereof. A Pvd type I-specific lipocalin mutein of the disclosure may have at least 70%, at least 75%, at least 80%, at least 85%, at least 90%, at least 92%, at least 95%, at least 97%, at least 98%, or even higher sequence identity to the amino acid sequences shown in any one of SEQ ID NOs: 115-131. A Pvd type II-specific lipocalin mutein of the disclosure may comprise an amino acid sequence selected from the group consisting of SEQ ID NOs: 132-150, or a fragment or variant thereof. A Pvd type II-specific lipocalin mutein of the disclosure may have at least 70%, at least 75%, at least 80%, at least 85%, at least 90%, at least 92%, at least 95%, at least 97%, at least 98%, or even higher sequence identity to the amino acid sequences shown in any one of SEQ ID NOs: 132-150. A Pvd type III-specific lipocalin mutein of the disclosure may comprise an amino acid sequence selected from the group consisting of SEQ ID NOs: 151-166, or a fragment or variant thereof. A Pvd type III-specific lipocalin mutein of the disclosure may have at least 70%, at least 75%, at least 80%, at least 85%, at least 90%, at least 92%, at least 95%, at least 97%, at least 98%, or even higher sequence identity to the amino acid sequences shown in any one of SEQ ID NOs: 151-166. A Pch-specific lipocalin mutein of the disclosure may comprise an amino acid sequence selected from the group consisting of SEQ ID NOs: 167-176, or a fragment or variant thereof. A Pch-specific lipocalin mutein of the disclosure may have at least 70%, at least 75%, at least 80%, at least 85%, at least 90%, at least 92%, at least 95%, at least 97%, at least 98%, or even higher sequence identity to the amino acid sequences shown in any one of SEQ ID NOs: 167-176.

6. Lipocalin Muteins Specific for Further Targets

Further lipocalin muteins that are specific for therapeutic targets have been described in the art. WO 2011/069992 describes lipocalin mutein that are specific for amyloid beta and extra-domain B of fibronectin. Amyloid beta (Aβ) are peptides that are crucially involved in Alzheimer's disease as the main component of the amyloid plaques found in the brains of Alzheimer patients. The peptides derive from the amyloid precursor protein (APP), which is cleaved by beta secretase and gamma secretase to yield A. Fibronectin (FN) is a large, modular, dimeric glycoprotein comprising multiple domains of type I, II, and III. Alternative splice variants of FN such as the isoform containing the extra-domain B (ED-B), which is incorporated between the FN1117 and FN1118 domains, are expressed in a tissue-specific and developmental stage-dependent manner (Zardi et al., EMBO J, 1987). ED-B is absent from normal adult tissue except during wound healing and neoplastic vascularization. Consequently, ED-B containing fibronectin is abundantly expressed in many different tumor types that attract neovascularization and undergo aberrant angiogenesis. An amyloid beta-specific lipocalin mutein of the disclosure may comprise an amino acid sequence selected from the group consisting of SEQ ID NOs: 195-199, or a fragment or variant thereof. An amyloid beta-specific lipocalin mutein of the disclosure may have at least 70%, at least 75%, at least 80%, at least 85%, at least 90%, at least 92%, at least 95%, at least 97%, at least 98%, or even higher sequence identity to the amino acid sequences shown in any one of SEQ ID NOs: 195-199. A lipocalin mutein specific for fibronectin ED-B of the disclosure may comprise an amino acid sequence selected from the group consisting of SEQ ID NOs: 200-203, or a fragment or variant thereof. A fibronectin ED-B-specific lipocalin mutein of the disclosure may have at least 70%, at least 75%, at least 80%, at least 85%, at least 90%, at least 92%, at least 95%, at least 97%, at least 98%, or even higher sequence identity to the amino acid sequences shown in any one of SEQ ID NOs: 200-203.

WO 2014/076321 and WO 2015/177175 describe lipocalin muteins that are specific for interleukin-17A (IL-17A or IL-17) and interleukin-23 (IL-23), in particular the p19 subunit of interleukin-23 (IL-23p19).

Human IL-17A (CTLA-8, further named as IL-17, Swiss Prot Q16552) is a glycoprotein with a Mr of 17,000 daltons (Spriggs, J Clin Immunol, 1997). IL-17A may exist as either a homodimer IL-17 A/A or as a heterodimer complexed with the homolog IL-17F to form heterodimeric IL-17 A/F. IL-17F (IL-24, ML-1) shares a 55% amino acid identity with IL-17A. IL-17A and IL-17F also share the same receptor (IL-17RA), which is expressed on a wide variety of cells including vascular endothelial cells, peripheral T cells, B cells, fibroblast, lung cells, myelomonocytic cells, and marrow stromal cells (Moseley et al., Cytokine Growth Factor Rev, 2003, Kawaguchi et al., J Allergy Clin Immunol, 2004, Kolls and Linden, Immunity, 2004). IL-17A is mainly expressed by Th17 cells and is present at elevated levels in synovial fluid of patients with rheumatoid arthritis (RA) and has been shown to be involved in early RA development. IL-17A is also over-expressed in the cerebrospinal fluid of multiple sclerosis (MS) patients. In addition, IL-17 is an inducer of TNF-α and IL-1, the latter being mainly responsible for bone erosion and the very painful consequences for affected patients (Lubberts, Cytokine, 2008). Furthermore, inappropriate or excessive production of IL-17A is associated with the pathology of various other diseases and disorders, such as osteoarthritis, loosening of bone implants, acute transplant rejection (Van Kooten et al., J Am Soc Nephrol, 1998, Antonysamy et al., J Immunol, 1999), septicemia, septic or endotoxic shock, allergies, asthma (Molet et al., J Allergy Clin Immunol, 2001), bone loss, psoriasis (Teunissen et al., J Invest Dermatol, 1998), ischemia, systemic sclerosis (Kurasawa et al., Arthritis Rheum, 2000), stroke, and other inflammatory disorders. A lipocalin mutein specific for IL-17A of the disclosure may comprise an amino acid sequence selected from the group consisting of SEQ ID NOs: 99-104, or a fragment or variant thereof. An IL-17A specific lipocalin mutein of the disclosure may have at least 70%, at least 75%, at least 80%, at least 85%, at least 90%, at least 92%, at least 95%, at least 97%, at least 98%, or even higher sequence identity to the amino acid sequences shown in any one of SEQ ID NOs: 99-104.

Interleukin-23 (also known as IL-23) is a heterodimeric cytokine comprised of two subunits, i.e., p19 and p40 (Oppmann et al., Immunity, 2000). The p19 (Swiss Prot Q9NPF7, herein referred to interchangeably as “IL-23p19”) subunit is structurally related to IL-6, granulocyte-colony stimulating factor (G-CSF), and the p35 subunit of IL-12. IL-23 mediates signaling by binding to a heterodimeric receptor, comprised of IL-23R and IL-12beta1. The IL-12beta1 subunit is shared by the IL-12 receptor, which is composed of IL-12beta1 and IL-12beta2. Transgenic p19 mice have been recently described to display profound systemic inflammation and neutrophilia (Wiekowski et al., J Immunol, 2001). Human IL-23 has been reported to promote the proliferation of T cells, in particular memory T cells and can contribute to the differentiation and/or maintenance of Thl 7 cells (Frucht, Sci STKE, 2002). A lipocalin mutein specific for IL-23p19 of the disclosure may comprise an amino acid sequence selected from the group consisting of SEQ ID NOs: 105-114, or a fragment or variant thereof. An IL-23p19 specific lipocalin mutein of the disclosure may have at least 70%, at least 75%, at least 80%, at least 85%, at least 90%, at least 92%, at least 95%, at least 97%, at least 98%, or even higher sequence identity to the amino acid sequences shown in any one of SEQ ID NOs: 105-114.

Cytotoxic T-lymphocyte-associated protein 4 (CTLA-4), is a protein receptor that functions as an immune checkpoint and downregulates immune responses. CTLA-4 is constitutively expressed in regulatory T cells but only upregulated in conventional T cells after activation. CTLA-4 blockade is considered as a means of inhibiting immune system tolerance to tumours and thereby providing a potentially useful immunotherapy strategy for patients with cancer. Lipocain muteins specific for CTLA-4 are disclosed in WO 2006/056464 and WO 2012/072806. A lipocalin mutein specific for CTLA-4 of the disclosure may comprise an amino acid sequence selected from the group consisting of SEQ ID NOs: 92-98, or a fragment or variant thereof. A CTLA-4 specific lipocalin mutein of the disclosure may have at least 70%, at least 75%, at least 80%, at least 85%, at least 90%, at least 92%, at least 95%, at least 97%, at least 98%, or even higher sequence identity to the amino acid sequences shown in any one of SEQ ID NOs: 92-98.

Further lipocalin muteins that are specific for therapeutic targets are for example disclosed in WO 2009/095447, which discloses lipocalin muteins specific for c-Met; WO 2012/065978, WO 2013/174783 and WO 2016/184875, which disclose lipocalin muteins specific for glypican-3; WO 2017/009456 and WO 2018/134274, which disclose lipocalin muteins specific for Lag-3; WO 2016/177762 and WO 2018/087108, which disclose lipocalin mutein specific for CD137; WO 2016/120307, which discloses lipocalin muteins specific for Ang-2; and WO 2008/015239, which discloses lipocalin muteins specific for VEGF.

B. Administration of Lipocalin Muteins of the Disclosure

A lipocalin mutein of the disclosure may be administered by inhalation. Means and devices for inhaled administration of a substance are known to the skilled person and are for example disclosed in WO 94/017784A and Elphick et al. (2015). Such means and devices include nebulizers, metered dose inhalers, powder inhalers, and nasal sprays. Other means and devices suitable for directing inhaled administration of a lipocalin mutein are also known in the art. Nebulizers are useful in producing aerosols from solutions, while metered dose inhalers, dry powder inhalers, etc. are effective in generating small particle aerosols.

A nebulizer is a drug delivery device used to administer medication in the form of a mist inhaled into the lungs. Different types of nebulizers are known to the skilled person and include jet nebulizers, ultrasonic wave nebulizers, vibrating mesh technology, and soft mist inhalers. Some nebulizers provide a continuous flow of nebulized solution, i.e. they will provide continuous nebulization over a long period of time, regardless of whether the subject inhales from it or not, while others are breath-actuated, i.e. the subject only gets some dose when they inhale from it.

A metered-dose inhaler (MDI) is a device that delivers a specific amount of medication to the lungs, in the form of a short burst of liquid aerosolized medicine. Such a metered-dose inhaler commonly consists of three major components; a canister which comprises the formulation to be administered, a metering valve, which allows a metered quantity of the formulation to be dispensed with each actuation, and an actuator (or mouthpiece) which allows the patient to operate the device and directs the liquid aerosol into the patient's lungs.

A dry-powder inhaler (DPI) is a device that delivers medication to the lungs in the form of a dry powder. Dry powder inhalers are an alternative to the aerosol-based inhalers, such as metered-dose inhalers. The medication is commonly held either in a capsule for manual loading or a proprietary blister pack located inside the inhaler.

Nasal sprays can be used for nasal administration, by which a drug is insufflated through the nose. Nasal sprays may provide extremely quick absorption of the medication.

The lipocalin mutein may be administered once, twice, three times, four times, five times, once a week, twice a week, three times a week, four times a week, five times a week, six times a week, once a day, or twice a day.

Inhaled administration of a lipocalin mutein may result in local exposure, systemic exposure, or both local and systemic exposure to the lipocalin mutein. It is believed that the dose of the lipocalin mutein has a strong influence on whether the administration results in local or systemic exposure. In general, it is believed that low doses of the lipocalin mutein tend to result in local exposure while high doses tend to result in systemic exposure.

In some embodiments, local exposure means that about 0.15% or less, 0.1% or less, 0.05% or less, 0.03% or less, 0.02% or less, or 0.01% or less of the delivered dose of the lipocalin mutein enters the circulatory system. In some embodiments, local exposure means that no systemic exposure of the lipocalin mutein is detectable. Systemic exposure of the lipocalin mutein is preferably measured in blood, preferably in blood plasma or blood serum.

In some embodiments, local exposure means that about 20% or more, about 30% or more, about 40% or more, about 50% or more, about 60% or more, about 70% or more, about 80% or more, or about 90% or more of the delivered lipocalin mutein remain in the respiratory tract or the lung, such as in the lung tissue or the epithelial lining fluid.

In order to achieve local exposure, the delivered dose of the lipocalin mutein may be about 0.05 mg to about 1000 mg per administration, preferably 0.05 mg to about 5 mg per administration, preferably about 0.1 mg to about 5 mg per administration, preferably about 0.1 mg to about 2 mg per administration. The delivered dose of the lipocalin mutein may be about 0.05 μg to about 15 mg per kg body weight per administration, preferably about 0.05 μg to about 100 μg per kg body weight per administration, preferably about 0.05 μg to about 50 μg per kg body weight per administration, preferably about 0.1 μg to about 50 μg per kg body weight per administration. In general, the delivered dose of the lipocalin mutein may be about 10 mg or less, about 9 mg or less, about 8 mg or less, about 7 mg or less, about 6 mg or less, or about 5 mg, or about 2 mg, or about 1 mg, or about 100 μg, or about 50 μg or less per administration, or about 200 μg or less, about 180 μg or less, about 160 μg or less, about 140 μg or less, about 120 μg or less, about 100 μg or less, about 90 μg or less, about 80 μg or less, about 70 μg or less, about 60 μg or less, about 50 μg or less, about 40 μg or less, about 30 μg or less, about 20 μg or less, or about 10 μg or less per kg body weight per administration. The delivered dose of the lipocalin mutein may be about 0.05 mg or more, about 0.1 mg or more, or about 0.2 mg or more per administration, or about 0.05 μg or more, about 0.1 μg or more, about 0.15 μg or more per kg body weight per administration.

Local exposure may be desired if the lipocalin mutein is for use in the treatment of a disease or disorder of the respiratory tract. Local exposure will have the benefit that the lipocalin mutein remains at the place where it takes effect. Further, clearance rates of lipocalin muteins that remain in the respiratory tract may be lower than systemically absorbed lipocalin muteins.

In some embodiments, systemic exposure means that about 0.3% or more, about 0.4% or more, about 0.5% or more, about 0.6% or more, about 0.7% or more, about 0.8% or more, about 0.9% or more, about 1% or more, about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, or about 15% or more of the delivered dose of the lipocalin mutein enters circulatory system.

In order to achieve systemic exposure, the delivered dose of the lipocalin mutein may be about 0.05 mg to about 1000 mg per administration, preferably 5 mg to about 1000 mg per administration, preferably about 6 mg to about 500 mg per administration, preferably about 7 mg to about 300 mg per administration, preferably about 7 mg to about 280 mg per administration. The delivered dose of the lipocalin mutein may be about 0.1 μg to about 15 mg per kg body weight per administration, preferably about 0.05 mg to about 8 mg per kg body weight per administration, preferably about 0.1 mg to about 4 mg per kg body weight per administration. In general, the delivered dose of the lipocalin mutein may be about 4 mg or more, about 5 mg or more, about 6 mg or more, about 7 mg or more, about 8 mg or more, about 9 mg or more, about 10 mg or more, about 15 mg or more, about 20 mg or more, about 25 mg or more, about 30 mg or more, about 50 mg or more, or about 100 mg or more per administration or about 50 μg or more, 60 μg or more, 70 μg or more, 80 μg or more, about 90 μg or more, about 100 μg or more, about 120 μg or more, about 140 μg or more, about 160 μg or more, about 180 μg or more, about 200 μg or more, about 250 μg or more, about 300 μg or more, about 400 μg or more, about 500 μg or more per kg body weight per administration. The delivered dose of the lipocalin mutein may be about 400 mg or less, about 300 mg or less, about 200 mg or less, about 150 mg or less, about 120 mg or less, or about 100 mg or less per administration or about 6 mg or less, about 5 mg or less, about 4 mg or less, about 3 mg or less, about 2.5 mg or less, or about 2 mg or less per kg body weight per administration.

Systemic exposure of the lipocalin mutein following the inhaled administration may be characterized by rapid absorption. Maximum concentration of the lipocalin mutein in blood plasma may be reached about 0.1 hours to about 10 hours after administration, preferably after about 0.5 hours to about 5 h, preferably after about 1 to about 2 h. Maximum concentration of lipocalin mutein in blood plasma (Cₘₐₓ) may be about 1 ng per mL or more, about 3 ng per mL or more, about 8 ng per mL or more, about 10 ng per mL or more, about 50 ng per mL or more, about 100 ng per mL or more, about 600 ng per mL or more, about 1,000 ng per mL or more, about 1,500 ng per mL or more, or about 2,000 ng per mL, such as from about 1 ng per mL to about 2,000 ng per mL, from about 1 ng per mL to about 600 ng per mL, or from about 1 ng per mL to about 100 ng per mL. The area under the curve of the serum concentration over the time (AUC_{inf}) of the lipocalin mutein may be about 10 h*ng/mL or more, about 20 h*ng/mL or more, about 70 h*ng/mL or more, about 100 h*ng/mL or more, about 500 h*ng/mL or more, about 1,000 h*ng/mL or more, about 5,000 h*ng/mL or more, about 10,000 h*ng/mL or more, or about 16,000 h*ng/mL or more, such as from about 10 h*ng/mL to about 16,000 h*ng/mL, from about 10 h*ng/mL to about 5,000 h*ng/mL, or from 20 h*ng/mL to about 5,000 h*ng/mL. The serum half-life (t_{1/2}) of the lipocalin mutein may be from about 2 hours to about 10 hours, such as about 5 hours, about 6 hours, about 7 hours, about 8 hours, about 9 hours, or about 10 hours.

Systemic exposure may be desired to achieve additive and/or synergistic effects with drugs that otherwise remain in the respiratory tract, i.e., enter the circulatory system at very low level (or below limit of quantification). Systemic exposure may also be desired if the lipocalin mutein is for use in the treatment of a disease or disorder that is systemic or that affects a tissue or organ other than the respiratory system. Systemic exposure may e.g. be desired for the administration of a CGRP-specific lipocalin mutein. A “systemic disease” as used herein is one that affects a number of organs and tissues or affects the body as a whole.

Local exposure will have the benefit that the lipocalin mutein remains at the place where it takes effect. Further, clearance rates of lipocalin muteins that remain in the respiratory tract may be lower than systemically absorbed lipocalin muteins.

C. Formulations

Lipocalin muteins for use in the present invention will usually be administered in the form of a pharmaceutical composition, which may comprise at least one component in addition to the specific binding member. Thus, pharmaceutical compositions for use in accordance with the present invention may comprise, in addition to active ingredient, a pharmaceutically acceptable excipient, carrier, buffer, stabiliser or other materials well known to those skilled in the art. Such materials should be non-toxic and should not interfere with the efficacy of the active ingredient. For example, the lipocalin mutein for use in accordance with the present invention may be formulated in an aqueous solution of phosphate buffered saline (PBS).

In some embodiments, a pharmaceutical composition for use in accordance with the present invention may comprise an excipient. In some embodiments, such an excipient may facilitate an inhaled drug, e.g., a lipocalin mutein of the disclosure, to reach the deep lung and/or the alveolar region of the lung. In some embodiments, such an excipient may enhance the systemic uptake of an inhaled drug, e.g., a lipocalin mutein of the disclosure. In some embodiments, such an excipient may facilitate a faster onset of an inhaled drug, e.g., a lipocalin mutein of the disclosure. In some embodiments, such an excipient may contribute to enhanced therapeutic effects of an inhaled drug, e.g., a lipocalin mutein of the disclosure. In some embodiments, such an excipient may form microspheres in solution. In some embodiments, a pharmaceutical composition for use in accordance with the present invention may comprise fumaryl diketopiperazine (FDKP).

The pharmaceutical composition comprising the lipocalin mutein may be administered alone or in combination with other treatments, either simultaneously or sequentially.

Formulations suitable for use with a nebulizer typically comprise a lipocalin mutein dispersed in water or a liquid (usually aqueous) medium. The formulation may also include a buffer, a sugar (e.g., for protein stabilization and regulation of osmotic pressure), a (physiologic amount of a) salt, and/or other pharmaceutically acceptable excipients. Examples of buffers which may be used are phosphate, acetate, citrate and glycine. A suitable buffer is phosphate buffered saline (e.g. 1.06 mM KH₂PO₄, 2.96 mM Na₂HPO₄, 154 mM NaCl, pH 7.4).

Lipocalin mutein formulations for use with a metered-dose inhaler device typically comprise a finely divided powder. This powder may be produced by lyophilizing a lipocalin mutein containing formulation and milling to the desired particle size. The formulation may also contain a stabilizer such as human serum albumin (HSA). One or more sugars or sugar alcohols may be added to the preparation. Examples include lactose maltose, mannitol, sorbitol, sorbitose, trehalose, xylitol, and xylose. The particles may then be suspended in a propellant optionally with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

D. Treatment of Diseases

1. Diseases or Disorders of the Respiratory Tract

Diseases or disorders of the respiratory tract refer to any disease or disorder that involves the respiratory system. Such diseases or disorders may be treated using a lipocalin mutein via inhaled administration. In some embodiments, the administration may provide for local exposure to the lipocalin mutein in the respiratory tract. Local exposure may be beneficial allowing the lipocalin mutein to remain the respiratory tract, i.e., at the place where it takes effect. In some other embodiments, the administration may provide for systemic exposure to the lipocalin mutein. Such systemic exposure may provide additive and/or synergistic effects as compared to when the lipocalin mutein substantially remains in the respiratory tract, i.e., when systemic exposure is very low (i.e., below limit of quantification). In some embodiments, inhaled administration of lipocalin mutein provides an improved effect as compared to systemically administering (about) the same or a comparable bioavailable amount of the lipocalin mutein, such as potential longer duration of action. Accordingly, in some embodiments, systemic exposure to the inhaled lipocalin mutein may be desired.

Diseases or disorders of the respiratory tract include allergic inflammation, allergic asthma, rhinitis, conjunctivitis, lung fibrosis, cystic fibrosis, chronic obstructive pulmonary disease, pulmonary alveolar proteinosis, adult respiratory distress syndrome, or bacterial infections, such as, Pseudomonas aeruginosa infections. A disease or disorder of the respiratory tract may be a lung disorder, such as (allergic) asthma, chronic obstructive pulmonary disease (COPD) or cystic fibrosis (CF).

Interleukin (IL)-4 and IL-13 have long been associated with various diseases or disorders of the respiratory tract. Such diseases or disorders may be treated using an IL-4Rα antagonist, such as a lipocalin mutein specific for IL-4Rα.

For example, asthma is a complex, persistent, inflammatory disease characterized by airway hyper-responsiveness in association with airway inflammation. Studies suggest that regular use of high-dose inhaled corticosteroids and long-acting bronchodilators or omalizumab (a humanized monoclonal antibody that binds to immunoglobulin E and is often used as a step-up therapy for patients uncontrolled on standard of care therapy) may not be sufficient to provide asthma control in all patients, highlighting an important unmet need. Interleukin-4 (IL-4), interleukin-13 (IL-13), and the signal transducer and activator of transcription factor-6 are key components in the development of airway inflammation, mucus production, and airway hyper-responsiveness in asthma. In some preferred embodiments, the allergic asthma is an airway inflammation in which the IL-4/IL-13 pathway contributes to disease pathogenesis.

Additional lung disorders involving IL-4/IL-13 signaling pathways include pulmonary disorders. Such pulmonary disorders include but are not limited to, lung fibrosis, including chronic fibrotic lung disease, conditions characterized by IL-4-induced fibroblast proliferation or collagen accumulation in the lungs, pulmonary conditions in which a Th2 immune response plays a role, conditions characterized by decreased barrier function in the lung (e.g., resulting from IL-4-induced damage to the epithelium), or conditions in which IL-4 plays a role in an inflammatory response.

Cystic fibrosis (CF) is characterized by the overproduction of mucus and development of chronic infections. Inhibiting IL-4Rα and the Th2 response will reduce mucus production and help control infections such as allergic bronchopulmonary aspergillosis (ABPA). Allergic bronchopulmonary mycosis occurs primarily in patients with cystic fibrosis or asthma, where a Th2 immune response is dominant. Inhibiting IL-4Rα and the Th2 response will help clear and control these infections.

Chronic obstructive pulmonary disease (COPD) is associated with mucus hypersecretion and fibrosis. Inhibiting IL-4Rα and the Th2 response will reduce the production of mucus and the development of fibrous thereby improving respiratory function and delaying disease progression. Bleomycin-induced pneumopathy and fibrosis, and radiation-induced pulmonary fibrosis are disorders characterized by fibrosis of the lung which is manifested by the influx of Th2, CD4.sup.+ cells and macrophages, which produce IL-4 and IL-13 which in turn mediates the development of fibrosis. Inhibiting IL-4Rα and the Th2 response will reduce or prevent the development of these disorders.

Moreover, IL-4 and IL-13 induce the differentiation of lung epithelial cells into mucus-producing goblet cells. IL-4 and IL-13 may therefore contribute to an enhanced production of mucus in subpopulations or some situations. Mucus production and secretion contributes to disease pathogenesis in COPD and CF. Thus, the disorder, associated with a mucus production or a mucus secretion (for example, overproduction or hypersecretion), can be preferably treated, ameliorated or prevented by the methods of the present disclosure by applying a lipocalin mutein specific for IL-4Rα as described herein. In some preferred embodiments, the disorder, associated with a mucus production or a mucus secretion is preferably a chronic obstructive pulmonary disease (COPD) or a cystic fibrosis (CF).

Pulmonary alveolar proteinosis is characterized by the disruption of surfactant clearance. IL-4 increases surfactant product. In some further embodiments, use of an IL-4Rα antagonist such as a lipocalin mutein specific for IL-4Rα of the disclosure to decrease surfactant production and decrease the need for whole lung lavage, is also contemplated herein.

Adult respiratory distress syndrome (ARDS) may be attributable to a number of factors, one of which is exposure to toxic chemicals. Therefore, as a preferred but non-limiting example, one patient population susceptible to ARDS is critically ill patients who go on ventilators, as ARDS is a frequent complication in such patients. In some further embodiments, an IL-4Rα antagonist such as an IL-4Rα specific lipocalin mutein of the disclosure may thus be used to alleviate, prevent or treat ARDS by reducing inflammation and adhesion molecules.

Sarcoidosis is characterized by granulomatous lesions. In some further embodiments, use of an IL-4Rα antagonist such as an IL-4Rα specific lipocalin mutein of the disclosure to treat sarcoidosis, particularly pulmonary sarcoidosis, is also contemplated herein.

Conditions in which IL-4-induced barrier disruption in the lung plays a role may be treated with IL-4Rα antagonist(s). Damage to the epithelial barrier in the lungs may be induced by IL-4 and/or IL-13 directly or indirectly. The epithelium in the lung functions as a selective barrier that prevents contents of the lung lumen from entering the submucosa. A damaged or “leaky” barrier allows antigens to cross the barrier, which in turn elicits an immune response that may cause further damage to lung tissue. Such an immune response may include recruitment of eosinophils or mast cells, for example. An IL-4Rα antagonist may be locally administered to inhibit such undesirable stimulation of an immune response.

In this regard, an IL-4Rα antagonist such as an IL-4Rα specific lipocalin mutein of the disclosure may be employed to promote healing of lung epithelium, in asthmatics for example, thus restoring barrier function, or alternatively, administered for prophylactic purposes, to prevent IL-4 and/or IL-13-induced damage to lung epithelium, by local administration in the respiratory system.

The disease or disorder of the respiratory tract may be also a bacterial infection, such as an infection caused by the bacterium Pseudomonas aeruginosa (P. aeruginosa). P. aeruginosa is an opportunistic pathogen that causes acute infections, primarily in association with tissue injuries. Remarkably, the same pathogen is also associated with progressive and ultimately chronic recurrent respiratory infections in COPD, CF, bronchiectasis, and chronic destroyed lung disease (Yum et al., Tuberc Respir Dis (Seoul), 2014). The pathogenesis of P. aeruginosa infections largely depends on its ability to form biofilms, structured bacterial communities that can coat mucosal surfaces or invasive devices. Biofilm infections are difficult to treat with conventional antibiotic therapies. Pyoverdins and pyochelin are targets which are crucial for P. aeruginosa's pathogenicity. Accordingly, bacterial infections such as the ones caused by P. aeruginosa further represent diseases which may be treated via local exposure to lipocalin muteins of the disclosure following inhaled administration. Lipocalin muteins specific for pyoverdine type I, II, III or pyochelin may thus be used for the treatment of such infections.

Cancer treatment is another filed of application for achieving local exposure to lipocalin muteins, in particular treatment of cancers of the respiratory tract, such as lung cancer. Lipocalin muteins specific for a series of cancer targets are disclosed herein, and the use of all these lipocalin muteins in cancer treatment by inhaled administration is contemplated by the present disclosure. Such lipocalin muteins include lipocalin muteins specific for ED-B fibronectin, CTLA-4, c-Met, glypican-3, LAG-3, CD137, Ang-2, or VEGF.

2. Systemic Diseases and Diseases of Other Organs or Tissues

Systemic diseases or disorders that affect an organ or tissue other than the respiratory system may be treated using a lipocalin mutein that is systemically absorbed after inhaled administration. Such diseases or disorders include pain disorders, such as migraine, anemia, cardiovascular diseases, neurodegenerative diseases, such as Alzheimer's disease, inflammatory diseases, allergic diseases, cancer, and bacterial infections, such as P. aeruginosa infections.

Further to diseases or disorders of the respiratory tract, the IL-4/IL-13 pathway is also involved in a series of systemic diseases or diseases that affect other organs or tissues other than the respiratory tract. Examples for such diseases or disorders are allergic diseases, such as rhinitis, conjunctivitis, dermatitis or food allergies. Accordingly, there are also therapeutic applications for inhaled administration of IL-4 Rα specific lipocalin muteins that results in systemic exposure. The present disclosure therefore also contemplates treatment or prevention of diseases and disorders via systemic exposure to IL-4Rα specific lipocalin muteins. Such diseases or disorders include allergic diseases, such as rhinitis, conjunctivitis, dermatitis, or food allergies.

A pain disorder may be migraine, which is a primary headache disorder typically characterized by recurrent headaches that are moderate to severe. Typically, the headaches affect one half of the head, are pulsating in nature, and last from two to 72 hours. Associated symptoms may include nausea, vomiting, and sensitivity to light, sound, or smell. CGRP has been reported to play a role in migraines as CGRP is released upon stimulation of sensory nerves and has potent vasodilatory activity (Arulmozhi et al., Vascul Pharmacol, 2005). Further, the release of CGRP increases vascular permeability and subsequent plasma protein leakage (plasma protein extravasation) in tissues innervated by trigeminal nerve fibers upon stimulation of these fibers (Arulmozhi et al., Vascul Pharmacol, 2005). In addition, studies have reported that infusion of CGRP in patients who suffer from migraines has resulted in migraine-like symptoms (Lassen et al., Cephalalgia, 2002). CGRP specific lipocalin muteins of the disclosure may be used for the treatment of diseases or disorders associated with deregulated levels of free CGRP. Such lipocalin muteins may be used to decrease circulating levels of free CGRP. Preferably, a CRGP binding lipocalin mutein of the disclosure may be useful for the treatment, prevention, and/or amelioration of a parin disorder, in particular migraine. Inhaled administration of CGRP specific lipocalin muteins has the advantage that the method avoids injections and enables self-medication. A further advantage is the fast onset of the therapeutic efficacy and systemic absorption when administering a CGRP specific lipocalin mutein by inhalation.

Surprisingly, inhaled administration of a lipocalin mutein, such as a CGRP-specific lipocalin mutein described herein, may have an onset that is superior to (i.e. faster than) subcutaneous administration. Surprisingly, inhaled administration of a lipocalin mutein, such as a CGRP specific lipocalin mutein described herein, may have an onset that is comparable (i.e. about as fast as) or even faster than direct systemic administration, such as intravenous administration. Increased systemic exposure, more rapid onset, and/or enhanced therapeutic effect of a lipocalin mutein may be achieved through the formulation with certain excipients, such as fumaryl diketopiperazine (FDKP). In particular with regard to CGRP-specific lipocalin muteins of the disclosure (for example a lipocalin mutein comprising the sequence set forth in SEQ ID NO: 47), the onset may be about as fast as or even faster than a reference anti-CGRP antibody (SEQ ID NOs: 204 and 205). When formulated with FDKP, e.g., at 0.4 mg/kg, a CGRP specific lipocalin mutein of the disclosure may display more rapid onset, such as an onset of about 1 minute, about 2 minutes, about 3 minutes, about 4 minutes, about 5 minutes, about 10 minutes, about 15 minutes, about 20 minutes, about 25 minutes, or about 30 minutes. The onset of a CGRP specific lipocalin mutein may be determined in a sensory nerve-mediated vasodilatation assay. Such an assay may be conducted as essentially described in Example 4.

The therapeutic effect of a CGRP-specific lipocalin mutein (for example a lipocalin mutein comprising the sequence set forth in SEQ ID NO: 47) described herein, such as the inhibition of vasodilation, is comparable or superior to a reference anti-CGRP antibody (SEQ ID NOs: 204 and 205). When formulated with FDKP, e.g., at 0.4 mg/kg, the onset of a CGRP specific lipocalin mutein of the disclosure may be further enhanced.

Anemia is a disease associated with serum iron depletion leading to a decrease of hematological parameters such as red blood cell (RBC) counts, hematocrit (Ht), hemoglobin (Hb), serum iron level and transferrin (Tf) saturation. This results in a decreased oxygen level in the blood and is associated with a declined quality of life (QOL) described by weakness, poor concentration, shortness of breath and dyspnea. Severe anemia can lead to a fast heart rate, cardiac enlargement and heart failure. Anemia is often associated with chronic kidney disease/established chronic kidney disease (CKD), anemia of cancer (AC), chemotherapy induced anemia (CIA) and anemia of chronic disease (ACD).

Iron deficiency anemia is a disorder of iron homeostasis that is easily cured by iron administration in contrast to anemia associated with inflammatory disease. Hepcidin is a parameter that allows distinguishing between these two disorders since the hepcidin level is only upregulated in combination with inflammation.

Hepcidin is the central negative regulator of iron homeostasis. Hepcidin production increases with iron loading and inflammation and decreases under low iron conditions and hypoxia. Hepcidin acts via binding to the only known mammalian cellular iron exporter, ferroportin, and induces its internalization and degradation. Since ferroportin is expressed in the duodenal enterocytes, spleen, and liver, hepcidin increase, and the subsequent decrease of ferroportin, results in the inhibition of duodenal iron absorption, release of recycled iron from macrophages, and mobilization of iron stores in the liver. Hepcidin is thought to play a critical role in the development of anemia associated with inflammatory disease. Acute or chronic inflammatory conditions result in the upregulation of hepcidin expression, leading to iron deficiency, which can cause anemia associated with ACD, AC, CIA, and anemia of CKD.

A hepcidin binding lipocalin mutein of the disclosure may be used to treat a subject having an elevated level of hepcidin, a hepcidin-related disorder, a disorder of iron homeostasis, anemia or inflammatory condition associated with an elevated level of hepcidin. Anemia may be any of anemia of inflammation, chronic inflammatory anemia, an iron-deficiency anemia, an iron loading anemia, anemia associated with CKD, AC, CIA, or an anemia associated with erythropoiesis-stimulating agent (ESA)-resistance.

Coronary artery disease (CAD), also known as ischemic heart disease (IHD), involves the reduction of blood flow to the heart muscle due to build up of plaque in the arteries of the heart. It is the most common of the cardiovascular diseases. Types include stable angina, unstable angina, myocardial infarction, and sudden cardiac death. A PCSK9 binding lipocalin mutein of the disclosure may be used to treat or prevent such a coronary heart disease.

Neurodegenerative diseases are a further group of diseases that may be treated via systemic exposure to lipocalin muteins following inhaled administration. Alzheimer's disease (AD) is the most common form of dementia in the elderly population. Associated with AD is the defective processing of the amyloid precursor protein giving rise to the potentially neurotoxic, 40-42 residues encompassing amyloid beta peptide (Aβ). Subsequent aggregation of Aβ to oligomers and long fibrils plays a pivotal role in the course of the disease, culminating in the formation of senile plaques (Haass and Selkoe, Nat Rev Mol Cell Biol, 2007). An amyloid beta specific lipocalin mutein of the disclosure may thus be used to treat or prevent a neurodegenerative disease such as AD.

Bacterial infections such as the ones caused by P. aeruginosa further represent diseases which may be treated via systemic exposure to lipocalin muteins of the disclosure following inhaled administration. Pyoverdins and pyochelin are targets which are crucial for P. aeruginosa's pathogenicity. Lipocalin muteins specific for pyoverdine type I, II, III or pyochelin may thus be used for the treatment of such infections.

Inflammatory diseases and autoimmune diseases are a further group of diseases, which may be treated systemic exposure to lipocalin muteins of the disclosure following inhaled administration. Both IL-17A and IL-23 are cytokines involved in inflammation and autoimmune diseases. IL-17A specific or IL-23 specific lipocalin muteins of the disclosure may thus be used for the treatment or prevention of inflammatory diseases or autoimmune diseases, such as multiple sclerosis, rheumatoid arthritis, Crohn's disease, and psoriasis.

Cancer treatment is another filed of application for achieving systemic exposure to lipocalin muteins. Lipocalin muteins specific for a series of cancer targets are disclosed herein, and the use of all these lipocalin muteins in cancer treatment by inhaled administration is contemplated by the present disclosure. Such lipocalin muteins include lipocalin muteins specific for ED-B fibronectin, CTLA-4, c-Met, glypican-3, LAG-3, CD137, Ang-2, or VEGF.

Additional objects, advantages, and features of this disclosure will become apparent to those skilled in the art upon examination of the following Examples and the attached Figures thereof, which are not intended to be limiting. Thus, it should be understood that although the present disclosure is specifically disclosed by exemplary embodiments and optional features, modification and variation of the disclosures embodied therein herein disclosed may be resorted to by those skilled in the art, and that such modifications and variations are considered to be within the scope of this disclosure.

### V. EXAMPLES

### Example 1

### Pharmacokinetics of Intratracheally Administered Lipocalin Muteins In Healthy Mice

Analyses of the pharmacokinetics (PK) of exemplary lipocalin muteins (SEQ ID NOs: 3 and 4) were performed in healthy mice.

Female mice approximately 8 weeks of age were intratracheally administered respective test lipocalin mutein at a dose of 100 μg/kg or 100 μg/mouse using a microsprayer (#1A-1C-M, Penn Century). Blood plasma, bronchoalveolar lavage fluid (BALF), and lung homogenate samples were obtained after sacrificing mice at 1 h, 4 h, 6 h, 12 hours and 24 hours (n=5 animals per timepoint per test molecule), and the corresponding drug levels were determined.

Blood was drawn at the determined time points from all animals in the experimental group by cardiac puncture under light Isoflurane anesthesia into tubes with lithium heparin as the anti-coagulant. Samples were then centrifuged for 10 minutes at 5000×rpm in an Eppendorf tube at 4° C. and plasma was collected (100 μL/tube) and stored at −80 ° C. until further use. BALF sample was obtained by washing the lungs with 0.5 ml saline for three times (total: 1.5 ml), followed by centrifugation at 400×g for 10 minutes at 4° C. The supernatant was collected and stored at −80° C. until being assayed. Immediately after lavage, the lung was perfused through the right heart ventricle with saline to flush the vascular content and subsequently frozen and stored at −20° C. The lung homogenate was obtained by homogenizing weighted lung in 1 mL PBS with protease inhibitor cocktail using an Ultra Turrax Homogenizer (IKA). The lung homogenate was aliquoted and stored at −80° C. for further analyses. Before the analyses, the total protein concentration in the lung homogenate was quantified using a BCA Protein Assay Kit. Homogenate samples were adjusted to a total protein concentration of 5 mg/mL with PBS (normalized lung homogenate).

Drug levels in BALF, lung homogenate, and plasma were analyzed using the following protocol: anti-NGAL or anti-Tlc antibody (Pieris) was dissolved in PBS (1 μg/mL) and coated overnight on microtiter plates at 4° C. The plates were washed after each incubation step with 80 μL PBS-0.05% T (PBS supplemented with 0.05% (v/v) Tween 20) for five times. The plates were blocked with 2% BSA (w/v) in PBS-0.1% T (PBS-0.1% T-2% BSA) for 1 hours at room temperature and subsequently washed. Samples were diluted in PBS-0.1% T-2% BSA —plasma samples to 50% plasma concentration, lung samples to 20% lung homogenate, and BALF samples to 20% BALF in FACS buffer—added to the wells and incubated for 1 hours at room temperature. Another wash step followed. Bound agents under study were detected after 1 hours incubation with anti-NGAL-biotin or anti-Tlc-biotin at 1 μg/mL and SULFO-tag streptavidin (Meso Scale Discovery) at 1 μg/mL, each diluted in PBS-0.1% T-2% BSA. After an additional wash step, MSD Read Buffer with surfactant was added to each well and the electrochemiluminescence (ECL) signal of every well was read using a Meso Scale Discovery reader. For data analyses and quantification, a calibration curve with standard protein dilutions was also prepared.

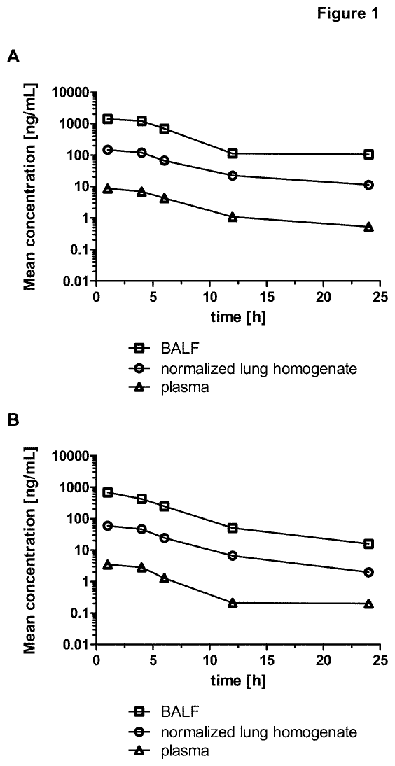

The BALF, normalized lung homogenate, and plasma concentrations of the test molecules over time following intratracheal administration in an exemplary experiment were plotted in FIGS. 1A and 1B. A non-compartmental analysis was applied to the data using Phoenix WinNonlin version 8.1, and the calculated half-lives are summarized in Table 1. The data show that similar PK profiles were observed for SEQ ID NO: 3 (lipocalin mutein of hNGAL) and SEQ ID NO: 4 (lipocalin mutein of hTlc) in each of the three compartments (BALF, lung, and plasma) upon single intratracheal lung dosing.

TABLE 1
PK parameters following lipocalin mutein intratracheal administration

| | | | Normalized Lung | | | |
|---|---|---|---|---|---|---|
| | BALF | | Homogenate | | Plasma | |
| | SEQ | SEQ | SEQ | SEQ | SEQ | SEQ |
| Parameter | ID NO: 3 | ID NO: 4 | ID NO: 3 | ID NO: 4 | ID NO: 3 | ID NO: 4 |
| t_{1/2} [h] | 5.9 | 4.3 | 6.2 | 4.6 | 5.52 | 5.59 |
| Tₘₐₓ [h] | 1 | 1 | 1 | 1 | 1 | 1 |
| Cₘₐₓ [ng/mL] | 1411.35 | 680.89 | 147.28 | 58.9 | 8.7 | 3.5 |
| Tₗₐₛₜ [h] | 24 | 24 | 24 | 24 | 24 | 24 |
| Cₗₐₛₜ [ng/mL] | 105.78 | 15.81 | 11.26 | 1.98 | 0.53 | 0.2 |
| AUCₗₐₛₜ [h * ng/mL] | 10225.4 | 3953.24 | 1129.48 | 403.71 | 65 | 22.37 |
| AUC_{inf} [h * ng/mL] | 11127.29 | 4051.3 | 1230.19 | 416.77 | 69.22 | 23.98 |
| Vz/F [mL/kg] | 76.62 | 153.09 | 726.97 | 1580.36 | 11513 | 33643.65 |
| CL/F [mL/h/kg] | 8.9869 | 24.6835 | 81.29 | 239.94 | 1444.74 | 4169.38 |

### Example 2

### Pharmacokinetics of Intratracheally Administered Lipocalin Muteins in Healthy Mice

Similar PK analyses were also performed with male BALB/c mice approximately 8 weeks of age, where animals were intratracheally administered lipocalin mutein SEQ ID NO: 4 (lipocalin mutein of hTlc) at a dose of 84 μg/mouse and SEQ ID NO: 3 (lipocalin mutein of hNGAL) at a dose of 100 μg/mouse using a microsprayer. Animals were sacrificed mice at 1 h, 2 h, 4 h, 6 h, 24 h, and 48 hours (n=5 animals per timepoint per test molecule) by overdosing with intraperitoneal injection of sodium pentobarbitone (200 mg/kg of body weight or 200 μL of 80 mg/mL stock solution), and blood plasma, BALF, and lung tissues were collected for PK analyses. For the timepoints between 1 to 6 hours, mice intratracheal injections were spaced 20 minutes apart; for the 24 hours and 48 hours timepoints, the mice were injected 5 minutes apart.

Following the surgical resection with pneumothorax, approximately 0.5 mL cardiac blood was drawn into a 1.5 mL tube containing 20 μL of 0.5 M EDTA, by inserting a 30 G needle with a syringe into the heart below the atrium. For blood plasma isolation, the blood samples were then centrifuged at 3000×g for 10 minutes at 4° C. The plasma was then collected and frozen until analysis.

For BALF collection, a BALF harvest tip was inserted into the mouse trachea to repeatedly inject PBS, and then BALF was drawn into a sterile tube to 250 to 300 μL and labeled as “BALF Wash 1”. The lung was washed for another 7 times, each time with 300 μL PBS and BALF collected into separate tubes as BALF Wash 2-8. The BALF washes were stored at −80° C. until further analyses.

To harvest lung tissues, the individual lobes (Lobe 1: left lobe; Lobe 2: inferior lobe; Lobe 3: superior lobe; Lobe 4: middle lobe) were cut, weighted, and frozen on dry ice. Lung lobes were homogenized using a TissueLyser LT apparatus (Qiagen) in RIPA buffer (50 mM Tris-HCl, pH 7.4, 1% Triton X-100, 0.2% sodium deoxycholate, 0.2% sodium dodecylsulfate with Complete Protease Inhibitor Cocktail) and centrifuged at 10000×g for 10 minutes at 4° C. The supernatant was collected and quantified for protein concentration using a BCA Protein assay Kit. Homogenate samples were adjusted to a protein concentration of 5 mg/mL with RIPA buffer and stored for further use (normalized lung homogenate).

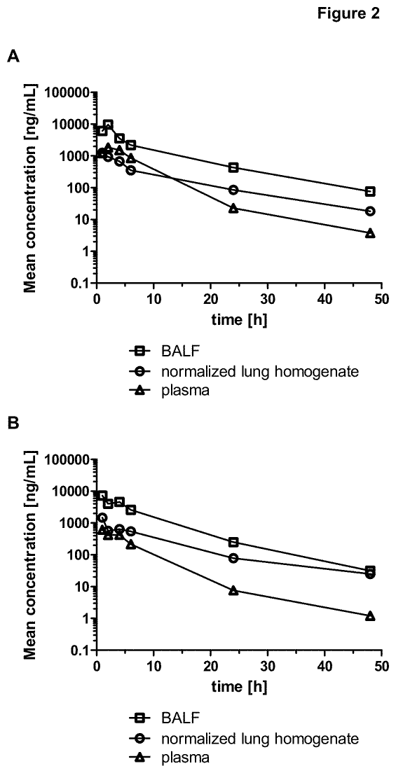

Drug levels in different compartments were determined using ELISA, as described above in Example 1. The mean concentrations of the test molecules in BALF, lung, or plasma over time were plotted. The results of an exemplary experiment are shown in FIGS. 2A and 2B. The corresponding PK parameters are summarized in Table 2.

The data show that similar time-dependent decreases in concentration were observed for SEQ ID NO: 4 (lipocalin mutein of hTlc) and SEQ ID NO: 3 (lipocalin mutein of hNGAL) in all three compartments—BALF, lung tissues, and plasma. The exposure levels are highest in the BALF for both lipocalins, displaying ˜4-fold or ˜12-fold higher levels as compared to the plasma exposures for SEQ ID NO: 3 (lipocalin mutein of hNGAL) or SEQ ID NO: 4 (lipocalin mutein of hTlc), respectively. The same trend was seen for Cₘₐₓ.

TABLE 2
PK parameters in lung and periphery following lipocalin mutein intratracheal administration

| | | | Normalized Lung | | | |
|---|---|---|---|---|---|---|
| | BALF | | Homogenate | | Plasma | |
| | SEQ ID | SEQ ID | SEQ ID | SEQ ID | SEQ ID | SEQ ID |
| Parameter | NO: 3 | NO: 4 | NO: 3 | NO: 4 | NO: 3 | NO: 4 |
| t_{1/2} [h] | 8.19 | 6.24 | 8.95 | 9.20 | 5.06 | 5.24 |
| Tₘₐₓ [h] | 2.00 | 1.00 | 1.00 | 1.00 | 2.00 | 1.00 |
| Cₘₐₓ [ng/mL] | 9740.12 | 7294.63 | 1261.08 | 1465.53 | 1859.23 | 616.3 |
| Tₗₐₛₜ [h] | 48.00 | 48.00 | 48.00 | 48.00 | 48.00 | 48.00 |
| Cₗₐₛₜ [ng/mL] | 76.45 | 31.54 | 18.36 | 25.05 | 3.78 | 1.21 |
| AUCₗₐₛₜ [h * ng/mL] | 59799.58 | 53974.34 | 9544.88 | 11012.19 | 16164.97 | 4431.52 |
| AUC_{inf} [h * ng/mL] | 60702.38 | 54258.4 | 9782.04 | 11344.75 | 16192.56 | 4440.66 |
| Vz/F [mL/kg] | 19.45 | 16.6 | 132.05 | 117.02 | 45.09 | 170.22 |
| CL/F [mL/h/kg] | 1.64738 | 1.84303 | 10.22282 | 8.81465 | 6.17568 | 22.51917 |

### Example 3

### Pharmacokinetics of Intravenously Administered Lipocalin Muteins in Healthy Mice

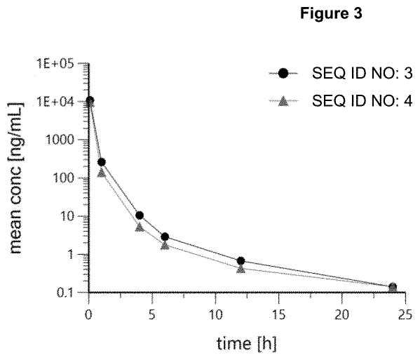

Additional male BALB/c mice were administered an intravenous dose of 2 mg/kg of the test lipocalin mutein (SEQ ID NO: 4 or SEQ ID NO: 3). Mice were sacrificed at 5 minutes, 1 h, 6 h, 12 h, and 24 hours and drug levels evaluated in blood plasma (n=2 animals per timepoint per test molecule). For an exemplary example, the drug concentrations over time were plotted as shown in FIG. 3 and the corresponding PK parameters are summarized in Table 3. Accordingly, the bioavailability of the lipocalin mutein may be determined by comparing the AUC_{inf} following intratracheal administration and ed inhalation with the AUC_{inf} for intravenous group.

TABLE 3
Serum PK parameters following
lipocalin mutein intravenous administration

| Parameter | SEQ ID NO: 3 | SEQ ID NO: 4 |
|---|---|---|
| t_{1/2} [h] | 2.57 | 3.52 |
| Tₘₐₓ [h] | 0.08 | 0.08 |
| Tₗₐₛₜ [h] | 24.00 | 24.00 |
| Cₘₐₓ [ng/mL] | 33903.17 | 10825.97 |
| AUCₗₐₛₜ [h * ng/mL] | 28078.85 | 6600.38 |
| AUC_{inf} [h * ng/mL] | 28079.82 | 6600.95 |
| Vz/F [mL/kg] | 263.88 | 1538.73 |
| Cl/F [mL/h/kg] | 71.22 | 302.98 |

### Example 4

### Assessment of the Effects of an Anti-CGRP Lipocalin Mutein SEQ ID NO: 47 Sensory Nerve-Mediated Skin Vasodilatation in the Rat Hindpaw in Anaesthetised Male Sprague-Dawley Rats

In order to investigate the in vivo effect of inhaled lipocalin mutein, a skin vasodilation model (Zeller et al., Br J Pharmacol, 2008) was used where rats were treated with a single dose of an exemplary lipocalin mutein (SEQ ID NO: 47) and the skin vasodilatation measured after saphenous nerve stimulation.

Male Sprague Dawley rats (approx. 300 g body weight) were anaesthetized with intraperitoneal (i.p.) urethane injection. The anaesthetized rat was placed on a homeostatic blanket system to maintain body temperature and ventilated via a tracheotomy with pO₂, pCO₂ & pH maintained via arterial blood gas analyses (50 μl blood samples). Wherever necessary, ventilator adjustment was performed. Atropine was administered subcutaneously (s.c.) to inhibit bronchial secretions.

Following the initial preparation, the saphenous nerve of one hindlimb was exposed via a small incision and a bipolar platinum electrode was positioned for subsequent antidromic electrical stimulation of the sensory nerve fibers that run together with the saphenous nerve (the nerve was cut and bretylium used to block the sympathetic nerves). A loose cover was arranged around the animal and the exposed hindlimbs to maintain a constant ambient temperature throughout the measurement period. Skin blood flow was measured via a laser Doppler probe placed on the hindpaw.

At defined time point prior to the nerve stimulation, the anti-CGRP lipocalin mutein (SEQ ID NO: 47) was intravenously administered at a dose of 1 mg/kg, subcutaneously administered at a dose of 5 mg/kg, intratracheally administered via a microsprayer device at a dose of 5 mg/kg, or intratracheally administered via a microsprayer device with a lung penetration enhancer fumaryl diketopiperazine at a dose of 5 mg/kg. Additionally, the test was performed with the anti-CGRP lipocalin mutein (SEQ ID NO: 47) intravenously-administered at a dose of 1 mg/kg, 2.5 mg/kg, 5 mg/kg, or 10 mg/kg, or intratracheally-administered via a microsprayer device at a dose of 2.5 mg/kg, 5 mg/kg, or 10 mg/kg. As a control, a reference anti-CGRP antibody (SEQ ID NOs: 204 and 205) was also tested via intravenous administration.

In addition to skin blood flow, mean arterial blood pressure (MAP) and heart rate (HR) was measured via a carotid arterial catheter. Carotid vascular resistance (MAP/carotid flow) was measured by placing a Transonic ultrasonic blood flow transducer (1 mm i.d., model #1PRB) on the contralateral carotid artery, which indicated any effects of the drug treatment on baseline haemodynamics (i.e. if the lipocalin mutein removed endogenous CGRP tone to cause vasoconstriction). Blood samples were taken at 5 minutes, 30 minutes, 60 minutes, and 120 minutes following the administration and drug pharmacokinetics was analyzed.

The rat was euthanized after the final observation point (2 hour) with an overdose of anaesthetic.

Measured parameters—HR, MAP, carotid vascular resistance, and laser Doppler hindpaw skin blood flow—were recorded via a Powerlab connected to a computer (Chart version 7, AD Instruments, Sydney, Australia). Data acquisition systems, Cobe blood pressure transducer and Transonic blood flow transducer, were calibrated on each experimental day.

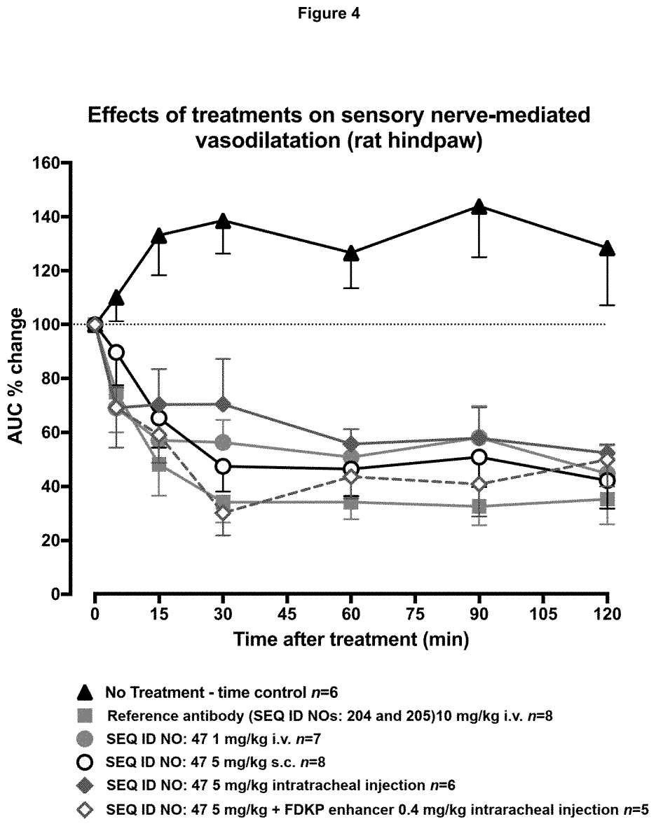

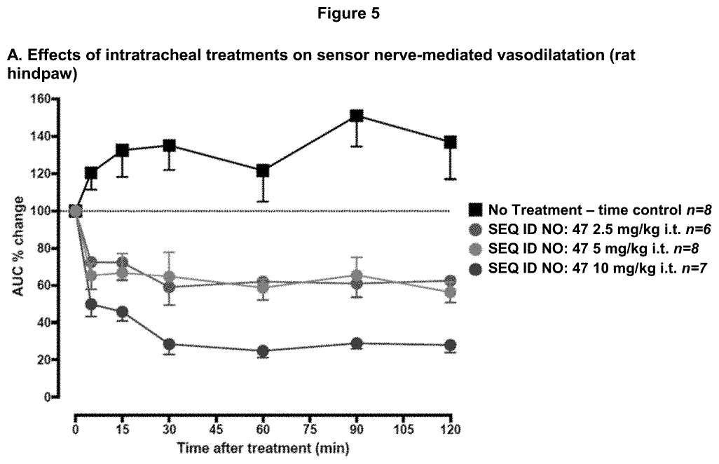

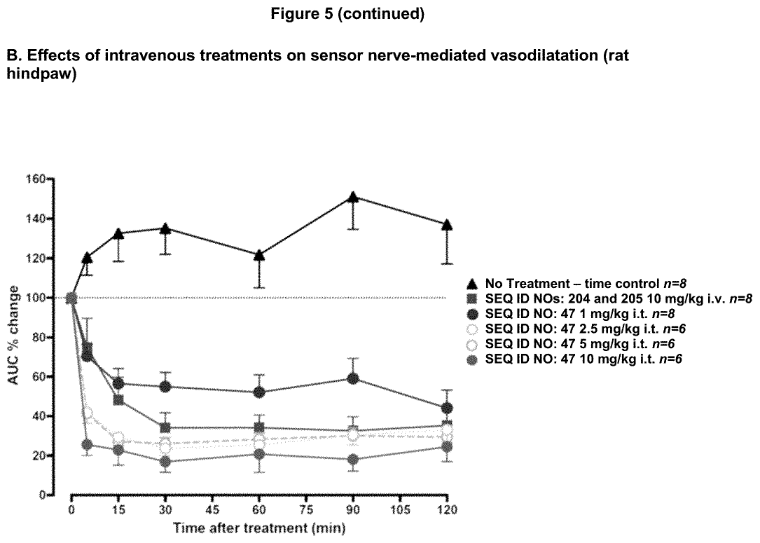

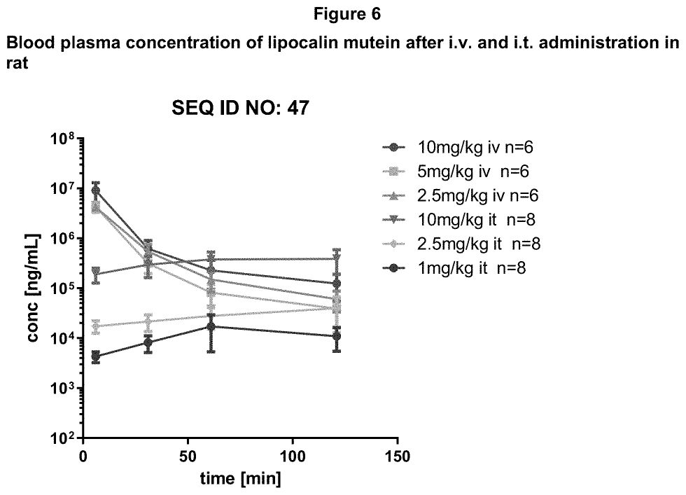

Results are shown in FIGS. 4 and 5. Blood plasma concentrations of anti-CGRP lipocalin mutein (SEQ ID NO: 47) in anti-CGRP lipocalin mutein-treated animals are shown in FIG. 6. In anti-CGRP lipocalin mutein (SEQ ID NO: 47)-treated animals, the dermal blood flow in the dorsomedial skin of the rat hind paw is significantly decreased from that seen in untreated animals, starting at about 5 minutes after dosing and lasting for at least 2 hours, indicating increased vasoconstriction through blocking CGRP. When formulated with 0.4 mg/kg fumaryl diketopiperazine (FDKP), SEQ ID NO: 47-induced decrease in vascular resistance is enhanced. The maximal change in carotid vascular resistance with SEQ ID NO: 47 after nerve stimulation is comparable to that observed of the reference anti-CGRP antibody (SEQ ID NOs: 204 and 205). The blood plasma levels of the anti-CGRP lipocalin mutein (SEQ ID NO: 47) correlate well with the PD effects seen in the skin vasodilatation experiment. Intratracheally administered lipocalin mutein displays faster onset than subcunateously administered lipocalin mutein and comparable or even faster onset as compared to intravenously administered lipocalin mutein or reference anti-CGRP antibody.

Embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present embodiments have been specifically disclosed by preferred embodiments and optional features, modification and variations thereof may be resorted to by those skilled in the art, and that such modifications and variations are considered to be within the scope of this invention. All patents, patent applications, textbooks, and peer-reviewed publications described herein are hereby incorporated by reference in their entirety. Furthermore, where a definition or use of a term in a reference, which is incorporated by reference herein is inconsistent or contrary to the definition of that term provided herein, the definition of that term provided herein applies and the definition of that term in the reference does not apply. Each of the narrower species and subgeneric groupings falling within the generic disclosure also forms part of the invention. This includes the generic description of the invention with a proviso or negative limitation removing any subject matter from the genus, regardless of whether or not the excised material is specifically recited herein. In addition, where features are described in terms of Markush groups, those skilled in the art will recognize that the disclosure is also thereby described in terms of any individual member or subgroup of members of the Markush group. Further embodiments will become apparent from the following claims.

Equivalents: Those skilled in the art will recognize, or be able to ascertain using no more than routine experimentation, many equivalents to the specific embodiments of the invention described herein. Such equivalents are intended to be encompassed by the following claims. All publications, patents, and patent applications mentioned in this specification are herein incorporated by reference into the specification to the same extent as if each individual publication, patent or patent application was specifically and individually indicated to be incorporated herein by reference.

### VI. NON-PATENT REFERENCES

1. AGU, R. U., UGWOKE, M. I., ARMAND, M., KINGET, R. & VERBEKE, N. 2001. The lung as a route for systemic delivery of therapeutic proteins and peptides. Respir Res, 2, 198-209.
2. SKERRA, A. 2000. Lipocalins as a scaffold. Biochim Biophys Acta, 1482, 337-50.
3. SKERRA, A. 2008. Alternative binding proteins: anticalins—harnessing the structural plasticity of the lipocalin ligand pocket to engineer novel binding activities. FEBS J, 275, 2677-83.
4. SCHLEHUBER, S. & SKERRA, A. 2002. Tuning ligand affinity, specificity, and folding stability of an engineered lipocalin variant—a so-called ‘anticalin’—using a molecular random approach. Biophys Chem, 96, 213-28.
5. SKERRA, A. 2001. ‘Anticalins’: a new class of engineered ligand-binding proteins with antibody-like properties. J Biotechnol, 74, 257-75.
6. ALTSCHUL, S. F., MADDEN, T. L., SCHAFFER, A. A., ZHANG, J., ZHANG, Z., MILLER, W. & LIPMAN, D. J. 1997. Gapped BLAST and PSI-BLAST: a new generation of protein database search programs. Nucleic Acids Res, 25, 3389-402.
7. ALTSCHUL, S. F., GISH, W., MILLER, W., MYERS, E. W. & LIPMAN, D. J. 1990. Basic local alignment search tool. J Mol Biol, 215, 403-10.
8. PEARSON, W. R. & LIPMAN, D. J. 1988. Improved tools for biological sequence comparison. Proc Natl Acad Sci USA, 85, 2444-8.
9. SMITH, T. F. & WATERMAN, M. S. 1981. Identification of common molecular subsequences. J Mol Biol, 147, 195-7.
10. WARD, E. S., GUSSOW, D., GRIFFITHS, A. D., JONES, P. T. & WINTER, G. 1989. Binding activities of a repertoire of single immunoglobulin variable domains secreted from Escherichia coli. Nature, 341, 544-6.
11. HOLLIGER, P., PROSPERO, T. & WINTER, G. 1993. “Diabodies”: small bivalent and bispecific antibody fragments. Proc Natl Acad Sci USA, 90, 6444-8.
12. BODIER-MONTAGUTELLI, E., MAYOR, A., VECELLIO, L., RESPAUD, R. & HEUZE-VOURC'H, N. 2018. Designing inhaled protein therapeutics for topical lung delivery: what are the next steps? Expert Opin Drug Deliv, 15, 729-736.
13. FLOWER, D. R., NORTH, A. C. & SANSOM, C. E. 2000. The lipocalin protein family: structural and sequence overview. Biochim Biophys Acta, 1482, 9-24.
14. FLOWER, D. R. 1996. The lipocalin protein family: structure and function. Biochem J, 318 (Pt 1), 1-14.
15. SCHMIDT, T. G., KOEPKE, J., FRANK, R. & SKERRA, A. 1996. Molecular interaction between the Strep-tag affinity peptide and its cognate target, streptavidin. J Mol Biol, 255, 753-66.
16. VENTURI, M., SEIFERT, C. & HUNTE, C. 2002. High level production of functional antibody Fab fragments in an oxidizing bacterial cytoplasm. J Mol Biol, 315, 1-8.
17. BRUCKDORFER, T., MARDER, O. & ALBERICIO, F. 2004. From production of peptides in milligram amounts for research to multi-tons quantities for drugs of the future. Curr Pharm Biotechnol, 5, 29-43.
18. NAURECKIENE, S., MA, L., SREEKUMAR, K., PURANDARE, U., LO, C. F., HUANG, Y., CHIANG, L. W., GRENIER, J. M., OZENBERGER, B. A., JACOBSEN, J. S., KENNEDY, J. D., DISTEFANO, P. S., WOOD, A. & BINGHAM, B. 2003. Functional characterization of Narc 1, a novel proteinase related to proteinase K. Arch Biochem Biophys, 420, 55-67.
19. PETERSON, A. S., FONG, L. G. & YOUNG, S. G. 2008. PCSK9 function and physiology. J Lipid Res, 49, 1595-9.
20. TAKASE, H., NITANAI, H., HOSHINO, K. & OTANI, T. 2000. Impact of siderophore production on Pseudomonas aeruginosa infections in immunosuppressed mice. Infect Immun, 68, 1834-9.
21. VISCA, P., COLOTTI, G., SERINO, L., VERZILI, D., ORSI, N. & CHIANCONE, E. 1992. Metal regulation of siderophore synthesis in Pseudomonas aeruginosa and functional effects of siderophore-metal complexes. Appl Environ Microbiol, 58, 2886-93.
22. G., B., K., T. & BUDZIKIEWICZ, H. 1989. Pyoverdin-type siderophores from Pseudomonas fluorescens. Liebigs Ann Chem, 375-384.
23. TAPPE, R., TARAZ, K., BUDZIKIEWICZ, H., MEYER, J. M. & LEFEVRE, J. F. 1993. Structure elucidation of a further pyoverdin produced by Pseudomonas aeruginosa ATCC 27853. J Prakt Chem, 83-87.
24. GIPP, S., HAHN, J., TARAZ, K. & BUDZIKIEWICZ, H. 1991. Zwei Pyoverdine aus Pseudomonas aeruginosa R. Z. Naturforsch, 534-541.
25. MEYER, J. M., STINTZI, A., DE VOS, D., CORNELIS, P., TAPPE, R., TARAZ, K. & BUDZIKIEWICZ, H. 1997. Use of siderophores to type pseudomonads: the three Pseudomonas aeruginosa pyoverdine systems. Microbiology, 143 (Pt 1), 35-43.
26. CORNELIS, P., HOHNADEL, D. & MEYER, J. M. 1989. Evidence for different pyoverdine-mediated iron uptake systems among Pseudomonas aeruginosa strains. Infect Immun, 57, 3491-7.
27. ZARDI, L., CARNEMOLLA, B., SIRI, A., PETERSEN, T. E., PAOLELLA, G., SEBASTIO, G. & BARALLE, F. E. 1987. Transformed human cells produce a new fibronectin isoform by preferential alternative splicing of a previously unobserved exon. EMBO J, 6, 2337-42.
28. SPRIGGS, M. K. 1997. Interleukin-17 and its receptor. J Clin Immunol, 17, 366-9.
29. MOSELEY, T. A., HAUDENSCHILD, D. R., ROSE, L. & REDDI, A. H. 2003. Interleukin-17 family and IL-17 receptors. Cytokine Growth Factor Rev, 14, 155-74.
30. KAWAGUCHI, M., ADACHI, M., ODA, N., KOKUBU, F. & HUANG, S. K. 2004. IL-17 cytokine family. J Allergy Clin Immunol, 114, 1265-73; quiz 1274.
31. KOLLS, J. K. & LINDEN, A. 2004. Interleukin-17 family members and inflammation. Immunity, 21, 467-76.
32. LUBBERTS, E. 2008. IL-17/Th17 targeting: on the road to prevent chronic destructive arthritis? Cytokine, 41, 84-91.
33. VAN KOOTEN, C., BOONSTRA, J. G., PAAPE, M. E., FOSSIEZ, F., BANCHEREAU, J., LEBECQUE, S., BRUIJN, J. A., DE FIJTER, J. W., VAN ES, L. A. & DAHA, M. R. 1998. Interleukin-17 activates human renal epithelial cells in vitro and is expressed during renal allograft rejection. J Am Soc Nephrol, 9, 1526-34.
34. ANTONYSAMY, M. A., FANSLOW, W. C., FU, F., LI, W., QIAN, S., TROUTT, A. B. & THOMSON, A. W. 1999. Evidence for a role of IL-17 in organ allograft rejection: IL-17 promotes the functional differentiation of dendritic cell progenitors. J Immunol, 162, 577-84.
35. MOLET, S., HAMID, Q., DAVOINE, F., NUTKU, E., TAHA, R., PAGE, N., OLIVENSTEIN, R., ELIAS, J. & CHAKIR, J. 2001. IL-17 is increased in asthmatic airways and induces human bronchial fibroblasts to produce cytokines. J Allergy Clin Immunol, 108, 430-8.
36. TEUNISSEN, M. B., KOOMEN, C. W., DE WAAL MALEFYT, R., WIERENGA, E. A. & BOS, J. D. 1998. Interleukin-17 and interferon-gamma synergize in the enhancement of proinflammatory cytokine production by human keratinocytes. J Invest Dermatol, 111, 645-9.
37. KURASAWA, K., HIROSE, K., SANO, H., ENDO, H., SHINKAI, H., NAWATA, Y., TAKABAYASHI, K. & IWAMOTO, I. 2000. Increased interleukin-17 production in patients with systemic sclerosis. Arthritis Rheum, 43, 2455-63.
38. OPPMANN, B., LESLEY, R., BLOM, B., TIMANS, J. C., XU, Y., HUNTE, B., VEGA, F., YU, N., WANG, J., SINGH, K., ZONIN, F., VAISBERG, E., CHURAKOVA, T., LIU, M., GORMAN, D., WAGNER, J., ZURAWSKI, S., LIU, Y., ABRAMS, J. S., MOORE, K. W., RENNICK, D., DE WAAL-MALEFYT, R., HANNUM, C., BAZAN, J. F. & KASTELEIN, R. A. 2000. Novel p19 protein engages IL-12p40 to form a cytokine, IL-23, with biological activities similar as well as distinct from IL-12. Immunity, 13, 715-25.
39. WIEKOWSKI, M. T., LEACH, M. W., EVANS, E. W., SULLIVAN, L., CHEN, S. C., VASSILEVA, G., BAZAN, J. F., GORMAN, D. M., KASTELEIN, R. A., NARULA, S. & LIRA, S. A. 2001. Ubiquitous transgenic expression of the IL-23 subunit p19 induces multiorgan inflammation, runting, infertility, and premature death. J Immunol, 166, 7563-70.
40. FRUCHT, D. M. 2002. IL-23: a cytokine that acts on memory T cells. Sci STKE, 2002, pe1.
41. ELPHICK, M., VON HOLLEN, D., PRITCHARD, J. N., NIKANDER, K., HARDAKER, L. E. & HATLEY, R. H. 2015. Factors to consider when selecting a nebulizer for a new inhaled drug product development program. Expert Opin Drug Deliv, 12, 1375-87.
42. YUM, H. K., PARK, I. N., SHIN, B. M. & CHOI, S. J. 2014. Recurrent Pseudomonas aeruginosa Infection in Chronic Lung Diseases: Relapse or Reinfection? Tuberc Respir Dis (Seoul), 77, 172-7.
43. ARULMOZHI, D. K., VEERANJANEYULU, A. & BODHANKAR, S. L. 2005. Migraine: current concepts and emerging therapies. Vascul Pharmacol, 43, 176-87.
44. LASSEN, L. H., HADERSLEV, P. A., JACOBSEN, V. B., IVERSEN, H. K., SPERLING, B. & OLESEN, J. 2002. CGRP may play a causative role in migraine. Cephalalgia, 22, 54-61.
45. HAASS, C. & SELKOE, D. J. 2007. Soluble protein oligomers in neurodegeneration: lessons from the Alzheimer's amyloid beta-peptide. Nat Rev Mol Cell Biol, 8, 101-12.
46. ZELLER, J., POULSEN, K. T., SUTTON, J. E., ABDICHE, Y. N., COLLIER, S., CHOPRA, R., GARCIA, C. A., PONS, J., ROSENTHAL, A. & SHELTON, D. L. 2008. CGRP function-blocking antibodies inhibit neurogenic vasodilatation without affecting heart rate or arterial blood pressure in the rat. Br J Pharmacol, 155, 1093-103.

What is claimed is:

1. A method of administering a lipocalin mutein to a subject, wherein the method comprises administering the lipocalin mutein by inhalation and wherein the administration provides for local exposure to the lipocalin mutein in the respiratory tract.

2. A lipocalin mutein for use in therapy of a subject, wherein the use comprises administering the lipocalin mutein by inhalation, wherein the administration provides for local exposure to the lipocalin mutein in the respiratory tract.

3. The method of, or lipocalin mutein for the use of, claim 1 or 2, wherein about 0.15% or less of the delivered dose of the lipocalin mutein enters the circulatory system.

4. The method of, or lipocalin mutein for the use of, any one of the preceding claims, wherein no systemic exposure of the lipocalin mutein is detectable.

5. The method of, or lipocalin mutein for the use of, claim 4, wherein systemic exposure is defined by absorption into blood.

6. The method of, or lipocalin mutein for the use of, any one of the preceding claims, wherein the delivered dose of the lipocalin mutein is about 0.1 mg to about 1000 mg per administration or about 0.05 μg to about 15 mg per kg body weight per administration.

7. The method of, or lipocalin mutein for the use of, any one of the preceding claims, wherein the delivered dose of the lipocalin mutein is about 0.1 mg to about 5 mg per administration or about 0.05 μg to about 50 μg per kg body weight per administration.

8. The method of, or lipocalin mutein for the use of, any one of the preceding claims, wherein the method or use is for the treatment of a disease of the respiratory tract.

9. The method of, or lipocalin mutein for the use of, any one of the preceding claims, wherein the disease of the respiratory tract is allergic inflammation, allergic asthma, rhinitis, conjunctivitis, lung fibrosis, cystic fibrosis, chronic obstructive pulmonary disease, pulmonary alveolar proteinosis, adult respiratory distress syndrome, or bacterial infections.

10. A method of administering a lipocalin mutein to a subject, wherein the method comprises administering the lipocalin mutein by inhalation and wherein the administration provides for systemic exposure of the lipocalin mutein.

11. A lipocalin mutein for use in therapy of a subject, wherein the use comprises administering the lipocalin mutein by inhalation, wherein the administration provides for systemic exposure of the lipocalin mutein.

12. The method of, or lipocalin mutein for the use of, claim 10 or 11, wherein about 0.3% or more, about 0.4% or more, about 0.5% or more, about 0.6% or more, about 0.7% or more, about 0.8% or more, about 0.9% or more, about 1% or more, about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, or about 15% or more of the delivered dose of the lipocalin mutein enters the circulatory system.

13. The method of, or lipocalin mutein for the use of, any one of claims 10 to 12, wherein the maximum concentration of the lipocalin mutein in blood plasma is achieved at about 0.5 hours to about 10 hours after administration, preferably about 0.5 hours to about 4 hours after administration.

14. The method of, or lipocalin mutein for the use of, any one of claims 10 to 13, wherein the maximum concentration of the lipocalin mutein in blood plasma is about 1 ng per mL or more, preferably from about 1 ng per mL to about 2,000 ng per mL.

15. The method of, or lipocalin mutein for the use of, any one of claims 10 to 14, wherein the lipocalin mutein is administered in a delivered dose of about 0.05 mg to about 1000 mg per administration or about 0.1 μg to about 15 mg per kg body weight per administration.

16. The method of, or lipocalin mutein for the use of, any one of claims 10 to 15, wherein the lipocalin mutein is administered in a delivered dose of about 5 mg to about 1000 mg per administration or about 0.1 mg to about 15 mg per kg body weight per administration.

17. The method of, or lipocalin mutein for the use of, any one of claims 10 to 16, wherein the area under the curve of the serum concentration over the time (AUC_{inf}) of said lipocalin mutein is about 10 h*ng/mL or more, such as from about 10 h*ng/mL to about 16,000 h*ng/m L.

18. The method of, or lipocalin mutein for the use of, any one of claims 10 to 17, wherein the lipocalin mutein has a serum half-life (t_{1/2}) from about 2 hours to about 10 hours.

19. The method of, or lipocalin mutein for the use of, any one of claims 10 to 18, wherein the systemic exposure to the lipocalin mutein provides an improved therapeutic effect compared to systemically administering the same or a comparable bioavailable amount of a lipocalin mutein.

20. The method of, or lipocalin mutein for the use of, any one of claims 10 to 19, wherein the method or use is for the treatment of a disease of the respiratory tract.

21. The method of, or lipocalin mutein for the use of, any one of claims 10 to 20, wherein the disease of the respiratory tract is allergic inflammation, allergic asthma, rhinitis, conjunctivitis, lung fibrosis, cystic fibrosis, chronic obstructive pulmonary disease, pulmonary alveolar proteinosis, adult respiratory distress syndrome, or bacterial infections.

22. The method of, or lipocalin mutein for the use of, any one of claims 10 to 19, wherein the method or use is for the treatment of a systemic disease.

23. The method of, or lipocalin mutein for the use of, any one of claims 10 to 19, wherein the method or use is for the treatment of a disease that affects an organ or tissue other than a disease of the respiratory tract.

24. The method of, or lipocalin mutein for the use of, any one claims 10 to 19, 22, and 23, wherein the method or use is for the treatment of cancer, anemia, a pain disorder, an inflammatory disease, a cardiovascular disease, a neurodegenerative disease, an allergic disease, such as rhinitis, conjunctivitis, dermatitis, or food allergies, or a bacterial infection, such as a Pseudomonas aeruginosa infection.

25. The method of, or lipocalin mutein for the use of, any one of claims 10 to 24, wherein the onset of the effect of the lipocalin mutein is faster than the onset when the lipocalin mutein is administered subcutaneously.

26. The method of, or lipocalin mutein for the use of, any one of claims 10 to 24, wherein the onset of the effect of the lipocalin mutein is about as fast or faster than the onset when the lipocalin mutein is administered systemically.

27. The method of, or lipocalin mutein for the use of, any one of claims 10 to 24, wherein the onset of the effect of the lipocalin mutein in combination with FDKP is about as fast or faster than the onset when the lipocalin mutein is administered by inhalation alone.

28. The method of, or lipocalin mutein for the use of, any one of the preceding claims, wherein administration is once, once a week, twice a week, three times a week, four times a week, five times a week, six times a week, once a day, twice a day.

29. The method of, or lipocalin mutein for the use of, any of the preceding claims, wherein the lipocalin mutein is administered in nebulized form.

30. The method of, or lipocalin mutein for the use of, any of the preceding claims, wherein the lipocalin mutein is administered as dry powder.

31. The method of, or lipocalin mutein for the use of, any of the preceding claims, wherein the lipocalin mutein is administered as a liquid spray.

32. The method of, or lipocalin mutein for the use of, any of the preceding claims, wherein the lipocalin mutein is administered to a human subject.

33. The method of, or composition for the use of, any one of the preceding claims, wherein the lipocalin mutein is a mutein of human tear lipocalin (hTlc) or human neutrophil gelatinase-associated lipocalin (hNGAL).
